SEQUENCE LISTING (42 sequences)

SEQ ID NO: 1 - DNA, 20 bp, Artificial Sequence
  Description of Artificial Sequence Synthetic oligonucleotide
aaggcagtggtttcttacga

SEQ ID NO: 2 - DNA, 20 bp, Artificial Sequence
  Description of Artificial Sequence Synthetic oligonucleotide
ggagtggaaacgcttcatcg

SEQ ID NO: 3 - DNA, 20 bp, Artificial Sequence
  Description of Artificial Sequence Synthetic oligonucleotide
agagatcaaacagctcaccg

SEQ ID NO: 4 - DNA, 20 bp, Artificial Sequence
  Description of Artificial Sequence Synthetic oligonucleotide
aggactcatctgatgatgtg

SEQ ID NO: 5 - DNA, 20 bp, Artificial Sequence
  Description of Artificial Sequence Synthetic oligonucleotide
aagaagttacatcttaacac

SEQ ID NO: 6 - DNA, 20 bp, Artificial Sequence
  Description of Artificial Sequence Synthetic oligonucleotide
cactctatgaggatagtcat

SEQ ID NO: 7 - DNA, 20 bp, Artificial Sequence
  Description of Artificial Sequence Synthetic oligonucleotide
tgatgcgccggtaatcggag

SEQ ID NO: 8 - DNA, 20 bp, Artificial Sequence
  Description of Artificial Sequence Synthetic oligonucleotide
tggtgcgcgacagtcgccaa

SEQ ID NO: 9 - DNA, 20 bp, Artificial Sequence
  Description of Artificial Sequence Synthetic oligonucleotide
gtaccaatgagtgcaaagcg

SEQ ID NO: 10 - DNA, 20 bp, Artificial Sequence
  Description of Artificial Sequence Synthetic oligonucleotide
atgacacttacggtactcgt

SEQ ID NO: 11 - DNA, 55 bp, Artificial Sequence
  Description of Artificial Sequence Synthetic primer
tcgtcggcagcgtcagatgtgtataagagacagttgtggaaaggacgaaacaccg

SEQ ID NO: 12 - DNA, 57 bp, Artificial Sequence
  Description of Artificial Sequence Synthetic primer
gtctcgtgggctcggagatgtgtataagagacagccactttttcaagttgataacgg

SEQ ID NO: 13 - DNA, 22 bp, Artificial Sequence
  Description of Artificial Sequence Synthetic primer
gacaaggagatgcaggtattgg

SEQ ID NO: 14 - DNA, 22 bp, Artificial Sequence
  Description of Artificial Sequence Synthetic primer
tcccgtagagatccacaaaagt

SEQ ID NO: 15 - DNA, 21 bp, Artificial Sequence
  Description of Artificial Sequence Synthetic primer
acagcaaggcgaaaaaggatg

SEQ ID NO: 16 - DNA, 19 bp, Artificial Sequence
  Description of Artificial Sequence Synthetic primer
tggtggaccactcggatga

SEQ ID NO: 17 - DNA, 20 bp, Artificial Sequence
  Description of Artificial Sequence Synthetic primer
tccgacagtggttgatcgac

SEQ ID NO: 18 - DNA, 22 bp, Artificial Sequence
  Description of Artificial Sequence Synthetic primer
cctcacgattgtagtcctgctt

SEQ ID NO: 19 - DNA, 19 bp, Artificial Sequence
  Description of Artificial Sequence Synthetic primer
tgctgcccactgtaacgtg

SEQ ID NO: 20 - DNA, 22 bp, Artificial Sequence
  Description of Artificial Sequence Synthetic primer
ggtaggtgaaggatagccacat

SEQ ID NO: 21 - DNA, 20 bp, Artificial Sequence
  Description of Artificial Sequence Synthetic primer
ccactctcgaccctacatgg

SEQ ID NO: 22 - DNA, 22 bp, Artificial Sequence
  Description of Artificial Sequence Synthetic primer
ggcccccaaagtgacatttatt

SEQ ID NO: 23 - DNA, 20 bp, Artificial Sequence
  Description of Artificial Sequence Synthetic primer
cgccaccagttcgccatgga

SEQ ID NO: 24 - DNA, 20 bp, Artificial Sequence
  Description of Artificial Sequence Synthetic primer
tacagcccggggagcatcgt

SEQ ID NO: 25 - protein, 125 aa, Homo sapiens
MPHSSDSSDSSFSRSPPPGKQDSSDDVRRVQRREKNRIAAQKSRQRQTQKADTLHLESEDLEKQNAALRKEIKQLTEELKYFTSVLNSHEPLCSVLAASTPSPPEVVYSAHAFHQPHVSSPRFQP

SEQ ID NO: 26 - protein, 125 aa, Mus musculus
MPHSSDSSDSSFSRSPPPGKQDSSDDVRKVQRREKNRIAAQKSRQRQTQKADTLHLESEDLEKQNAALRKEIKQLTEELKYFTSVLSSHEPLCSVLASGTPSPPEVVYSAHAFHQPHISSPRFQP

SEQ ID NO: 27 - DNA, 913 bp, Homo sapiens
aaagcgagcgacatgtccctttggggagcagtccctctgcaccccagagtgaggaggacgcaggggtcagaggtggctacagggcaggcagaggaggcacctgtagggggtggtgggctggtggcccaggagaagtcaggaagggagcccagctggtgacaagagagcccagaggtgcctggggctgagtgtgagagcccggaagatttcagccatgcctcacagctccgacagcagtgactccagcttcagccgctctcctccccctggcaaacaggactcatctgatgatgtgagaagagttcagaggagggagaaaaatcgtattgccgcccagaagagccgacagaggcagacacagaaggccgacaccctgcacctggagagcgaagacctggagaaacagaacgcggctctacgcaaggagatcaagcagctcacagaggaactgaagtacttcacgtcggtgctgaacagccacgagcccctgtgctcggtgctggccgccagcacgccctcgccccccgaggtggtgtacagcgcccacgcattccaccaacctcatgtcagctccccgcgcttccagccctgagcttccgatgcggggagagcagagcctcgggaggggcacacagactgtggcagagctgcgcccatcccgcagaggcccctgtccacctggagacccggagacagaggcctggacaaggagtgaacacgggaactgtcacgactggaagggcgtgaggcctcccagcagtgccgcagcgtttcgaggggcgtgtgctggaccccaccactgtgggttgcaggcccaatgcagaagagtattaagaaagatgctcaagtcccatggcacagagcaaggcgggcagggaacggttatttttctaaataaatgctttaaaagaaa

SEQ ID NO: 28 - DNA, 904 bp, Mus musculus
gcagtccctctgcacccgagagagaggaggacgcaggggtctgtcagaggttgctgttgggcaagcaggggaggtacctgtggaaggtggtgtgctggtggccccctagcagtcaagaaggggagccagctagtgagaagatcgcccagaggcatctgggacggtgtgggagagcccggaagattagaaccatgcctcacagctccgacagcagtgactccagcttcagccgctctcctccccctggcaaacaggactcatctgatgatgtgaggaaagttcagaggagagagaagaatcgcatcgctgcccagaagagccgacagagacagacacagaaagccgacacccttcacctggagagtgaggacctggagaaacagaacgcagctctccgcaaagagatcaaacagctcaccgaggagctcaagtacttcacatcagtgctgagcagccacgagcccctgtgctccgtgctggccagtggcaccccctcgccccccgaggtggtatacagtgcccatgccttccaccagcctcacatcagctcgccacgcttccagccctgaccttctggacaagaagggcgatgctactcccgtgatcccttggaggggcatgtaaactgaggccgggctgccctcatacctctacccagaggcccagtggcagaggcctggacaagtattgaacacaagaactgtagtggtcagagggacttaaggcctcccagggaagtatagtcaatgtactggactctcccagggaagtcgagccaatgtactggacccaaaaaatgacaagtcaaccctggactgtcatgaatgatgcccaaaatacacagcacagagggaggagggcagggggtggatagttttctaaataaatattttctaaaaaacca

SEQ ID NO: 29 - DNA, 23 bp, Artificial Sequence
  Description of Artificial Sequence Synthetic oligonucleotide
  modified_base (21)..(21): a, c, t, or g
ttcacaccatcacgacgcgtngg

SEQ ID NO: 30 - DNA, 23 bp, Artificial Sequence
  Description of Artificial Sequence Synthetic oligonucleotide
  modified_base (21)..(21): a, c, t, or g
tgggggcagcttggccgctcngg

SEQ ID NO: 31 - DNA, 23 bp, Artificial Sequence
  Description of Artificial Sequence Synthetic oligonucleotide
  modified_base (21)..(21): a, c, t, or g
tatgccccctgatgacccacngg

SEQ ID NO: 32 - DNA, 23 bp, Artificial Sequence
  Description of Artificial Sequence Synthetic oligonucleotide
  modified_base (21)..(21): a, c, t, or g
aaggaggtcttctcctgccgngg

SEQ ID NO: 33 - DNA, 23 bp, Artificial Sequence
  Description of Artificial Sequence Synthetic oligonucleotide
  modified_base (21)..(21): a, c, t, or g
gtgatgggcacgtcgggccgngg

SEQ ID NO: 34 - DNA, 23 bp, Artificial Sequence
  Description of Artificial Sequence Synthetic oligonucleotide
  modified_base (21)..(21): a, c, t, or g
cagctccctctagtcccgcgngg

SEQ ID NO: 35 - RNA, 23 nt, Artificial Sequence
  Description of Artificial Sequence Synthetic oligonucleotide
  modified_base (21)..(21): a, c, u, or g
gcuuguggauguugcggaagngg

SEQ ID NO: 36 - DNA, 20 bp, Artificial Sequence
  Description of Artificial Sequence Synthetic oligonucleotide
ttcacaccatcacgacgcgt

SEQ ID NO: 37 - DNA, 20 bp, Artificial Sequence
  Description of Artificial Sequence Synthetic oligonucleotide
tgggggcagcttggccgctc

SEQ ID NO: 38 - DNA, 20 bp, Artificial Sequence
  Description of Artificial Sequence Synthetic oligonucleotide
tatgccccctgatgacccac

SEQ ID NO: 39 - DNA, 20 bp, Artificial Sequence
  Description of Artificial Sequence Synthetic oligonucleotide
aaggaggtcttctcctgccg

SEQ ID NO: 40 - DNA, 20 bp, Artificial Sequence
  Description of Artificial Sequence Synthetic oligonucleotide
gtgatgggcacgtcgggccg

SEQ ID NO: 41 - DNA, 20 bp, Artificial Sequence
  Description of Artificial Sequence Synthetic oligonucleotide
cagctccctctagtcccgcg

SEQ ID NO: 42 - DNA, 20 bp, Artificial Sequence
  Description of Artificial Sequence Synthetic oligonucleotide
ggtagagggttactacccgg

(19) United States
(12) Patent Application Publication
(10) Pub. No.: US 2022/0226380 A1
(43) Pub. Date: Jul. 21, 2022

(54) GENE KNOCK-OUTS TO IMPROVE T CELL FUNCTION

(71) Applicant: ST. JUDE CHILDREN'S RESEARCH HOSPITAL, INC., Memphis, TN (US)

(72) Inventors: Hongbo CHI, Memphis, TN (US); Jun WEI, Memphis, TN (US); Terrence GEIGER, Memphis, TN (US); Wenting ZHENG, Memphis, TN (US)

(73) Assignee: ST. JUDE CHILDREN'S RESEARCH HOSPITAL, INC., Memphis, TN (US)

(21) Appl. No.: 17/605,639

(22) PCT Filed: Apr. 23, 2020
(86) PCT No.: PCT/US2020/029533

§ 371 Date: Oct. 22, 2021

Related U.S. Application Data
(60) Provisional application No. 62/838,060, filed on Apr. 24, 2019.
(60) Provisional application No. 62/912,231, filed on Oct. 8, 2019.

(51) Int. Cl. A61K 35/17; C12N 15/90; C12N 15/11; C12N 9/22; C12N 9/10; C12N 9/16; A61P 35/00; C07K 14/47; A61K 38/46; A61K 31/7088; C12N 15/86; C07K 16/28; C07K 14/705; C07K 14/725

(52) U.S. Cl. CPC A61K 35/17; A61K 48/00; C12N 15/11; C12N 9/22; C12N 9/1085; C12Y 205/01026; C12N 9/16; C12Y 301/03048; A61P 35/00; C07K 14/4702; A61K 38/465; A61K 31/7088; C12N 15/86; C07K 16/2803; C07K 14/70517; C07K 14/70578; C07K 14/7051; C12N 2310/20; C12N 2800/80; C12N 15/907

(57) ABSTRACT

The present application provides methods of enhancing T cell function (e.g., expansion, persistence and/or effector functions), particularly by genetic modification of the Regnase-1, Batf, and additional genes (alone or in combination). The application also provides modified T cells manufactured using the methods provided by this invention and related pharmaceutical compositions. The application further provides methods of using the modified T cells for treating a disease (e.g., a cancer or an infectious disease).

### CROSS-REFERENCE TO RELATED APPLICATIONS

This application claims priority to U.S. Provisional Application Nos. 62/838,060, filed Apr. 24, 2019, and 62/912,231, filed Oct. 8, 2019, both of which are herein incorporated by reference in their entirety.

### FIELD

The application relates to methods of enhancing T cell function, particularly by genetic modification of the Regnase-1, Batf, and additional genes (alone or in combination). The application further relates to the modified T cells and related pharmaceutical compositions. The application further relates to the therapeutic use of the modified T cells for treating diseases.

### BACKGROUND

Adoptive cell therapy (ACT) using engineered T cells has produced unprecedented results in the clinic and represents a new paradigm in cancer immunotherapy. However, the therapeutic efficacy, especially in solid tumors, is often limited by poor in vivo expansion, persistence and function of adoptively transferred T cells^{1,2}. Furthermore, T cell fate decisions in the tumor microenvironment (TME) and the underlying processes remain elusive.

CD8⁺ T cells play a pivotal role in the control of cancer. The efficacy of ACT, including the use of T cells engineered to express chimeric antigen receptors (CARs), depends upon T cell longevity and their differentiation state^{1,2}. Paradoxically, fully differentiated effector CD8⁺ T cells have been shown to have reduced antitumor efficacy and exhibit poor in vivo persistence^{2,3}, while the long-term persistence of Tet2-mutated T cells, despite the impaired effector function, is associated with tumor remission in a clinical case⁴. Another major challenge of ACT against solid tumors is that antitumor T cell responses can be blunted in the highly immunosuppressive TME^{1,2,5}, as evidenced by the poor accumulation of adoptively transferred T cells and limited therapeutic efficacy in human solid tumors and mouse ACT models.

As such, there is a need in the art for strategies to enhance T cell function, in particular T cell expansion, persistence and/or effector function, to allow for improved ACT efficacy in cancer immunotherapy⁶. The present invention addresses this and other related needs.

### SUMMARY OF THE INVENTION

In one aspect provided herein is a method of enhancing expansion and/or persistence and/or an anti-tumor or an anti-infection function of a T cell, comprising modifying a Regnase-1 (REGNASE-1, Zc3h12a, MCPIP 1) gene or gene product in the T cell such that the expression and/or function of Regnase-1 in the T cell is reduced or eliminated.

In some embodiments, the T cell is selected from a CD8⁺ αβ T cell receptor (TCR) T cell, a CD4⁺ αβ TCR T cell, a regulatory T cell, a natural killer T (NKT) cell, and a γϵ T cell. In some embodiments, the T cell is a CD8⁺ αβ TCR T cell. In some embodiments, the T cell is a CD4⁺ αβ TCR T cell.

In some embodiments, the T cell is further engineered to express a T cell receptor or a chimeric antigen receptor (CAR). In some embodiments, the CAR targets a tumor antigen or an infectious antigen.

In some embodiments, the modifying step comprises disrupting the Regnase-1 gene with a site-specific nuclease. In some embodiments, the site-specific nuclease comprises a Cas protein and a guide RNA. In some embodiments, the Cas protein is a Cas9 protein. In some embodiments, the guide RNA is a single guide RNA (sgRNA). In some embodiments, the sgRNA targets Regnase-1 . In some embodiments, the sgRNA comprises TTCACACCATCACGACGCGTNGG (SEQ ID NO: 29), CAGCTCCCTCTAGTCCCGCGNGG (SEQ ID NO: 34), TTCACACCATCACGACGCGT (SEQ ID NO: 36) or CAGCTCCCTCTAGTCCCGCG (SEQ ID NO: 41), or a nucleotide sequence having at least 80% identity thereof. In some embodiments, the site-specific nuclease comprises a zinc finger nuclease (ZFN), a TALEN nuclease, or a mega-TALEN nuclease.

In some embodiments, the modifying step comprises silencing a Regnase-1 mRNA with an RNA interference (RNAi) molecule or an antisense oligonucleotide. In some embodiments, the RNAi molecule is a small interfering RNA (siRNA) or a small hairpin RNA (shRNA).

In some embodiments, the modifying step comprises inhibiting a Regnase-1 protein with one or more of a small molecule inhibitor, a peptide, an antibody or antibody fragment, and an aptamer.

In some embodiments, in vivo accumulation of the T cell is improved more than 100-fold as compared an unmodified T cell at day 7 after the Regnase-1 modification.

In some embodiments, the method further comprises modifying one or more additional genes or gene products alone or in combination with Regnase-1 in the T cell such that the expression and/or function of the additional gene(s) or gene product(s) in the T cell is reduced or eliminated, wherein the additional gene(s) or gene product(s) are selected from Ptpn2, Socs 1 , Agps, Rc3h1, and Rcor 1 .

In some embodiments, modifying of one or more additional genes comprises disrupting the gene(s) with a site-specific nuclease. In some embodiments, the site-specific nuclease comprises a Cas protein and a guide RNA. In some embodiments, the Cas protein is a Cas9 protein. In some embodiments, the guide RNA is a single guide RNA (sgRNA). In some embodiments, the site-specific nuclease comprises a zinc finger nuclease (ZFN), a TALEN nuclease, or a mega-TALEN nuclease.

In some embodiments, modifying of one or more additional gene products comprises administering an RNA interference (RNAi) molecule or an antisense oligonucleotide. In some embodiments, the RNAi molecule is a small interfering RNA (siRNA) or a small hairpin RNA (shRNA).

In some embodiments, modifying of one or more additional gene products comprises administering one or more of a small molecule inhibitor, a peptide, an antibody or antibody fragment, and an aptamer.

In another aspect provided herein is a modified T cell produced by any of the methods described above. In some embodiments, the T cell is a CD8⁺ T cell. In some embodiments, the T cell is derived from a blood, marrow, tissue, or tumor sample. In some embodiments, the T cell is an allogeneic T cell. In some embodiments, the T cell is an autologous T cell. In some embodiments, the T cell has been activated and/or expanded ex vivo.

In another aspect provided herein is a pharmaceutical composition comprising the modified T cell described above and a pharmaceutically acceptable carrier and/or excipient.

In another aspect provided herein is a method of treating a disease in a subject in need thereof comprising administering to the subject an effective amount of the modified T cells or the pharmaceutical composition described above. In some embodiments, the modified T cells are autologous cells. In some embodiments, the modified T cells are allogeneic cells. In some embodiments, the disease is a cancer or an infectious disease. In some embodiments, the cancer is a solid tumor. In some embodiments, the cancer is melanoma, colon cancer, breast cancer, or brain cancer. In some embodiments, the cancer is a blood cancer. In some embodiments, the cancer is a lymphoma, leukemia, or multiple myeloma.

In some embodiments, the method comprises: a) isolating a T cell from the subject or a donor; b) modifying a Regnase-1 gene or gene product in the T cell such that the expression and/or function of Regnase-1 in the T cell is reduced or eliminated; c) optionally, activating and/or expanding the T cell before or after step b); and d) administering an effective amount of the modified T cells to the subject.

In various embodiments, the subject is a human.

In another aspect provided herein is a method of enhancing expansion and/or persistence and/or an anti-tumor or an anti-infection function of a T cell, comprising increasing the expression of Batf gene and/or enhancing the function of BATF protein in the T cell.

In some embodiments, the T cell is selected from a CD8⁺ αβ T cell receptor (TCR) T cell, a CD4⁺ αβ TCR T cell, a regulatory T cell, a natural killer T (NKT) cell, and a γϵ T cell. In some embodiments, the T cell is a CD8⁺ αβ TCR T cell. In some embodiments, the T cell is a CD4⁺ αβ TCR T cell.

In some embodiments, the T cell is further engineered to express a T cell receptor or a chimeric antigen receptor (CAR). In some embodiments, the CAR targets a tumor antigen or an infectious antigen.

In some embodiments, the method comprises introducing into the T cell a polynucleotide encoding a BATF protein, or functional fragment or derivative thereof.

In some embodiments, the polynucleotide encoding a BATF protein comprises the nucleotide sequence of SEQ ID NO: 27, or a nucleotide sequence having at least 80% identity therof. In some embodiments, the BATF protein encoded by the polynucleotide comprises the amino acid sequence of SEQ ID NO: 25, or an amino acid sequence having at least 80% identity therof.

In some embodiments, the polynucleotide encoding a BATF protein, or functional fragment or derivative thereof, is introduced into the T cell in a recombinant vector. In some embodiments, the recombinant vector is a viral vector. In some embodiments, the viral vector is a retroviral vector, a lentiviral vector, an adenoviral vector, an adeno-associated virus vector, an alphaviral vector, a herpes virus vector, or a vaccinia virus vector. In some embodiments, the viral vector is a retroviral vector. In some embodiments, the recombinant vector is a non-viral RNA and/or DNA vector.

In some embodiments, the method further comprises modifying one or more additional genes or gene products, alone or in combination, in the T cell such that the expression and/or function of the additional gene(s) or gene product(s) in the T cell is reduced or eliminated, wherein the additional gene(s) or gene product(s) are selected from Regnase-1 (REGNASE-1, Zc3h12a, MCPIP1), Ptpn2, Socs1, Agps, Rc3h1, and Rcor1. In some embodiments, the additional gene(s) or gene product(s) is Regnase-1 (REGNASE-1, Zc3h12a, MCPIP1).

In some embodiments, modifying of one or more additional genes comprises disrupting the gene(s) with a site-specific nuclease. In some embodiments, the site-specific nuclease comprises a Cas protein and a guide RNA. In some embodiments, the Cas protein is a Cas9 protein. In some embodiments, the guide RNA is a single guide RNA (sgRNA). In some embodiments, the sgRNA comprises TTCACACCATCACGACGCGTNGG (SEQ ID NO: 29), CAGCTCCCTCTAGTCCCGCGNGG (SEQ ID NO: 34), TTCACACCATCACGACGCGT (SEQ ID NO: 36) or CAGCTCCCTCTAGTCCCGCG (SEQ ID NO: 41), or a nucleotide sequence having at least 80% identity thereof In some embodiments, the site-specific nuclease comprises a zinc finger nuclease (ZFN), a TALEN nuclease, or a mega-TALEN nuclease.

In some embodiments, modifying of one or more additional gene products comprises administering an RNA interference (RNAi) molecule or an antisense oligonucleotide. In some embodiments, the RNAi molecule is a small interfering RNA (siRNA) or a small hairpin RNA (shRNA).

In some embodiments, modifying of one or more additional gene products comprises administering one or more of a small molecule inhibitor, a peptide, an antibody or antibody fragment, and an aptamer.

In another aspect provided herein is a method of enhancing expansion and/or persistence and/or an anti-tumor or an anti-infection function of a T cell, comprising modifying a Regnase-1 (REGNASE-1, Zc3h12a, MCPIP 1) gene or gene product in the T cell such that the expression and/or function of Regnase-1 in the T cell is reduced or eliminated and increasing the expression of Batf gene and/or enhancing the function of BATF protein in the T cell. In some embodiments, the method further comprises modifying one or more additional genes or gene products in the T cell such that the expression and/or function of the additional gene(s) or gene product(s) in the T cell is reduced or eliminated, wherein the additional gene(s) or gene product(s) are selected from Ptpn2, Socs 1 , Agps, Rc3h1, and Rcor 1 .

In various embodiments, the site-specific nuclease, RNAi molecule, antisense oligonucleotide, peptide, small molecule inhibitor, antibody or antibody fragment, or aptamer is introduced into the T cell via a physical means. In some embodiments, the physical means is electroporation, microinjection, magnetofection, ultrasound, a ballistic or hydrodynamic method, or a combination thereof In some embodiments, the physical means is electroporation.

In various embodiments, the site-specific nuclease, RNAi molecule, antisense oligonucleotide, peptide, antibody or antibody fragment, or aptamer is introduced into the T cell in a recombinant vector. In some embodiments, the recombinant vector is a viral vector. In some embodiments, the viral vector is a retroviral vector, a lentiviral vector, an adenoviral vector, an adeno-associated virus vector, an alphaviral vector, a herpes virus vector, or a vaccinia virus vector. In some embodiments, the recombinant vector is a non-viral RNA and/or DNA vector.

In another aspect provided herein is a modified T cell produced by any of the method described above that involves increasing the expression of Batf gene and/or enhancing the function of BATF protein in the T cell. In some embodiments, the T cell is a CD8⁺ T cell. In some embodiments, the T cell is derived from a blood, marrow, tissue, or tumor sample. In some embodiments, the T cell is an allogeneic T cell. In some embodiments, the T cell is an autologous T cell. In some embodiments, the T cell has been activated and/or expanded ex vivo.

In another aspect provided herein is a pharmaceutical composition comprising the modified T cell described above and a pharmaceutically acceptable carrier and/or excipient.

In another aspect provided herein is a method of treating a disease in a subject in need thereof comprising administering to the subject an effective amount of the modified T cells described above or the pharmaceutical composition described above. In some embodiments, the modified T cells are autologous cells. In some embodiments, the modified T cells are allogeneic cells. In some embodiments, the disease is a cancer or an infectious disease. In some embodiments, the cancer is a solid tumor. In some embodiments, the cancer is melanoma, colon cancer, breast cancer, or brain cancer. In some embodiments, the cancer is a blood cancer. In some embodiments, the cancer is a lymphoma, leukemia, or multiple myeloma.

In some embodiments, the method comprises a) isolating a T cell from the subject or a donor; b) increasing the expression of Batf gene and/or enhancing the function of BATF protein in the T cell; c) optionally, activating and/or expanding the T cell before or after step b); and d) administering an effective amount of the modified T cells to the subject.

In various embodiments of the methods described above, the subject is a human.

In another aspect provided herein is a method of improving mitochondrial biogenesis and/or function in a T cell comprising modifying a Regnase-1 (REGNASE-1, Zc3h12a, MCPIP 1) gene or gene product in the T cell such that the expression and/or function of Regnase-1 in the T cell is reduced or eliminated and/or increasing the expression of Batf gene and/or enhancing the function of BATF protein in the T cell. In some embodiments, the method further comprises modifying one or more additional genes or gene products alone or together with Regnase-1 in the T cell such that the expression and/or function of the additional gene(s) or gene product(s) in the T cell is reduced or eliminated, wherein the additional gene(s) or gene product(s) are selected from Ptpn2, Socs1, Agps, Rc3h1, and Rcor 1 .

In another aspect provided herein is an isolated polynucleotide, comprising the nucleotide sequence of any one of SEQ ID NOs: 1-9, 29-34 and 36-42, ora nucleotide sequence having at least 80% identity thereof In some embodiments, the isolated polynucleotide comprises the nucleotide sequence of SEQ ID NO: 1 or 2. In some embodiments, the isolated polynucleotide comprises the nucleotide sequence of SEQ ID NO: 29, 34, 36 or 41. In some embodiments, the polynucleotide is a guide RNA. In some embodiments, the guide RNA is a single guide RNA (sgRNA).

In another aspect provided herein is a method of modifying a gene in a cell, comprising introducing into the cell a site-specific nuclease. In some embodiments, the cell is a T cell. In some embodiments, the T cell is selected from a CD8⁺ αβ T cell receptor (TCR) T cell, a CD4⁺ αβ TCR T cell, a regulatory T cell, a natural killer T (NKT) cell, and a γϵ T cell. In some embodiments, the T cell is a CD8⁺ αβ TCR T cell. In some embodiments, the T cell is a CD4⁺ αβ TCR T cell. In some embodiments, the T cell is further engineered to express a T cell receptor or a chimeric antigen receptor (CAR). In some embodiments, the cell is a human cell.

In some embodiments of the method of modifying a gene in a cell, the site-specific nuclease comprises a Cas protein and one or more guide RNAs. In some embodiments, the Cas protein is a Cas9 protein. In some embodiments, the one or more guide RNAs are one or more single guide RNAs (sgRNAs). In some embodiments, at least one sgRNA targets Regnase-1 . In some embodiments, the sgRNA comprises TTCACACCATCACGACGCGTNGG (SEQ ID NO: 29), CAGCTCCCTCTAGTCCCGCGNGG (SEQ ID NO: 34), TTCACACCATCACGACGCGT (SEQ ID NO: 36) or CAGCTCCCTCTAGTCCCGCG (SEQ ID NO: 41), or a nucleotide sequence having at least 80% identity thereof In some embodiments, the Cas protein and the guide RNA are mixed to form a ribonucleoprotein (RNP) complex.

In some embodiments of the method of modifying a gene in a cell, the site-specific nuclease is introduced into the cell via a physical means. In some embodiments, the physical means is electroporation, microinjection, magnetofection, ultrasound, a ballistic or hydrodynamic method, or a combination thereof. In some embodiments, the physical means is electroporation.

In another aspect provided herein is a method of enhancing expansion and/or persistence and/or an anti-tumor or an anti-infection function of a T cell, comprising modifying one or more genes or gene products thereof in the T cell such that the expression and/or function of gene or gene product in the T cell is reduced or eliminated, wherein the one or more genes are selected from Ptpn2, Socs 1 , Agps, Rc3h1 (Roquin-1) and Rcor1 .

In some embodiments, the T cell is selected from a CD8⁺ αβ T cell receptor (TCR) T cell, a CD4⁺ αβ TCR T cell, a regulatory T cell, a natural killer T (NKT) cell, and a γϵ T cell. In some embodiments, the T cell is a CD8⁺ αβ TCR T cell. In some embodiments, the T cell is a CD4⁺ αβ TCR T cell.

In some embodiments, the T cell is further engineered to express a T cell receptor or a chimeric antigen receptor (CAR). In some embodiments, the CAR targets a tumor antigen or an infectious antigen.

In some embodiments, the modifying step comprises disrupting said one or more genes with a site-specific nuclease. In some embodiments, the site-specific nuclease comprises a Cas protein and a guide RNA. In some embodiments, the Cas protein is a Cas9 protein. In some embodiments, the guide RNA is a single guide RNA (sgRNA). In some embodiments, the sgRNA targets said one or more genes. In some embodiments, the site-specific nuclease comprises a zinc finger nuclease (ZFN), a TALEN nuclease, or a mega-TALEN nuclease.

In some embodiments, the modifying step comprises silencing an mRNA produced from said one or more genes with an RNA interference (RNAi) molecule or an antisense oligonucleotide. In some embodiments, the RNAi molecule is a small interfering RNA (siRNA) or a small hairpin RNA (shRNA).

In some embodiments, the modifying step comprises inhibiting a protein produced from said one or more genes with one or more of a small molecule inhibitor, a peptide, an antibody or antibody fragment, and an aptamer.

In some embodiments, the site-specific nuclease, RNAi molecule, antisense oligonucleotide, peptide, small molecule inhibitor, antibody or antibody fragment, or aptamer is introduced into the T cell via a physical means. In some embodiments, the physical means is electroporation, microinjection, magnetofection, ultrasound, a ballistic or hydrodynamic method, or a combination thereof In some embodiments, the physical means is electroporation.

In various embodiments, the site-specific nuclease, RNAi molecule, antisense oligonucleotide, peptide, antibody or antibody fragment, or aptamer is introduced into the T cell in a recombinant vector. In some embodiments, the recombinant vector is a viral vector. In some embodiments, the viral vector is a retroviral vector, a lentiviral vector, an adenoviral vector, an adeno-associated virus vector, an alphaviral vector, a herpes virus vector, or a vaccinia virus vector. In some embodiments, the recombinant vector is a non-viral RNA and/or DNA vector.

In another aspect, provided herein is a modified T cell produced by the method described above that involves modifying one or more genes selected from one or more genes are selected from Ptpn2, Socs1 , Agps, Rc3h1 (Roquin-1) and Rcor 1 , or gene products thereof. In some embodiments, the T cell is a CD8⁺ T cell. In some embodiments, the T cell is derived from a blood, marrow, tissue, or tumor sample. In some embodiments, the T cell is an allogeneic T cell. In some embodiments, the T cell is an autologous T cell. In some embodiments, the T cell has been activated and/or expanded ex vivo.

In another aspect, provided herein is a pharmaceutical composition comprising the modified T cell described above and a pharmaceutically acceptable carrier and/or excipient.

In another aspect, provided herein is a method of treating a disease in a subject in need thereof comprising administering to the subject an effective amount of the modified T cells described above or the pharmaceutical composition described above. In some embodiments, the modified T cells are autologous cells. In some embodiments, the modified T cells are allogeneic cells. In some embodiments, the disease is a cancer or an infectious disease. In some embodiments, the cancer is a solid tumor. In some embodiments, the cancer is melanoma, colon cancer, breast cancer, or brain cancer. In some embodiments, the cancer is a blood cancer. In some embodiments, the cancer is a lymphoma, leukemia, or multiple myeloma.

In some embodiments, the method comprises:
a) isolating a T cell from the subject or a donor;
b) modifying one or more genes or gene products thereof in the T cell such that the expression and/or function of the gene or gene product in the T cell is reduced or eliminated, wheren the one or more genes are selected from Ptpn2, Socs1 , Agps, Rc3h1 (Roquin-1) and Rcor 1;
c) optionally, activating and/or expanding the T cell before or after step b); and
d) administering an effective amount of the modified T cells to the subject.

In various embodiments of the methods described above, the subject is a human.

These and other aspects of the present invention will be apparent to those of ordinary skill in the art in the following description, claims and drawings.

### BRIEF DESCRIPTION OF THE DRAWINGS

U.S. Provisional Application Nos. 62/838,060 and 62/912,231 contain copies of several of the drawing(s) below in color, which can be provided by the United States Patent and Trademark Office upon request and payment of the necessary fee.

FIGS. 1A-1G illustrate the identification of Regnase-1 as a major negative regulator of CD8⁺ T cell antitumor responses using in vivo CRISPR-Cas9 mutagenesis screening. (FIG. 1A) Diagram of the screening system. Naive Cas9-expressing OT-I cells were transduced with lentiviral sgRNA metabolic library and expanded in vitro before adoptive transfer into B16-Ova melanoma-bearing mice. OT-I cells were purified from tumor-infiltrating lymphocytes (TILs) at 7 days after transfer, and library representation in TILs and pre-transfer (input) OT-I cells was examined by deep sequencing of sgRNA cassette. (FIG. 1B) Scatterplot of the enrichment of each gene versus its adjusted P values. Gene enrichment was calculated by averaging the enrichment of their 6 sgRNAs in tumor-infiltrating OT-I cells relative to input (log₂ ratio (TIL/input)), with the most extensively enriched (black solid circle) and selective depleted (stripe-filled circle) genes (adjusted P<0.05), as well as ‘dummy’ genes (empty circle; generated by random combinations of 6 out of 1,000 non-targeting control sgRNAs per ‘dummy’ gene) highlighted. (FIG. 1C) Diagram of in vivo dual transfer system. OT-I cells transduced with sgRNA viral vectors expressing distinct fluorescent proteins were mixed and transferred into the same tumor-bearing hosts where further analyses were performed. (FIG. 1D) Representative images (left) and quantification of relative OT-I cell number per area (μm²) normalized to input (right) in the whole tumor section (n=4 mice). OT-I cells transduced with non-targeting control sgRNA- (referred to as “control sgRNA” hereafter) (mCherry⁺; red) and sgRegnase-1 (Ametrine⁺; green) were mixed at a 10:1 ratio and transferred into tumor-bearing mice, followed by imaging analysis of tumors at day 7. Scale bars, 500 μm. (FIG. 1E) Immunoblot analysis of Regnase-1 expression in control sgRNA- and sgRegnase-1-transduced OT-I cells isolated from TILs at 7 days after adoptive transfer. (FIG. 1F-1G) OT-I cells transduced with control sgRNA-(mCherry⁺) and sgRegnase-1 (Ametrine⁺) were mixed at a 10:1 ratio (to obtain sufficient numbers of control sgRNA-transduced cells at later stages for analysis) and transferred into tumor-bearing mice, followed by analyses of the proportion of donor-derived OT-I cells in total CD8α⁺ cells (FIG. 1F), and quantification of relative OT-I cell percentage in CD8α⁺ cells normalized to input (FIG. 1G, left) and normalized OT-I cell number relative to input (FIG. 1G, right) in the spleen and tumor at days 7, 14 and 21 after transfer (n=10 mice at days 7 and 14, n=6 mice at day 21). Cell number in the tumor is shown as cell number per gram tissue. Numbers in plots indicate frequencies of OT-I cells in gates (FIG. 1F). Numbers above bar graphs indicate fold change of sgRegnase-1- versus control sgRNA-transduced OT-I cells (FIG. 1G). Mean±s.e.m. in FIGS. 1D and 1G. *P<0.05; **P<0.01; ***P<0.001; two-tailed unpaired Student's t-test in FIGS. 1D and 1G. Data are representative of one (FIG. 1B) or two (FIGS. 1D-1F) independent experiments, or pooled from two (FIG. 1G) independent experiments.

FIGS. 2A-2F show the enhanced therapeutic efficacy of Regnase-1-deficient engineered CD8⁺ T cells against solid and blood cancers. (FIGS. 2A-2B) OT-I cells (5×10⁶) transduced with control sgRNA (Ametrine⁺) (n=7 recipients) or sgRegnase-1 (Ametrine⁺) (n=8 recipients) were transferred into mice at day 12 after B16-Ova melanoma engraftment, followed by analyses of tumor size (FIG. 2A) and mouse survival (FIG. 2B). Non-treatment control group mice received no T cell transfer (n=5 recipients). (FIGS. 2C-2D) Pmel-1 cells (5×10⁶) transduced with control sgRNA (Ametrine⁺) (n=5 recipients) or sgRegnase-1 (Ametrine⁺) (n=5 recipients) were transferred into mice at day 12 after B16-F10 melanoma engraftment, followed by analyses of tumor size (FIG. 2C) and mouse survival (FIG. 2D). Non-treatment control group mice received no T cell transfer (n=5 recipients). (FIGS. 2E-2F) CD8⁺ CAR-T cells (5×10⁶) transduced with control sgRNA (Ametrine⁺) (n=5 recipients) or sgRegnase-1 (Ametrine⁺) (n=5 recipients) were transferred into mice at day 7 after Ph⁺B-ALL cell engraftment, followed by analyses of mouse survival (FIG. 2E) and tumor burden via Xenogen imaging of bioluminescent signal intensities (FIG. 2F). Non-treatment control group mice received no T cell transfer (n=5 recipients). NS, not significant; *P<0.05; **P<0.01; ***P<0.001; two-way ANOVA in FIGS. 2A, 2C, and Log-rank (Mantel-Cox) test in FIGS. 2B, 2D, and 2E. Data are representative of two (FIGS. 2A, AB, 2E, 2F) or four (FIGS. 2C, 2D) independent experiments.

FIGS. 3A-3M demonstrate that deletion of Regnase-1 reprograms tumor-infiltrating effector CD8⁺ T cells to acquire better persistence capacity while retaining robust effector function. (FIG. 3A) GSEA enrichment plots of sgRegnase-1- transduced OT-I cells isolated from TILs, using gene sets of antigen-specific CXCR5⁺and CXCR5⁻ exhausted CD8⁺ T cells from chronic infection and hematopoietic stem cells. (FIG. 3B) Control sgRNA-(mCherry⁺) and sgRegnase-1-transduced OT-I cells (Ametrine⁺) were mixed and transferred into tumor-bearing mice (n=5 mice), and tumor-infiltrating OT-I T cells were analyzed at day 7 for expression of CD27, CD43 activation-associated glycoform (CD43a) (left), and quantification of mean fluorescence intensity (MFI; numbers above graphs) of CD27 and CD43a (right). (FIGS. 3C-3D) OT-I cells transduced with control sgRNA (mCherry⁺) and sgRegnase-1 (Ametrine⁺) were mixed and transferred into tumor-bearing mice (n=5 mice), and tumor-infiltrating OT-I T cells were analyzed at day 14 for BrdU incorporation (FIG. 3C, left; pulse for 18 h) and active caspase-3 expression (FIG. 3D, left), and quantification of frequencies of BrdU⁺(FIG. 3C, right) and active caspase-3⁺ cells (FIG. 3D, right). Numbers in graphs indicate frequencies of cells in gates. (FIGS. 3E-3G) T cell in vivo persistence assays. Diagram of in vivo persistence assay (FIG. 3E): OT-I cells transduced with control sgRNA (GFP⁺) (1×10⁵) and sgRegnase-1 (Ametrine⁺) (1×10⁵) were isolated from TILs at 7 days after adoptive transfer, and then mixed at a 1:1 ratio (1×10⁵ each cell population) and transferred into tumor-bearing hosts (FIG. 3F) or naive mice (FIG. 3G). Flow cytometry analysis of OT-I cells in the TILs of CD8α⁺ cells in tumor-bearing hosts at days 3 (n=6 mice) and 7 (n=6 mice) after adoptive transfer (FIG. 3F, upper) or in the spleen of naive hosts at days 7 (n=6 mice) and 14 (n=6 mice) after adoptive transfer (FIG. 3G, upper), and quantification of normalized OT-I cell frequency in the TILs of tumor-bearing hosts (FIG. 3G, lower) or in the spleen of na-ve host (FIG. 3G, lower). Numbers in graphs indicate frequencies of cells in gates. (FIGS. 3H-3J) Control sgRNA-(mCherry⁺) and sgRegnase-1-transduced OT-I cells (Ametrine⁺) were mixed at 5:1 ratio and transferred into tumor-bearing mice, followed by analyses at days 7 (n=10 mice) or 14 (n=10 mice). Flow cytometry analysis of expression of IFN-γ (FIG. 3H, upper) and GzmB (FIG. 3H, lower) in TIL OT-I cells, and quantification of frequency of IFN-γ⁺ cells and Gzml3⁺ cells (FIG. 31) in TILs, and MFI of IFN-γ in IFN-γ⁺ cells and MFI of GzmB in Gzml3⁺ cells (FIG. 3J) in TILs. Numbers adjacent to outlined areas indicate frequency of IFN-γ⁺ cells and MFI of IFN-γ in IFN-γ⁺ cells (FIG. 3H, upper), and frequency of Gzml3⁺ cells and MFI of GzmB in Gzml3⁺ cells (FIG. 3H, lower). (FIG. 3K) OT-I cells transduced with control sgRNA (mCherry⁺) and sgRegnase-1 (Ametrine⁺) were mixed and transferred into tumor-bearing mice (n=5 mice), and tumor-infiltrating OT-I cells were analyzed at day 7 for expression of TCF-1 (left), and quantification of frequency of TCF-1⁺ cells (right). Numbers in graphs indicate frequencies of cells in gates. (FIGS. 3L, 3M) scRNA-Seq analysis of control sgRNA- and sgRegnase-1-transduced OT-I cells isolated from TILs. Specifically, control sgRNA- and sgRegnase-1-transduced OT-I cells were mixed and transferred into tumor-bearing mice, and tumor-infiltrating OT-I cells were isolated at day 7 for transcriptional profiling by scRNA-Seq. tSNE visualization of control sgRNA- and sgRegnase-1-transduced cells indicating genotypes (FIG. 3L, left), Tcf7^{hi} and Tcf7^{lo} cells (FIG. 3L, right; numbers in parentheses indicate cell numbers of each subset), and Tcf7 (FIG. 3M, left) and Slamf6 (FIG. 3M, right) gene expression in individual cells. Tcf7^{h1} cells were defined with the highest third quantile of Tcf7 expression (with log₂ gene expression intensity =2.910317 as threshold) among all cells. Mean±s.e.m. in FIGS. 3B-3D, 3F, 3G, 3I, 3J, and 3K. *P<0.05; **P<0.01; ***P<0.001; two-tailed unpaired Student's t-test in FIGS. 3B-3D, 3F, 3G and 3K; two-tailed paired Student's t-test in FIGS. 3I and 3J. Data are representative of one (FIGS. 3A, 3L, and 3M) or two (FIGS. 3B and 3I) or three (FIG. 3K) independent experiments, or pooled from two (FIGS. 3C, 3D, 3F, 3G, 3I, and 3J) independent experiments.

FIGS. 4A-4U illustrate the identification of BATF as a key Regnase-1 functional target as well as PTPN2 and SOCS1 as additional modifiers using genome-scale CRISPR-Cas9 screening. (FIG. 4A) Diagram of genome-scale screening system. Naïve Cas9-expressing OT-I cells were co-transduced with lentiviral sgRegnase-1 and genome-scale sgRNA library and expanded in vitro before transfer into tumor-bearing mice. OT-I cells were purified from TILs at 7 days after adoptive transfer, and library representation in TILs and pre-transfer (input) OT-I cells was examined by deep sequencing of sgRNA cassette. (FIG. 4B) OT-I cells transduced with control sgRNA (mCherry⁺) and sgRegnase-1 (Ametrine⁺) were mixed and transferred into tumor-bearing mice (n=6 mice), and tumor-infiltrating OT-I cells were analyzed at day 7 for TMRM and Mitotracker staining (FIG. 4B, upper), and quantification of MFI of TMRM and Mitotracker (FIG. 4B, lower). Numbers in graphs indicate MFI of TMRM and Mitotracker (FIG. 4B, upper). (FIG. 4C) Oxygen consumption rate (OCR) bioenergetics profiling of control sgRNA- and sgRegnase-1-transduced OT-I cells cultured in vitro for basal (left) and maximal OCR (right). (FIG. 4D) OT-I cells transduced with control sgRNA (mCherry⁺) and sgRegnase-1 (Ametrine⁺) were mixed and transferred into tumor-bearing mice (n=6 mice), and tumor-infiltrating OT-I cells were analyzed at day 7 for BATF (left) expression, and quantification of BATF MFI (right). Numbers in graphs indicate MFI of BATF. (FIG. 4E) Summary of ATAC-Seq motif enrichment data showing log₂ (odds ratio) and logio (FDR) of cells from control- (n=4 samples) and sgRegnase-1-transduced OT-I cells (n=4 samples) isolated from TILs at 7 days after adoptive transfer. (FIG. 4F) Luciferase activity of HEK293T cells measured at 48 h after transfection with Batf mRNA 3′ UTR luciferase reporter, together with control (mock), wild-type or D141N Regnase-1-expressing plasmid (n=3 samples each group). (FIG. 4G-4I) In vivo accumulation of double sgRNA-transduced OT-I cells in tumor-bearing mice. Specifically, OT-I cells transduced with sgRegnase-1 (mCherry⁺) were mixed at 1:1 ratio with cells transduced with control sgRNA (Ametrine⁺; upper), sgRegnase-1 (Ametrine⁺; middle) or sgRegnase-1 and sgBatf (Ametrine⁺ and GFP⁺ respectively; lower), and transferred into tumor-bearing hosts (n=5 mice each group) (FIG. 4G). Similar transfer system was used in (FIG. 4H) (n=5 mice each group) and (FIG. 41) (n=5 mice each group), except that sgPtpn2 and sgSocs1, respectively, were used in lieu of sgBatf (A second sgRNA, targeting Batf, Ptpn2 or Socs1, was examined in FIGS. 12D and 13A). Mice were analyzed at 7 days after adoptive transfer for proportion of OT-I cells in CD8α⁺ cells (FIGS. 4G-4I, left), and quantification of relative OT-I cell percentage in CD8α⁺ cells normalized to input in the spleen (FIGS. 4G-4I, upper right) and TILs (FIGS. 4G-4I, lower right). Numbers in plots indicate frequencies of OT-I cells (FIGS. 4G-4I, left). (FIG. 4J) In vivo accumulation of double sgRNA-transduced OT-I cells in tumor-bearing mice. Specifically, OT-I cells transduced with control sgRNA (mCherry⁺; spike) were mixed at a 1:1 ratio with cells transduced with control sgRNA (Ametrine⁺), sgRegnase-1 (Ametrine⁺), sgBatf (GFP⁺) or sgBatf/Regnase-1 (GFP⁺ and Ametrine⁺), and transferred into tumor-bearing hosts individually (n=6 mice each group). Mice were analyzed at 7 days after adoptive transfer for quantification of relative OT-I cell percentage in CD8α⁺ cells normalized to spike in the spleen (left) and TILs (right). (FIG. 4K) In vivo accumulation of BATF-overexpressing OT-I cells in tumor-bearing mice. Specifically, OT-I cells transduced with control retrovirus (mCherry⁺) were mixed at a 1:1 ratio with cells transduced with Batf-overexpressing retrovirus (GFP⁺), and transferred into tumor-bearing hosts (n=6 mice each group). Mice were analyzed at days 7 and 14 after adoptive transfer for quantification of relative OT-I cell percentage in CD8α⁺ cells normalized to input in TILs. (FIG. 4L) Principal component analysis (PCA) of transcriptomes. OT-I cells transduced with control sgRNA (mCherry⁺) (n=7 samples), sgRegnase-1 (Ametrine⁺) (n=4 samples), sgBatf (GFP⁺) (n=3 samples) or sgBatf/Regnase-1 (GFP⁺and Ametrine⁺) (n=4 samples), and transferred into tumor-bearing hosts individually. OT-I cells were isolated from TILs at day 7 for transcriptional profiling by microarray. (FIG. 4M) The same transfer system as in (FIG. 4J) was used (n=6 mice each group). Tumor-infiltrating OT-I cells were analyzed at day 7 for quantification of relative MFI of TMRM (left) and Mitotracker (right) normalized to spike. (FIG. 4N) The same transfer system as in (FIG. 4K) was used (n=8 mice each group). Tumor-infiltrating OT-I cells were analyzed at day 7 for quantification of MFI of TMRM (left) and Mitotracker (right). (FIGS. 4O-4S) OT-I cells transduced with control sgRNA (mCherry⁺; spike) were mixed at a 1:1 ratio with cells transduced with control sgRNA (Ametrine⁺), sgRegnase-1 (Ametrine⁺), sgPtpn2 (GFP⁺), sgPipn2/Regnase-1 (GFP⁺ and Ametrine⁺), sgSocs1 (GFP⁺) or sgSocs1/Regnase-1 (GFP⁺and Ametrine⁺), and transferred into tumor-bearing hosts individually (n=6 mice each group). Tumor-infiltrating OT-I cells were analyzed at day 7 for quantification of relative OT-I cell percentage in CD8α⁺ cells normalized to spike (FIG. 40), quantification of relative MFI of TMRM (FIG. 4P), Mitotracker (FIG. 4Q) and BATF (FIG. 4R) normalized to spike, and quantification of relative frequency of TCF-1⁺ cells normalized to spike (FIG. 4S). (FIG. 4T) Principal component analysis (PCA) of transcriptomes to compare Rengase-1-null, PTPN2-null and SOCS1-null cells. OT-I cells transduced with control sgRNA (Ametrine⁺), sgRegnase-1 (Ametrine⁺), sgPtpn2 (GFP⁺), sgPtpn2/Regnase-1 (GFP⁺and Ametrine⁺), sgSocs1 (GFP⁺) or sgSocs1/Regnase-1 (GFP⁺and Ametrine⁺) (n=4 samples each group) were transferred into tumor-bearing hosts individually. OT-I cells were isolated from TILs at day 7 for transcriptional profiling by microarray. (FIG. 4U) 4×10⁶ pmel-1 cells transduced with control sgRegnase-1 (Ametrine⁺), sgPtpn2/Regnase-1 (GFP⁺and Ametrine⁺), or sgSocs1/Regnase-1 (GFP⁺and Ametrine⁺) (n=10 recipients each group) were transferred into mice at day 12 after B16-F10 melanoma engraftment, followed by analysis of tumor size. Mean±s.e.m. in FIGS. 4B-4D, 4G-4I, 4J, 4K, 4M-4S. Mean±s.d. in FIG. 4F. NS, not significant; *P<0.05; **P<0.01; ***P<0.001; two-tailed paired Student's t-test in FIG. 4B, two-tailed unpaired Student's t-test in FIGS. 4C, 4D, 4F-4I, 4K, and 4 one-way ANOVA in FIGS. 4F, 4J, 4M, and O-S, and two-way ANOVA in FIG. 4U. Data are representative of one (FIG. 4E, 4L) or two (FIGS. 4C, and 4F-4I) independent experiments, or pooled from two (FIGS. 4B 4D, 4K, 4M-4S, and 4U) independent experiments.

FIGS. 5A-5E illustrate the validation of the effect of Regnase-1 in CDS⁺ T cell accumulation in tumor immunity using the in vivo dual transfer system. (FIG. 5A) Gating strategy for sgRNA-transduced OT-I cells. (FIGS. 5B-5C) OT-I cells transduced with control sgRNA (mCherry⁺) were mixed at a 1:1 ratio with either cells transduced with control sgRNA (Ametrine⁺) (FIGS. 5B-5C, upper left) or two different sgRNAs targeting Regnase-1 (sgRegnase-1, Ametrine⁺; FIG. 5B, lower left; or sgRegnase-1 #2, Ametrine⁺; FIG. 5C, lower left), and transferred into tumor-bearing hosts. (n=2-5 mice). Mice were analyzed at 7 days after adoptive transfer for analysis of the proportion of OT-I cells in CD8α⁺ cells (FIGS. 5B-5C, left), and quantification of relative OT-I cell percentage in CD8α⁺ cells normalized to input in the spleen and TILs (FIGS. 5B-5C, right). Numbers in plots indicate frequencies of OT-I cells. (FIG. 5D) OT-I cells transduced with control sgRNA (Ametrine⁺) were mixed at a 1:1 ratio with cells transduced with sgRegnase-1 (mCherry⁺), and transferred into tumor-bearing hosts (n=5 mice). Mice were analyzed at 7 days after adoptive transfer for analysis of the proportion of OT-I cells in CD8α⁺ cells (left), and quantification of relative OT-I cell percentage in CD8α⁺ cells normalized to input in the spleen and TILs (right). Numbers in plots indicate frequencies of OT-I cells. (FIG. 5E) Indel mutations after CRISPR-Cas9 targeted disruption in the in vitro cultured OT-I cells transduced with either control gRNA or sgRegnase-1, via deep sequencing analysis of indels generated at the exonic target site of Regnase-1 gene. Mean±s.e.m. in FIGS. 5B-5D. *P<0.05; ***P<0.001; two-tailed unpaired Student's t-test in FIGS. 5B-5D. Data are representative of two (FIG. 5D) independent experiments

FIG. 6 shows the antitumor efficacy of Regnase-1-deficient CDS⁺ CAR-T cells. Xenogen images of bioluminescent intensities of mice received CDS⁺ CAR-T cell therapy are presented. CDS⁺ CAR-T cells (5×10⁶) transduced with control sgRNA or sgRegnase-1 (n=5 recipients each group) were transferred into mice at day 7 after Ph⁺B-ALL cell engraftment. Non-treatment control group mice received no T cell transfer (n=5 recipients). Data are representative of two independent experiments.

FIGS. 7A-7H demonstrate that tumor-infiltrating and peripheral Regnase-1-null CD8⁺ T cells show distinct immune signatures. (FIGS. 7A-7B) GSEA enrichment plots of antigen-specific CXCR5⁺and CXCR5⁻ exhausted CD8⁺ T cells from chronic infection using gene targets repressed by Regnase-1 (i.e. top 100 upregulated genes in TIL sgRegnase-1-compared to control sgRNA-transduced OT-I cells as identified by bulk RNA-Seq). (FIG. 7C) Venn diagram showing the overlap of significantly upregulated (left, sgRegnase-1- (n=5 samples) versus control sgRNA-transduced OT-I cells (n=4 samples)) or downregulated genes (right, sgRegnase-1- versus control sgRNA-transduced OT-I cells) by bulk RNA-Seq profiling between TIL and PLN OT-I cells. Specifically, control sgRNA- and sgRegnase-1-transduced OT-I cells were mixed and transferred into tumor-bearing mice, and OT-I cells were isolated at day 7 for transcriptional profiling by RNA-Seq. (FIGS. 7D-7E) List of the top 10 significantly (FDR <0.05) upregulated and downregulated pathways in TIL sgRegnase-1-versus control sgRNA-transduced OT-I cells (FIG. 7D) and PLN sgRegnase-1-versus control sgRNA-transduced OT-I cells (FIG. 7E), as revealed by performing GSEA using “immunologic signatures” gene sets. (FIGS. 7F-7G) GSEA enrichment plots of TIL sgRegnase-1- versus control sgRNA-transduced OT-I cells (FIG. 7F) and PLN sgRegnase-1-versus control sgRNA-transduced OT-I cells (FIG. 7G) using gene sets of four different tumor-infiltrating CD8 T cell activation states. Specifically, control sgRNA- and sgRegnase-1-transduced OT-I cells were mixed and transferred into tumor-bearing mice, and PLN OT-I cells were isolated at day 7 for transcriptional profiling by RNA-Seq. (FIG. 7H) OT-I cells transduced with control sgRNA (mCherry⁺) and sgRegnase-1 (Ametrine⁺) were mixed and transferred into tumor-bearing mice (n=5 mice), and OT-I cells in the spleen were analyzed at day 7 for expression of TCF-1 (left), and quantification of frequency of TCF-1⁺ cells (right). Numbers in graphs indicate frequencies of cells in gates.

FIGS. 8A-8F show the altered transcriptional programs and chromatin accessibility of TIL Regnase-1-null CD8⁺ T cells. (FIG. 8A) Gene expression heat maps normalized by row (z-score) for the na-ve or memory T cell-associated transcription factors in control sgRNA- and sgRegnase-1-transduced OT-I cells isolated from TILs. (FIG. 8B) Representative images (left) and quantification of MFI (right) of TCF-1 expression (pink) in control sgRNA-(mCherry⁺; red) and sgRegnase-1-transduced OT-I cells (Ametrine⁺; green) in the whole tumor section (n=4 mice). Scale bars, 20 p.m. (FIG. 8C) Gene expression heat maps normalized by row (z-score) for the effector or exhausted T cell-associated transcription factors in control sgRNA- and sgRegnase-1-transduced OT-I cells isolated from TILs. Specifically, control sgRNA- and sgRegnase-1-transduced OT-I cells were mixed and transferred into tumor-bearing mice, and tumor-infiltrating OT-I cells were isolated at day 7 for transcriptional profiling by RNA-Seq. (FIG. 8D) Real-time PCR analysis of Irf4 mRNA expression in control sgRNA- (n=4 samples) and sgRegnase-1-transduced OT-I cells (n=5 samples) isolated from TILs. (FIG. 8E) Summary of ATAC-Seq motif enrichment data showing log₂ (odds ratio) and logio (FDR) of cells from control sgRNA- (n=4 samples) and sgRegnase-1-transduced OT-I cells (n=4 samples) isolated from TILs. Specifically, control sgRNA- and sgRegnase-1-transduced OT-I cells were mixed and transferred into tumor-bearing mice, and tumor-infiltrating OT-I cells were isolated at day 7 for ATAC-Seq analysis. (FIG. 8F) Tn5 insert sites from ATAC-Seq analysis were aligned to motifs for transcription factors from the TRANSFAC database, and the binding profiles of TCF-1, Bach2, Bc16 and IRF4 are shown. Mean±s.e.m. in FIGS. 8B and 8D. *P<0.05; **P<0.01; two-tailed unpaired Student's t-test in FIGS. 8B and 8D. Data are representative of one (FIGS. 8A, 8C, 8E, and 8F) or two (FIGS. 8B and 8D) independent experiments.

FIGS. 9A-9N show the proliferation and survival analyses of Regnase-1-null CD8⁺ T cells in tumor immunity. (FIG. 9A) List of the top 10 significantly (FDR <0.05) upregulated and downregulated pathways in TIL sgRegnase-1-transduced OT-I cells, as revealed by performing GSEA using “Hallmark” gene sets. Specifically, control sgRNA- and sgRegnase-1-transduced OT-I cells were mixed and transferred into tumor-bearing mice, and tumor-infiltrating OT-I cells were isolated at day 7 for transcriptional profiling by RNA-Seq. (FIG. 9B) GSEA enrichment plots of TIL sgRegnase-1-transduced OT-I cells using cell cycling-associated gene sets, including E2F targets (left), G2M checkpoint (middle) and mitotic spindle (right). (FIGS. 9C-9E) OT-I cells transduced with control sgRNA (mCherry⁺) and sgRegnase-1 (Ametrine⁺) were mixed and transferred into tumor-bearing mice, and tumor-infiltrating OT-I cells were analyzed at day 7 (FIGS. 9D and 9E) (n=6 mice) and day 14 (FIG. 9C) (n=5 mice) for flow cytometry analysis of Ki-67 expression (FIGS. 9C, left; 9E, upper) and BrdU incorporation (FIG. 9D, upper; pulse for 18 h), and quantification of MFI of Ki-67 (FIGS. 9C, right; 9E, lower) and frequency of BrdU⁺ cells (FIG. 9D, lower). Numbers in graphs indicate MFI of Ki-67 (FIGS. 9C, left; 9E, upper). Numbers in plots indicate frequencies of BrdU⁺ cells (FIG. 9D, upper). (FIG. 9F) GSEA enrichment plots of TIL sgRegnase-1-transduced OT-I cells using apoptosis gene set. (FIG. 9G) Gene expression heat maps normalized by row (z-score) for anti-apoptotic Bcl2l1 (encodes Bc1-xL) and pro-apoptotic Bcl2l11 (encodes Bim) in control sgRNA- and sgRegnase-1-transduced OT-I cells isolated from TILs. (FIG. 9I1) Real-time PCR analysis of Bcl2l1 mRNA expression in control sgRNA- (n=4 samples) and sgRegnase-1-transduced OT-I cells (n=5 samples) isolated from TILs. (FIGS. 91 and 9J) Control sgRNA (mCherry⁺) and sgRegnase-1-transduced OT-I cells (Ametrine⁺) were mixed and transferred into tumor-bearing mice (n=6 mice), and tumor-infiltrating OT-I cells were analyzed at day 7 for Bim expression (FIG. 9I, left) and the active caspase-3 expression (FIG. 9J, left), and quantification of MFI of Bim (FIG. 9I, right) and frequency of active caspase-3⁺ cells (FIG. 9J, right). Numbers in graphs indicate MFI of Ki-67 (FIG. 9I, left). Numbers in plots indicate frequencies of active caspase-3⁺ cells (FIG. 9J, left). (FIG. 9K) List of the top 15 significantly (FDR<0.05) upregulated and top 4 significantly downregulated pathways in PLN sgRegnase-1-transduced OT-I cells, as revealed by performing GSEA using “Hallmark” gene sets. (FIGS. 9L and 9M) Control sgRNA-(mCherry⁺) and sgRegnase-1-transduced OT-I cells (Ametrine⁺) were mixed and transferred into tumor-bearing mice (n=6 mice), and splenic OT-I cells were analyzed for BrdU incorporation (FIG. 9L, upper; pulse for 18 h) and the active caspase-3 expression (FIG. 9M, upper), and quantification of frequency of BrdU⁺ cells (FIG. 9L, lower) and active caspase-3⁺ cells (FIG. 9M, lower). Numbers in plots indicate frequencies of BrdU⁺ cells (FIG. 9L, upper) or active caspase-3⁺ cells (FIG. 9M, upper). (FIG. 9N) OT-I cells transduced with control sgRNA (mCherry⁺) and sgRegnase-1 (Ametrine⁺) were mixed and transferred into tumor-bearing mice, and tumor-infiltrating OT-I cells were analyzed at day 7 (n=6 mice) for flow cytometry analysis of a DNA damage marker Ser139 phosphorylation of histone variant H2A.X (FIG. 9N, upper) and quantification of frequency of Ser139 phosphorylated histone variant H2A.X⁺ cells (FIG. 9N, lower). Numbers in plots indicate frequencies of Ser139 phosphorylated histone variant H2A.X⁺ cells (FIG. 9N, upper). Numbers in graphs indicate frequencies of Ser139 phosphorylated histone variant H2A.X⁺ cells (FIG. 9N, upper). Mean±s.e.m. in FIGS. 9C-9E, 9H-9J, 9L, 9M, and 9N. *P<0.05; **P<0.01; ***P<0.001; and two-tailed unpaired Student's t-test in FIGS. 9C-9E, 9H-9J, 9L, and 9M. Data are representative of one (FIGS. 9A, 9B, 9F, 9G, and 9K) or two (FIG. 9C) independent experiments, or pooled from two (FIGS. 9D, 9E, 9I, 9J, 9L, 9M and 9N) independent experiments.

FIGS. 10A-10G show the effector molecular expression of tumor-infiltrating Regnase-1-null CD8⁺ T cells. (FIGS. 10A-10B) Control sgRNA-(mCherry⁺) and sgRegnase-1-transduced OT-I cells (Ametrine⁺) were mixed at 5:1 ratio and transferred into tumor-bearing mice (n=5 mice), and tumor-infiltrating OT-I cells were analyzed at day 7 for the expression of CD69, CD25, CD49a, CXCR3, KLRG1, ICOS, Tim3, Lag3, PD-1 and CTLA4 (FIG. 10A, upper) and CD44 and CD62L (FIG. 10B, upper), and quantification of MFI of CD69, CD25, CD49a, CXCR3, KLRG1, ICOS, Tim3, Lag3, PD-1 and CTLA4 (FIG. 10A, lower) and frequency of CD44⁺ CD62L⁻ cells (FIG. 10B, lower). Numbers in graphs indicate MFI (FIG. 10A, upper). Numbers in plots indicate frequency of CD44⁺ CD62L⁻ cells (FIG. 10B, upper). (FIGS. 10C-10G) Control sgRNA (mCherry⁺) and sgRegnase-1-transduced OT-I cells (Ametrine⁺) were mixed at 5:1 ratio and transferred into tumor-bearing mice, and analyzed at days 7 (n=10 mice) or 14 (n=10 mice) for the number of IFN-γ⁺ cells or GzmB⁺ cells normalized to input per gram tissue (FIG. 10C), expression of TNF-α (FIG. 10D, upper) and IL-2 (FIG. 10D, lower) in TIL OT-I cells, and quantification of frequencies of TNF-α⁺ cells and IL-2⁺ cells (FIG. 10E) in TILs, and number of TNF-α⁺ cells or IL-2⁺ cells normalized to input per gram tissue (FIG. 10F) in tumors, and frequency (FIG. 10G, left) and number (normalized to input) per gram tissue (FIG. 10G, right) of polyfunctional IFN-γ⁺ TNF-α⁺IL-2⁺ cells in TILs. Numbers in plots indicate frequencies of TNF-α⁺ cells (FIG. 10D, upper), or IL-2⁺ cells (FIG. 10D, lower). Mean±s.e.m. in FIGS. 10A-10C and 10E-10G. NS, not significant; *P<0.05; **P<0.01; ***P<0.001; two-tailed unpaired Student's t-test in FIGS. 10A-10B, and two-tailed paired Student's t-test in FIGS. 10C and 10E-10G. Data are representative of two (FIGS. 10A, 10B, and 10D) independent experiments, or pooled from two (FIGS. 10C and 10E-10G) independent experiments.

FIGS. 11A-11D illustrate the identification of immune regulators and OXPHOS metabolic pathway using genome-scale CRISPR-Cas9 screening. (FIG. 11A) Scatterplot of the enrichment of each gene versus its adjusted P values in genome-scale CRISPR-Cas9 screening. Gene enrichment was calculated by averaging the enrichment of their 4 sgRNAs in tumor-infiltrating OT-I cells relative to input (log₂ ratio (TIL/input)), with the most extensively enriched (dark solid circle) and selective depleted (stripe-filled circle) genes (adjusted P<0.05), as well as ‘dummy’ genes (empty circle; generated by random combinations of 4 out of 1,000 non-targeting control sgRNAs per ‘dummy’ gene) highlighted. (FIG. 11B) Functional enrichment plots of the top 10 significantly (FDR <0.05) enriched pathways in top-ranking depleted genes identified in the genome-scale CRISPR-Cas9 screening (by less than -3.5 log₂ (TIL/input) fold change; adjusted P<0.05). (FIG. 11C) GSEA enrichment plots of TIL sgRegnase-1-transduced OT-I cells using OXPHOS gene set. (FIG. 11D) Representative images (left) and quantification of mitochondrial volume (stained with Tom20, white) per cell (right) in control sgRNA-(mCherry⁺; red) and sgRegnase-1 transduced OT-I cells (Ametrine⁺; green) in tumors at 7 days after adoptive transfer (n=4 mice). Mean±s.e.m. in FIG. 11D. *P<0.05; two-tailed unpaired Student's t-test in FIG. 11D. Data are representative of one (FIGS. 11A-11C) or two (FIG. 11D) independent experiments.

FIGS. 12A-12L demonstrates that BATF is a key Regnase-1 target in tumor immunity and regulates mitochondrial function. (FIG. 12A) Venn diagram showing the overlap of genes between top depleted genes in genome-scale CRISPR-Cas9 screening (by less than −3.5 log₂ (TIL/input) fold change; adjusted P<0.05) and top upregulated genes in TIL sgRegnase-1- versus control sgRNA-transduced OT-I cells as identified by bulk RNA-Seq (by greater than 1.5 log₂ fold change; P<0.05). (FIG. 12B) Tn5 insert sites from ATAC-Seq analysis were aligned to motifs for transcription factors from the TRANSFAC database, and the binding profiles of BATF are shown. (FIG. 12C) Luciferase activity of HEK293T cells measured at 48 h after transfection with Il2 mRNA 3′ UTR (upper) or Il4 mRNA 3′ UTR (lower) luciferase reporter plasmid, together with control (mock), wild-type or D141N Regnase-1-expressing plasmid (n=3 samples each group). (FIG. 12D) In vivo accumulation of double sgRNA-transduced OT-I cells in tumor-bearing mice, similar as FIG. 4G, for the use of the second sgRNA targeting Batf (see FIG. 4G legend for details). (FIG. 12E) Flow cytometry analysis of BATF expression in control sgRNA, sgRegnase-1, sgBatf and sgBatf and sgRegnase-1 co-transduced OT-I cells cultured in vitro and stimulated with anti-CD3 and anti-CD28 for indicated time. Numbers in graphs indicate MFI of BATF and are listed in the same order as the legend. (FIG. 12F) OT-I cells transduced with control sgRNA (mCherry⁺) were mixed with cells transduced with sgBatf (GFP⁺), and transferred into tumor-bearing hosts (n=5 mice). Mice were analyzed at 7 days after adoptive transfer for analysis of the proportion of OT-I cells in CD8α⁺ cells (left), and quantification of relative OT-I cell percentage in CD8α⁺ cells normalized to input in the spleen and TILs (right). Numbers in plots indicate frequencies of OT-I cells. (FIG. 12G) OT-I cells transduced with sgRegnase-1 (mCherry⁺) were mixed with cells co-transduced with sgRegnase-1 and sgBatf (Ametrine⁺and GFP⁺, respectively) and transferred into tumor-bearing hosts (n=3 mice), and TILs were analyzed at 7 days after adoptive transfer for CD44 and CD62L expression (left), and quantification of the frequency of CD44⁺ CD62L⁻ cells (right) in OT-I cells. Numbers in plots indicate frequencies of CD44⁺ CD62L⁻ cells. (FIG. 12I1) Gene expression heat maps normalized by row (z-score) for Ifng, Gzmb and Gzma expression in sgRegnase-1-transduced and sgBatf and sgRegnase-1 co-transduced OT-I cells isolated from TILs. (FIG. 12I) Real-time PCR analysis of Ifng, Gzmb and Gzma mRNA expression in sgRegnase-1-transduced and sgBatf and sgRegnase-1 co-transduced OT-I cells isolated from TILs (n=4 mice each group). (FIG. 12J) List of the top 2 significantly (FDR<0.05) upregulated and top 8 significantly downregulated pathways in TIL sgBatf and sgRegnase-1 co-transduced (n=3 samples) versus sgRegnase-1-transduced (n=3 samples) OT-I cells isolated from TILs, as revealed by performing GSEA using “Hallmark” gene sets. (FIG. 12K) GSEA enrichment plots of TIL sgBatf and sgRegnase-1 co-transduced OT-I cells using OXPHOS gene set. (FIG. 12L) OT-I cells transduced with sgRegnase-1 (Ametrine⁺) were mixed with cells co-transduced with sgRegnase-1 and sgBatf (Ametrine⁺and GFP⁺, respectively) and transferred into the tumor-bearing mice (n=6 mice), and tumor-infiltrating OT-I cells were analyzed at day 7 for TMRM and Mitotracker staining (left), and quantification of the MFI of TMRM and Mitotracker (right) in OT-I cells. Numbers in graphs indicate MFI of TMRM and Mitotracker. Mean±s.e.m. in FIGS. 12D, 12F, 12G, 12I, and 12L, and mean±s. d. in FIG. 12C. NS, not significant; *P<0.05; **P<0.01; ***P<0.001; two-tailed unpaired Student's t-test in FIGS. 12C and 12F and two-tailed paired Student's t-test in FIGS. 12G, 12I, and 12L. Data are representative of one (FIGS. 12B, 12D, 12H, 12J, and 12K) or two (FIGS. 12C, 12E, and 12G) independent experiments, or pooled from two (FIGS. 12F and 12L) independent experiments.

FIGS. 13A-13E illustrate the identification of additional targets for ACT in cancer immunotherapy using genome-scale CRISPR-Cas9 screening. (FIG. 13A) In vivo accumulation of double sgRNA-transduced OT-I cells in tumor-bearing mice, similar as FIG. 4G, except for the use of the sgRNAs targeting Ptpn2, Socs 1 and Agps (see FIG. 4G legend for details). (FIGS. 13B and 13C) OT-I cells transduced with control sgRNA (mCherry⁺) were mixed with either cells transduced with sgPtpn2 (GFP⁺) (n=3 mice) (FIG. 13B, upper), sgSocs1 (GFP⁺) (n=5 mice) (FIG. 13B, lower) or sgRoquin-1 (sgRc3h1) (GFP⁺) (n=3 mice) (FIG. 13C), and transferred into tumor-bearing hosts. Mice were analyzed at 7 days after adoptive transfer for analysis of the proportion of OT-I cells in CD8α⁺ cells (FIGS. 13B and 13C, left), and quantification of relative OT-I cell percentage in CD8α⁺ cells normalized to input in the spleen and TILs (FIG. 13B and 13C, right). Numbers in plots indicate frequencies of OT-I cells. (FIG. 13D) List of the top 9 significantly (FDR <0.05) upregulated and top 13 significantly downregulated pathways in sgPtpn2 and sgRegnase-1 co-transduced (n=3 samples) versus sgRegnase-1-transduced (n=3 samples) OT-I cells isolated from the TILs, as revealed by performing GSEA using “Hallmark” gene sets. (FIG. 13E) List of the top 10 significantly (FDR<0.05) upregulated and downregulated pathways in TIL sgSocs1 and sgRegnase-1 co-transduced (n=3 samples) versus sgRegnase-1-transduced (n=3 samples) OT-I cells isolated from the TILs, as revealed by performing GSEA using “Hallmark” gene sets. Mean±s.e.m. in FIGS. 13A-13C. *P<0.05; **P<0.01; two-tailed unpaired Student's t-test in FIGS. 13B and 13C. Data are representative of one (FIGS. 13A, 13C, 13D, and 13E) or pooled from two (FIGS. 13B) independent experiments.

FIG. 14 depicts a schematic of deletion of Regnase-1 reprograms CD8⁺ T cells for improved cancer immunotherapy. Regnase-1 is a major negative regulator of CD8⁺ T cell antitumor responses, and TCR and IL-2 inhibit its expression and activity. Deletion of Regnase-1 unleashes potent therapeutic efficacy of engineered tumor-specific CD8⁺ T cells against cancers by coordinating transcriptional and metabolic programs to achieve greatly improved cell accumulation and function. As a key functional target of Regnase-1, excessive BATF drives robust cell accumulation and effector function, in part through enhancing mitochondrial metabolism, in Regnase-1-null CD8⁺ T cells. Regnase-1 deletion also reprograms cells to acquire increased naive/memory cell-associated gene signatures and gain survival advantage, which contribute to the improved persistence of Regnase-1-null effector CD8⁺ T cells. Targeting PTPN2 and SOCS1 (not depicted here) acts in coordination with Regnase-1 inhibition to promote CD8⁺ T cell antitumor responses.

FIGS. 15A-15G demonstrate that upstream signals regulate Regnase-1 expression and Regnase-1-null cell phenotypes. (FIG. 15A) Immunoblot analysis of Regnase-1 expression in wild-type OT-I cells isolated from PLN and TILs at 7 days after adoptive transfer (n=4 samples each group) (upper). Quantification of relative intensity of Regnase-1 expression (lower). (FIG. 15B) GSEA enrichment plots of PLN and TIL control sgRNA-OT-I cells used in (FIG. 15A) by using gene targets repressed by Regnase-1 (i.e. top 100 upregulated genes in TIL sgRegnase-1- compared to control sgRNA-transduced cells as identified by RNA-Seq). (FIG. 15C) OT-I cells were stimulated with aCD3 and αCD28 for overnight before viral transduction, and then cultured in IL-7 and IL-15-containing medium for another 3 days in vitro. Pre-activated OT-I cells were then stimulated with aCD3, IL-2 or IL-21 for 0, 1 and 4 h (n=5 samples each group) for immunoblot analysis of full length and cleaved Regnase-1 (upper), and quantification of relative intensity of full length (lower left) and cleaved Regnase-1 expression (lower right). (FIGS. 15D, 15E) OT-I cells transduced with control sgRNA (mCherry⁺) and sgRegnase-1 (Ametrine⁺) were mixed at a 1:1 ratio and transferred into mice bearing B16-Ova (n=6 mice) or B16-F10 (n=6 mice) tumors. Mice were analyzed at day 7 after adoptive transfer for quantification of relative OT-I cell percentage in total cells normalized to input in the spleen and TILs (FIG. 15D), and expression of TCF-1 (FIG. 15E, left), and quantification of frequency of TCF-1⁺ cells (FIG. 15E, right) in tumor-infiltrating OT-I cells. (FIG. 15F, 15G) OT-I cells were stimulated with aCD3 and αCD28 for overnight before viral transduction, and then cultured in IL-2, IL-7 and IL-15-containing medium for another 3 days in vitro. Pre-activated OT-I cells were then continuously cultured in normoxia (21% O₂) or hypoxia (1% O₂) condition for 48 h for immunoblot analysis of expression of HIF1α, Regnase-1 and BATF (FIG. 15F), and for flow cytometry analysis of expression of BATF, CD69, GzmB, CD25 and TCF-1 (FIG. 15G). Numbers in graphs indicate MFI and appear in the same order as the legend (FIG. 15G). Mean±s.e.m. in FIGS. 15A, 15C-15E. *P<0.05; **P<0.01; ***P<0.001; two-tailed unpaired Student's t-test in FIG. 15A, and one-way ANOVA in FIGS. 15C-15E. Data are representative of two (FIGS. 15C, 15F, 15G) independent experiments, or pooled from two (FIGS. 15D, 15E) independent experiments.

FIGS. 16A-16G show scRNA-Seq and flow cytometry analyses of tumor-infiltrating Regnase-1-null OT-I cells. (FIGS. 16A-16F) scRNA-Seq analysis of control sgRNA- and sgRegnase-1-transduced OT-I cells isolated from TILs. Specifically, control sgRNA- and sgRegnase-1-transduced OT-I cells were mixed and transferred into tumor-bearing mice, and tumor-infiltrating OT-I cells were isolated at day 7 for transcriptional profiling by scRNA-Seq. tSNE visualization of Tox (FIG. 16A, left), Pdcdl (FIG. 16B, upper left), Havcr2 (FIG. 16B, lower left), Ifng (FIG. 16D, upper left), Gzmb (FIG. 16D, lower left), Batf (FIG. 16E, left) and Id2 (FIG. 16F, left) gene expression, and “CXCR5⁺ exhausted CD8 (Ahmed)⁷” (FIG. 16C, upper left) and “CXCR5⁺exhausted CD8 (Yu)⁸” (FIG. 16C, lower left) gene signatures in individual cells. Violin plots of Tox (FIG. 16A, right), Pdcd1 (FIG. 16B, upper right), Havcr2 (FIG. 16B, lower right), Ifng (FIG. 16D, upper right), Gzmb (FIG. 16D, lower right), Batf (FIG. 16E, right) and Id2 (FIG. 16F, right) gene expression, and “CXCR5⁺exhausted CD8 (Ahmed)” (FIG. 16C, upper right) and “CXCR5⁺exhausted CD8 (Yu)” (FIG. 16C, lower right) gene signatures among the four cell subsets. The black dots in the center of the violin plots indicate the median values. (FIG. 16G) OT-I cells transduced with control sgRNA and sgRegnase-1 were mixed and transferred into tumor-bearing mice (n=5 mice; data from one representative mouse were shown), and tumor-infiltrating OT-I cells were analyzed at day 7 for the expression of TOX, Slamf6, CD127, KLRG1, Tim3 and PD-1 in TCF-1⁺and TCF-1⁻ cells of control sgRNA- and sgRegnase-1-transduced OT-I cells. Numbers in graphs indicate mean±s.e.m. of MFI of markers on the X-axis after gating TCF-1⁺(control sgRNA: 15.5 ±2.9%; sgRegnase-1: 28.2±5.6%) and TCF-1⁻ subsets. Data are representative of one (FIGS. 16A-16F) or two (FIG. 16G) independent experiments.

FIGS. 17A-17H demonstrate that genome-scale CRISPR-Cas9 screening identifies BATF as an important Regnase-1 functional target in tumor immunity. (FIG. 17A) Enrichment of BATF-binding motifs in the genomic regions with upregulated accessibility in Regnase-1-null cells. First, common regions were analyzed in the Regnase-1-null ATAC-Seq data and published BATF ChIP-Seq peaks (GSE5419⁹). Next, these common regions with TRANSFAC motifs for BATF were scanned, and numbers of motif matches and associated Fisher's exact test P values and log₂ (odds ratios) are shown (a positive log₂ (odds ratio) value indicates that a motif is more likely to occur in Regnase-1-null cells than in wild-type samples; ‘E-x’ denotes ‘×10^{-x}’). (FIGS. 17B, 17C) OT-I cells transduced with control sgRNA (mCherry⁺; spike) were mixed at a 1:1 ratio with cells transduced with control sgRNA (Ametrine⁺), sgRegnase-1 (Ametrine⁺), sgBatf (GFP⁺) or sgBatf/Regnase-1 (GFP⁺and Ametrine⁺), and transferred into tumor-bearing hosts individually (n=4 mice each group). Mice were analyzed at 5 days after adoptive transfer for quantification of relative OT-I cell percentage in CD8α⁺ cells normalized to spike in the spleen (FIG. 17B, left) and TILs (FIG. 17B, right), and quantification of relative MFI of BATF normalized to spike in the tumor-infiltrating OT-I cells (FIG. 17C). (FIG. 17D) Immunoblot analysis of Regnase-1 and BATF expression in in vitro cultured OT-I cells 3 days after transduction with control sgRNA or sgBatflRegnase-1 . (FIGS. 17E-17G) The same transfer system as in (FIG. 17F) was used. Tumor-infiltrating OT-I cells were analyzed at day 5 (n=4 mice each group) for quantification of relative frequency of active caspase-3⁺ cells normalized to spike (FIG. 17E), and quantification of relative frequency of TCF-1⁺ cells normalized to spike (FIG. 17F), or at day 7 (n=6 mice each group) for quantification of relative frequency of IFN-γ⁺ cells normalized to spike (FIG. 17G). (FIG. 17H)4×10⁶ pmel-1 cells transduced with sgRegnase-1 (Ametrine⁺) (n=10 recipients) or sgBatf/Regnase-1 (GFP⁺and Ametrine⁺) (n=10 recipients) were transferred into mice at day 12 after B16-F10 melanoma engraftment, followed by analyses of tumor size. Mean±s.e.m. in FIGS. 17B, 17C, 17E-17G. NS, not significant; *P<0.05; **P<0.01; ***P<0.001; one-way ANOVA in FIGS. 17B-17C, 17E-17G, and two-way ANOVA in FIG. 17H. Data are representative of one (FIG. 17A) or three (FIG. 17D) independent experiments, or pooled from two (FIGS. 17B, 17C, 17E-17H) independent experiments.

FIGS. 18A-18H show BATF overexpression markedly enhances CD8⁺ T cell antitumor responses. (FIG. 18A) OT-I cells were stimulated with aCD3 and aCD28 for overnight before viral transduction, and then cultured in IL-7 and IL-15-containing medium for another 3 days in vitro. Control sgRNA- and sgRegnase-1-transduced OT-I cells were then stimulated with aCD3, IL-2 or IL-21 for overnight for flow cytometry analysis of BATF expression (upper), and quantification of the MFI of BATF (lower) (n=6 samples each group). Numbers in graphs indicate MFI (upper) and fold change between comparisons (lower). (FIG. 18B-18H) OT-I cells transduced with control retrovirus (mCherry⁺) were mixed at a 1:1 ratio with cells transduced with Batf-overexpressing retrovirus (GFP⁺), and transferred into tumor-bearing hosts. Mice were analyzed at day 4 (FIG. 18E) (n=4 mice), day 5 (FIGS. 18B, 18H) (n=4 mice), day 7 (FIGS. 18C, 18D, 18F, 18G) (n=6-8 mice) or day 14 (FIGS. 18C, 18D) (n=6 mice) for the expression of BATF (FIG. 18B, left), active caspase-3 (FIG. 18F, left), IFN-γ, GzmB, TNF-α and IL-2 (FIG. 18G, left), and TCF-1 (FIG. 18H, upper) in TIL OT-I cells, and quantification of MFI of BATF in TIL OT-I cells (FIG. 18B, right), and quantification of frequencies of active caspase-3⁺ cells (FIG. 18F, right), IFN-γ⁺, GzmB⁺, TNF-α⁺and IL-2⁺ cells (FIG. 18G, right), and TCF-1⁺ cells (FIG. 18H, lower) in TIL OT-I cells, and analysis of the proportion of donor-derived OT-I cells in total CD8α⁺ cells in TILs and spleen (FIG. 18C), and quantification of relative OT-I cell percentage in CD8α⁺ cells normalized to input in the spleen (FIG. 18D), and the dilution of CellTrace Violet (CTV) in TIL OT-I cells (FIG. 18E, left), and quantification of MFI of CTV in TIL OT-I cells (FIG. 18E, right). Numbers in graphs indicate MFI and appear in the same order as the legend (FIG. 18B, left; FIG. 18E, left), frequencies of OT-I cells in gates (FIGS. 18C), frequency of active caspase-3⁺ cells (FIG. 18F, left), frequencies of IFN-γ⁺, GzmB⁺, TNF-α⁺or IL-2⁺ cells (FIG. 18G, left), and frequency of TCF-1⁺ cells (FIG. 18H, upper). Mean±s.e.m. in FIGS. 18A, 18B, 18D-18H. *P<0.05; **P<0.01; ***P<0.001; two-tailed unpaired Student's t-test in FIGS. 18A, 18B, 18D-18H. Data are representative of two (FIGS. 18A, 18C) independent experiments, or pooled from two (FIGS. 18B, 18D-18H) independent experiments.

FIGS. 19A-19 D show ATAC-Seq and WCGNA analyses of wild-type, Regnase-1-null, BATF-null and BATF/Regnase-1-null cells. OT-I cells transduced with control sgRNA (mCherry⁺), sgRegnase-1 (Ametrine⁺), sgBatf (GFP⁺) or sgBatf/Regnase-1 (GFP⁺and Ametrine⁺) (n=2-4 samples each group) were transferred into tumor-bearing hosts individually. OT-I cells were isolated from TILs at day 7 for ATAC-Seq analysis (FIG. 19A) or transcriptional profiling by microarrays (FIGS. 19B, 19C). (FIG. 19A) Venn diagram depicting genes with differential chromatin accessibility (by |log₂ FC|>0.5; P<0.05) in sgRegnase-1-, sgBatf- or sgBatf/Regnase-1-transduced tumor-infiltrating OT-I cells. The differential accessibility (DA) regions were annotated in ATAC-Seq for the nearest genes. The numbers indicate the shared and independent genes in each category. (FIG. 19B) Weighted gene correlation network analysis (WGCNA) of control gRNA-, sgRegnase-1-, sgBatf-, and sgBatf/Regnase-1-transduced tumor-infiltrating OT-I cells. The number of genes in each cluster is indicated. Red dashed lines represent the relative gene expression level in control gRNA-transduced cells. Mitochondrial genes that were upregulated in the absence of Regnase-1 were shown in the corresponding clusters. (FIG. 19C) Functional enrichment of the clusters from WGCNA (FIG. 19B) using four tumor-infiltrating CD8⁺ T cell activation states^{1,2}. (FIG. 19D) Venn diagram depicting mitochondrial genes with differential chromatin accessibility (by |log2 FC|>0.5; P<0.05) in sgRegnase-1-, sgBatf- or sgBatf/Regnase-1-transduced tumor-infiltrating OT-I cells as determined by ATAC-Seq as described in FIG. 19A. The differential accessibility (DA) regions in ATAC-Seq were annotated for the nearest genes, and these genes were superimposed with 1,158 mitochondrial genes defined in MitoCarta 2.0 database^{ll}'¹². The numbers indicate the shared and independent genes in each category. Data are representative of one (FIGS. 19A-19 D) experiment.

FIGS. 20A-20B demonstrate PTPN2 and SOC S1 deletion efficiency and expression in Regnase-1-null cells. (FIG. 20A) Immunoblot analysis of Regnase-1, PTPN2 and SOCS1 expression in in vitro cultured OT-I cells 3 days after transduction with control sgRNA, sgPtpn2/Regnase-1 (left), or sgSocs1/Regnase-1 (right). (FIG. 20B) Immunoblot analysis of Regnase-1, BATF, SOCS1 and PTPN2 expression in control sgRNA- and sgRegnase-1-transduced OT-I cells cultured in vitro for 3 days after viral transduction. Data are representative of three (FIGS. 20A, 20B) independent experiments.

FIG. 21 shows a schematic of the six guide RNAs (gRNA1-gRNA6) selected in silico for knocking out Regnase-1 in human CAR-T cells.

FIGS. 22A-22B show Regnase-1 knockout confirmation in human T cells. FIG. 22A shows deep sequence results for three selected guide RNAs (gRNA1, gRNA2, and gRNA6). FIG. 22B is a Western blot showing the knock-down effect of three selected guide RNAs (gRNA1, gRNA2, and gRNA6) on Regnase-1 protein levels. From these data, two guide RNAs, gRNA1 and gRNA6, were selected for further testing.

FIGS. 23A-23B show that human CAR-T Regnase-1-null cells have improved survival ex vivo. FIG. 23A shows survival of human CD4 CAR-T Regnase-1-null cells with the two selected guide RNAs gRNA1 and gRNA6. FIG. 23B shows survival of human CD8 CAR-T Regnase-1-null cells with the two selected guide RNAs gRNA1 and gRNA6.

FIGS. 24A-24B show that human CAR-T Regnase-1-null cells have improved proliferation (FIG. 24A) and reduced apoptosis (FIG. 24B) ex vivo.

FIGS. 25A-25B show that human CD4 Regnase-1-null (FIG. 25A) and CD8 Regnase-1-null (FIG. 25B) CAR-T cells have more memory subsets upon antigen activation ex vivo.

FIGS. 26A-26D show that human CD8 CAR-T Regnase-1-null cells secrete more cytokines ex vivo, specifically IL-2 (FIG. 26A), TNFa (FIG. 26B), IFN-gamma (FIG. 26C), and GrzB (FIG. 26D).

FIGS. 27A-27D show that human CD4 CAR-T Regnase-1-null cells secrete more cytokines ex vivo, specifically IL-2 (FIG. 27A), TNFa (FIG. 27B), IFN-gamma (FIG. 27C), and GrzB (FIG. 27D).

FIG. 28 shows that CD8 Regnase-1-null CAR-T cells can hyperproliferate ex vivo.

FIGS. 29A-29B show that CD8 Regnase-1-null CAR-T na-ve (top panel) and bulk (bottom panel) cells have upregulated mitochondrial activity ex vivo as measured by TMRM (FIG. 29A) and mitotracker (FIG. 29B).

FIG. 30 shows upregulation of genes related to T cell proliferation and mitochondrial activity in Regnase-1-null CAR-T cells ex vivo upon antigen stimulation by GSEA analysis.

FIGS. 31A-31B show that mice treated with Regnase-1-null CAR-T cells in vivo have lower tumor burden as indicated by the luciferase activity of each treatment group (FIG. 31A) and individual recipient (FIG. 31B).

FIGS. 32A-32B show that human Regnase-1-null CAR-T cells have improved cytotoxicity (FIG. 32A) and improved survival (FIG. 32B) ex vivo.

FIGS. 33A-33B show hyperactivation of CD8 Regnase-1-null (FIG. 33A) and CD4 Regnase-1-null (FIG. 33B) CAR-T cells ex vivo.

### DETAILED DESCRIPTION

The present invention generally provides methods for enhancing expansion and/or persistence and/or effector function (e.g., an anti-tumor or an anti-infection function) of T cells. The present invention also provides modified T cells with enhanced expansion and/or persistence and/or effector function (e.g., an anti-tumor or an anti-infection function), as well as pharmaceutical compositions comprising such modified T cells. The present invention further provides methods of using such modified T cells to treat a disease (e.g., cancer or infectious disease) in a subject.

T cells undergo extensive metabolic programing during differentiation and in adaptation to different contexts. T cell longevity and function in cancer immunotherapy have been proposed to be closely correlated with cell metabolic fitness^{13,14}, although the underlying molecular mechanisms are unclear. The present invention is based on an unexpected discovery that Regnase-1 (also known as Zc3h12a or MCPIP1) is a major negative regulator of antitumor responses, whose deficiency results in drastically increased CD8⁺ T cell accumulation in tumors. Data in support of these findings is presented in the Examples section, below. For instance, it was demonstrated that Regnase-1 deficient CD8⁺ T cells are long-lived effector cells with extensive accumulation, better persistence and robust effector function in tumors. Surprisingly, Regnase-1-deficient CD8⁺ T cells show profoundly improved therapeutic efficacy in mouse melanoma and leukemia tumor models. Regnase-1-deficient CD8⁺ T cells are reprogrammed specifically in tumor microenvironment (TME) to acquire naive/memory cell-associated gene signatures for better persistence and survival advantage, but also retain high-level expression of effector molecules such as IFN-y and granzyme B.

The present invention is also based on another unexpected discovery that BATF is a key functional target of Regnase-1 in reprogramming antitumor responses of CD8⁺ T cells. As detailed in the Examples section below, through a secondary in vivo genome-scale CRISPR-Cas9 screening, BATF was identified as the key target of Regnase-1 and a rheostat in shaping antitumor responses. Loss of BATF suppresses the elevated accumulation and mitochondrial fitness of Regnase-1-deficient CD8⁺ T cells. Moreover, genome-scale CRISPR-Cas9 screening also identifies additional genes, such as Ptpn2, Socs1 and Rc3h1, that could be targeted alone or in combination with Regnase-1 and/or Batf to further improve T-cell based therapy.

### Definitions

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Thl), a T helper 2 (Th2) cell, a T helper 17 (Th17) or regulatory T (Treg) cell. The T cell can be a T helper cell (Th; CD4⁺ T cell) CD4⁺ T cell, a cytotoxic T cell (CTL; CD8⁺ T cell), a tumor infiltrating cytotoxic T cell (TIL; CD8⁺ T cell), CD4⁺ CD8⁺ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺and CD8⁻ cells. The TCR on NKT cells is unique in that it recognizes glycolipid antigens presented by the MEW I-like molecule CD Id. NKT cells can have either protective or deleterious effects due to their abilities to produce cytokines that promote either inflammation or immune tolerance. Also included are “gamma-delta T cells (γϵ T cells),” which refer to a specialized population that to a small subset of T cells possessing a distinct TCR on their surface, and unlike the majority of T cells in which the TCR is composed of two glycoprotein chains designated α- and β TCR chains, the TCR in γϵ T cells is made up of a γ-chain and a δ-chain. γϵ T cells can play a role in immunosurveillance and immunoregulation, and were found to be an important source of IL-17 and to induce robust CDS⁺cytotoxic T cell response. Also included are “regulatory T cells” or “Tregs” refers to T cells that suppress an abnormal or excessive immune response and play a role in immune tolerance. Tregs cells are typically transcription factor Foxp3-positive CD4⁺ T cells and can also include transcription factor Foxp3-negative regulatory T cells that are IL-10-producing CD4⁺ T cells.

The terms “natural killer cell” and “NK cell” are used interchangeable and used synonymously herein. As used herein, NK cell refers to a differentiated lymphocyte with a CD 16⁺ CD56⁺and/or CD57⁺ TCR- phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The term “chimeric antigen receptor” or “CAR” as used herein is defined as a cell-surface receptor comprising an extracellular target-binding domain, a transmembrane domain and a cytoplasmic domain, comprisilng a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present invention are intended primarily for use with lymphocyte such as T cells and natural killer (NK) cells.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′)2, and Fv fragments; polypeptides derived from T Cell receptors, such as, for example, TCR variable domains; secreted factors (e.g., cytokines, growth factors) that can be artificially fused to signaling domains; and any ligand or receptor fragment (e.g., CD27, NKG2D) that binds to the antigen of interest. Combinatorial libraries could also be used to identify peptides binding with high affinity to the therapeutic target.

Terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab') fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgMl, IgM2, IgA1 and IgA2) or subclass.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” or “genetically engineered” refers to the addition of extra genetic material in the form of DNA or RNA into a cell.

As used herein, the term “derivative” in the context of proteins or polypeptides (e.g., CAR constructs or domains thereof) refer to: (a) a polypeptide that has at least 40%, 45%, 50%, 55%, 60%, 65%, 70%, 75%, 80%, 85%, 90%, 95%, 98%, 99%, or 99.5% sequence identity to the polypeptide it is a derivative of; (b) a polypeptide encoded by a nucleotide sequence that has at least 40%, 45%, 50%, 55%, 60%, 65%, 70%, 75%, 80%, 85%, 90%, 95%, 98%, 99%, or 99.5% sequence identity to a nucleotide sequence encoding the polypeptide it is a derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative of (d) a polypeptide encoded by nucleic acids can hybridize under high, moderate or typical stringency hybridization conditions to nucleic acids encoding the polypeptide it is a derivative of; (e) a polypeptide encoded by a nucleotide sequence that can hybridize under high, moderate or typical stringency hybridization conditions to a nucleotide sequence encoding a fragment of the polypeptide, it is a derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative of.

The term “functional fragment” or “functional derivative” as used herein refers to a fragment or derivative of the polypeptide or protein, or a polynucleotide encoding the polypeptide or protein, that retains at least one function of the full-length polypeptide or protein, or the polypeptide or protein it is a derivative of A functional fragment may comprise an amino acid sequence of at least 5 contiguous amino acid residues, at least 6 contiguous amino acid residues, at least 7 contiguous amino acid residues, at least 8 contiguous amino acid residues, at least 9 contiguous amino acid residues, at least 10 contiguous amino acid residues, at least 11 contiguous amino acid residues, at least 12 contiguous amino acid residues, at least 13 contiguous amino acid residues, at least 14 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of the full-length polypeptide or protein. The functional fragment or functional derivative of a polypeptide or protein may retain one, two, three, four, five, or more functions of the full-length polypeptide or protein, or the polypeptide or protein it is a derivative of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The terms “enhance” or “promote” or “increase” or “expand” or “improve” refer generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

The terms “decrease” or “lower,” or “lessen,” or “reduce,” or “abate” refer generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington' s Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, N J; Bonifacino et al. eds. (2005) Current Protocols in Cell Biology. John Wiley and Sons, Inc.: Hoboken, N J; Coligan et al. eds. (2005) Current Protocols in Immunology, John Wiley and Sons, Inc.: Hoboken, N J; Coico et al. eds. (2005) Current Protocols in Microbiology, John Wiley and Sons, Inc.: Hoboken, N J; Coligan et al. eds. (2005) Current Protocols in Protein Science, John Wiley and Sons, Inc.: Hoboken, N J; and Enna et al. eds. (2005) Current Protocols in Pharmacology, John Wiley and Sons, Inc.: Hoboken, N J. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Methods of Enhancing T Cell Function

In one aspect, the present disclosure provides a method of enhancing expansion and/or persistence and/or an anti-tumor or an anti-infection function of a T cell. The method includes modifying a Regnase-1 gene or gene product in the T cell such that the expression and/or function of Regnase-1 in the T cell is reduced or eliminated.

The terms “expand” or “expansion” when used in relation to a T cell refer to the ability of the T cell to undergo cellular proliferation (i.e., to increase the number of cells). The terms used herein encompass both in vivo and in vitro T cell expansion.

The terms “persist” or “persistence” when used in relation to a T cell refer to the ability of the T cell (and/or its progenies) to be maintained in a recipient (e.g., a subject) for a period of time. The terms used herein encompass both in vivo and in vitro T cell persistence.

The term “anti-tumor function” as used herein refers to the ability of a T cell to inhibit tumor growth and/or to kill the tumor cells (cancer cells).

The term “anti-infection function” as used herein refers to the ability of a T cell to inhibit the growth of a pathogen or a population of pathogens and/or kill a pathogen or a population of pathogens. A pathogen may be a virus, a bacterium, a fungus, a parasite, or a prion, or the like.

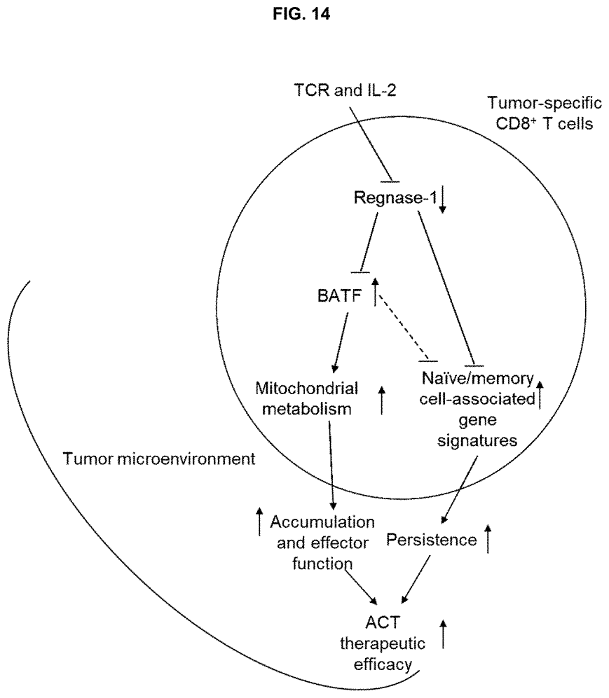

Regnase-1, also known as Zc3h12a or MCPIP1, is an RNase that destabilizes a set of mRNAs, through cleavage of their 3′ untranslated regions (UTRs). The Regnase-1 gene has NCBI gene IDs of 80149 (human) and 230738 (mouse). As described in the Examples section below, Regnase-1 was identified as a major regulator of T cell effector responses, whose deficiency can cause reprogramming of T cells (specifically in the TME), resulting in markedly improved therapeutic efficacy. While not wishing to be bound by theory, Regnase-1 may function after initial T cell activation^{15,16} to enable precise temporal and spatial control of effector T cell responses. Further, Regnase-1 may restrain mitochondrial oxidative metabolism to limit effector T cell responses in tumor immunity, and function through a gene target BATF (FIG. 14).

T cells that may be used in the present disclosure include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺ CD8⁺ T cell, or any other subset of T cells. Other illustrative populations of T cells suitable for use in particular embodiments include naive T cells memory T cells, and NKT cells.

In some embodiments, the T cell a CD8⁺ αβ T cell receptor (TCR) T cell, a CD4⁺ αβ TCR T cell, a regulatory T cell (Treg), a natural killer T (NKT) cell, or a γϵ T cell. In a specific embodiment, the T cell is a CD8⁺ αβ TCR T cell. In another specific embodiment, the T cell is a CD4⁺ αβ TCR T cell. In a specific embodiment, the T cell is a regulatory T cell (Treg). The T cell may have the ability to target a tumor antigen or an infectious antigen.

T cells may be further engineered to express a T cell receptor or a chimeric antigen receptor (CAR). The T cell receptor or CAR may have an antigen-binding moiety that is capable of targeting a tumor antigen or an infectious antigen.

Non-limiting examples of tumor antigens that may be targeted by the modified T cell described herein include human epidermal growth factor receptor 2 (HER2), interleukin-13 receptor subunit alpha-2 (IL-13Ra2), ephrin type-A receptor 2 (EphA2), A kinase anchor protein 4 (AKAP-4), adrenoceptor beta 3 (ADRB3), anaplastic lymphoma kinase (ALK), immunoglobulin lambda- like polypeptide 1 (IGLL1), androgen receptor, angiopoietin-binding cell surface receptor 2 (Tie 2), B7H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLEC12A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (T1VIPRSS2) ETS fusion gene), ETS translocation-variant gene 6, located on chromosome 12p (ETV6-AML), Fc fragment of IgA receptor (FCAR), Fc receptor-like 5 (FCRLS), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRCSD), ganglioside GD3, ganglioside GM3, glycoceramide (GloboH), Glypican-3 (GPC3), Hepatitis A virus cellular receptor 1 (HAVCR1), hexasaccharide portion of globoH, high molecular weight-melanoma-associated antigen (HMWMAA), human Telomerase reverse transcriptase (hTERT), interleukin 11 receptor alpha (IL-11Ra), KIT (CD117), leukocyte-associated immunoglobulin-like receptor 1 (LAIR1), leukocyte immunoglobulin-like receptor subfamily A member 2 (LILRA2), Lewis(Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAXS), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEM1/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WT1), and X Antigen Family, Member 1A (XAGE1), or a fragment or variant thereof.

Additional antigens that may be targeted by the modified T cell described herein include, but are not limited to, carbonic anhydrase EX, alpha-fetoprotein, A3, antigen specific for A33 antibody, Ba 733, BrE3-antigen, CA125, CD1, CD1a, CD3, CDS, CD15, CD16, CD19, CD20, CD21, CD22, CD23, CD25, CD30, CD33, CD38, CD45, CD74, CD79a, CD80, CD138, colon-specific antigen-p (CSAp), CEA (CEACAMS), CEACAM6, CSAp, EGFR, EGP-I, EGP-2, Ep-CAM, EphAl, EphA3, EphA4, EphA5, EphA6, EphA7, EphA8, EphAl 0, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

An infectious antigen may be a viral antigen, a bacterial antigen, a fungal antigen, a parasite antigen, or a prion antigen, or the like. Infectious antigens include the intact microorganism (e.g., virus, bacterium, fungus) as well as natural isolates and fragments or derivatives thereof and also synthetic or recombinant compounds which are identical to or similar to natural microorganism antigens and induce an immune response specific for that microorganism (e.g., virus, bacterium, fungus). A compound is similar to a natural microorganism antigen if it induces an immune response (humoral and/or cellular) to a natural microorganism antigen. Such antigens are used routinely in the art and are well known to the skilled artisan.

An infectious antigen may be an infectious virus or derived from an infectious virus. Non-limiting examples of infectious viruses that have been found in humans include but are not limited to: Adenoviridae (most adenoviruses); Arena viridae (hemorrhagic fever viruses); Birnaviridae; Bungaviridae (e.g., Hantaan viruses, bunga viruses, phleboviruses and Nairo viruses); Calciviridae (e.g., strains that cause gastroenteritis); Coronoviridae (e.g., coronaviruses); Filoviridae (e.g., ebola viruses); Flaviridae (e.g., dengue viruses, encephalitis viruses, yellow fever viruses); Hepadnaviridae (Hepatitis B virus); Herpesviridae (herpes simplex virus (HSV) 1 and 2, varicella zoster virus, cytomegalovirus (CMV), herpes virus); Iridoviridae (e.g., African swine fever virus); Norwalk and related viruses, and astroviruses.; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvovirida (parvoviruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviurses and rotaviruses); Retroviridae (e.g., human immunodeficiency viruses, such as HIV-1 (also referred to as HTLV-III, LAV or HTLV-III/LAV, or HIV-III); and other isolates, such as HIV-LP); Rhabdoviradae (e.g., vesicular stomatitis viruses, rabies viruses); Togaviridae (e.g., equine encephalitis viruses, rubella viruses); and unclassified viruses (e.g., the etiological agents of Spongiform encephalopathies, the agent of delta hepatitis, the agents of non-A, non-B hepatitis (class 1=internally transmitted; class 2=parenterally transmitted (i.e. Hepatitis C)).

An infectious antigen may be an infectious bacterium or derived from an infectious bacterium. Both gram negative and gram positive bacteria can serve as antigens in vertebrate animals. Such gram positive bacteria include, but are not limited to, Pasteurella species, Staphylococci species and Streptococcus species. Grain negative bacteria include, but are not limited to, Escherichia coli, Pseudomonas species, and Salmonella species. Non-limiting examples of infectious bacteria include but are not limited to: Actinomyces israelli, Bacillus antracis, Bacteroides sp., Borelia burgdorferi, Chlamydia., Clostridium perfringens, Clostridium tetani, Corynebacterium diphtherias, Corynebacterium sp., Enterobacter aerogenes, Enterococcus sp., Erysipelothrix rhusiopathiae, Fusobacterium nucleatum, Haemophilus influenzae, Helicobacter pyloris, Klebsiella pneumoniae, Legionella pneumophilia, Leptospira, Listeria monocytogenes, Mycobacteria sps. (e.g., M tuberculosis, M avium, M gordonae, M intracellulare, M kansaii), Neisseria gonorrhoeae, Neisseria meningitidis, Pasturella multocida, pathogenic Campylobacter sp., Rickettsia, Staphylococcus aureus, Streptobacillus monihformis, Streptococcus (anaerobic sps.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidium, and Treponema pertenue.

An infectious antigen may be or derived from other infectious microorganisms. Non-limiting examples of infectious fungi include Cryptococcus neoformans, Histoplasma capsulatuin, Coccidioides immitis, Blastomyces dernatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma. Other medically relevant microorganisms have been descried extensively in the literature, e.g., see C. G. A. Thomas, “Medical Microbiology”, Bailliere Tindall, Great Britain 1983, which is hereby incorporated by reference in its entirety.

Other non-limiting examples of infectious antigens include viral antigens such as HIV antigens (e.g., gp120, gp160, p18, Tat, Gag, Pol, Env, Nef), glycoprotein from Herpesvirus, and surface antigen and core antigen from Hepatitis B virus; bacterial antigens such as OspA, OspB and OspC antigens from Borrelia sp; fungal and parasite antigens such as MP65 from Candida albicans and CS protein from Plasmodium sp.

In some embodiments, modifying the Regnase-1 gene and/or gene product in the T cell improves in vivo accumulation of the T cell. In some embodiments, the in vivo accumulation of the T cell is improved more than at least about 10-fold as compared an unmodified T cell measured at day 7 after the Regnase-1 modification. In some embodiments, the in vivo accumulation of the T cell is improved more than at least about 10-fold, about 30-fold, about 50-fold, about 70-fold, about 90-fold, about 100-fold, about 110-fold, about 120-fold, about 130-fold, about 140-fold, about 150-fold, about 160-fold, about 180-fold, about 200-fold, about 230-fold, about 250-fold or more as compared an unmodified T cell measured at day 7 after the Regnase-1 modification.

In some embodiments, the in vivo accumulation of the T cell is improved more than at least about 100-fold as compared an unmodified T cell measured at day 14 after the Regnase-1 modification. In some embodiments, the in vivo accumulation of the T cell is improved more than at least about 100-fold, about 200-fold, about 300-fold, about 400-fold, about 500-fold, about 550-fold, about 600-fold, about 650-fold, about 670-fold, about 690-fold, about 700-fold, about 710-fold, about 720-fold, about 730-fold, about 740-fold, about 750-fold, about 760-fold, about 770-fold, about 780-fold, about 790-fold, about 800-fold, about 900-fold, about 1000-fold or more as compared an unmodified T cell measured at day 14 after the Regnase-1 modification.

In some embodiments, the in vivo accumulation of the T cell is improved more than at least about 1000-fold as compared an unmodified T cell measured at day 21 after the Regnase-1 modification. In some embodiments, the in vivo accumulation of the T cell is improved more than at least about 1000-fold, about 1100-fold, about 1200-fold, about 1300-fold, about 1400-fold, about 1500-fold, about 1600-fold, about 1700-fold, about 1800-fold, about 1900-fold, about 2000-fold, about 2100-fold, about 2200-fold, about 2300-fold, about 2350-fold, about 2400-fold, about 2500-fold, about 2550-fold, about 2600-fold, about 2700-fold, about 2800-fold, about 2900-fold, about 3000-fold, or more as compared an unmodified T cell measured at day 14 after the Regnase-1 modification.

In addition to Regnase-1, additional gene(s) or gene product(s) in the T cell may be modified alone or in combination with Regnase-1 to enhance expansion and/or persistence and/or an anti-tumor or an anti-infection function of a T cell. Additional genes or gene products that may be modified can be selected from Ptpn2, Socs1, Agps, Rc3h1, and Rcor1. Other suitable genes or gene products that may be modified include Ireb2, Vtila, or Pex13. Additional suitable genes or gene products that may be modified include those listed in Table 1. Modifying one or more of such genes or gene products in addition to Regnase-1 may have synergetic or additive effects in enhancing expansion and/or persistence and/or an anti-tumor or an anti-infection function of a T cell.

Ptpn2 has NCBI gene IDs of 5771 (human) and 19255 (mouse). Socs1 has NCBI gene IDs of 8651 (human) and 12703 (mouse). Agps has NCBI gene IDs of 8540 (human) and 228061 (mouse). Rc3h1 (Roquin-1) has NCBI gene IDs of 149041 (human) and 381305 (mouse). Rcor1 has NCBI gene IDs of 23186 (human) and 217864 (mouse). Ireb2 has NCBI gene IDs of 3658 (human) and 64602 (mouse). Vtila has NCBI gene IDs of 143187 (human) and 53611 (mouse). Pex13 has NCBI gene IDs of 5194 (human) and 72129 (mouse).

In some embodiments, the Regnase-1 gene and/or any additional gene(s) (e.g., Ptpn2, Socs1, Agps, Rc3h1, Rcor1, Ireb2, Vtila, or Pex13) in the T cell are modified with a site-specific nuclease. The term “site-specific nuclease” as used herein refers to a nuclease capable of specifically recognizing and cleaving a nucleic acid (DNA or RNA) sequence. Suitable site-specific nucleases for use in the present invention include, but are not limited to, an RNA-guided endonuclease (e.g., CRISPR-associated (Cas) proteins), a zinc finger nuclease, a TALEN nuclease, or a mega-TALEN nuclease.

Site-specific nucleases may create double-strand breaks (DSBs) or single-strand breaks (i.e., nicks) in a genomic DNA of a cell. Although not wishing to be bound by theory, these breaks are typically repaired by the cell using one of two mechanisms: non-homologous end joining (NHEJ) and homology-directed repair (HDR). In NHEJ, the double-strand breaks are repaired by direct ligation of the break ends to one another. As a result, no new nucleic acid material is inserted into the site, although a few bases may be lost or added, resulting in a small insertions and deletion (indel). In HDR, a donor polynucleotide with homology to the cleaved target DNA sequence is used as a template to repair the cleaved target DNA sequence, resulting in the transfer of genetic information from the donor polynucleotide to the target DNA. As such, new nucleic acid material may be inserted or copied into the cleavage site. In some cases, an exogenous donor polynucleotide can be provided to the cell. The modifications of the target DNA due to NHEJ and/or HDR may lead to, for example, gene correction, gene replacement, gene tagging, transgene insertion, nucleotide deletion, gene disruption, gene mutation, sequence replacement, etc. Accordingly, cleavage of DNA by a site-directed nuclease may be used to delete nucleic acid material from a target DNA sequence by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material (e.g., a transgene) into a locus of choice in the target DNA.

In some embodiments, the site-specific nuclease is an RNA-guided endonuclease. In particular, a group of RNA-guided endonucleases known as CRISPR-associated (Cas) proteins may be employed to genetically modify the T cell. A Cas protein may form an RNA-protein complex (referred to as RNP) with a guide RNA (gRNA) and is capable of cleaving a target site bearing sequence complementarity to a short sequence (typically about 20-40nt) in the gRNA.

Examples of Cas proteins useful in the methods of the present disclosure include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Cpf1, Csy 1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb 1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In some embodiments, the Cas protein used in the methods described herein is a Cas9 protein. The Cas9 protein may be from S. pyogenes, Streptococcus thermophilus, Neisseria meningitidis, F. novicida, S. mutans or Treponema denticola.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99%, 99.5% or more sequence identity to the wild type or modified Cas protein or a portion thereof, wherein the active variants retain the ability to cut at a desired cleavage site and hence retain nick-inducing or double-strand-break-inducing activity.

A “guide RNA” or “gRNA” is an RNA molecule that binds to a Cas protein (e.g., Cas9 protein), or functional fragment or derivative thereof, and targets the Cas protein to a specific location within a target DNA. In some embodiments, the guide RNA is a single guide RNA (sgRNA). For Cas9, for example, a single-guide RNA can comprise a crRNA fused to a tracrRNA (e.g., via a linker). In some embodiments, the sgRNA is designed to target a locus within or near the Regnase-1 gene. In some embodiments, the sgRNA is designed to target a locus within or near the Ptpn2 gene. In some embodiments, the sgRNA is designed to target a locus within or near the Socs1 gene. In some embodiments, the sgRNA is designed to target a locus within or near the Agps gene. In some embodiments, the sgRNA is designed to target a locus within or near the Rc3h1 gene. In some embodiments, the sgRNA is designed to target a locus within or near the Rcor 1 gene. In some embodiments, the sgRNA is designed to target a locus within or near the Ireb2 gene. In some embodiments, the sgRNA is designed to target a locus within or near the Vti1a gene. In some embodiments, the sgRNA is designed to target a locus within or near the Pex13 gene.

Exemplary sgRNAs useful for modifying a target gene described in the present disclosure include those that comprise a nucleotide sequence set forth in any one of SEQ ID NOs: 1, 2, and 5-9. For example, a sgRNA targeting the Regnase-1 gene may comprise a nucleotide sequence of SEQ ID NO: 1 or SEQ ID NO: 2, or a variant having at least 50%, at least 55%, at least 60%, at least 65%, at least 70%, at least 75%, at least 80%, at least 85%, at least 90%, or at least 95% sequence identity with SEQ ID NO: 1 or SEQ ID NO: 2. For example, a sgRNA targeting the Ptpn2 gene may comprise a nucleotide sequence of SEQ ID NO: 5 or SEQ ID NO: 6, or a variant having at least 50%, at least 55%, at least 60%, at least 65%, at least 70%, at least 75%, at least 80%, at least 85%, at least 90%, or at least 95% sequence identity with SEQ ID NO: 5 or SEQ ID NO: 6. For example, a sgRNA targeting the Socs1 gene may comprise a nucleotide sequence of SEQ ID NO: 7 or SEQ ID NO: 8, or a variant having at least 50%, at least 55%, at least 60%, at least 65%, at least 70%, at least 75%, at least 80%, at least 85%, at least 90%, or at least 95% sequence identity with SEQ ID NO: 7 or SEQ ID NO: 8. For example, a sgRNA targeting the Agps gene may comprise a nucleotide sequence of SEQ ID NO: 9, or a variant having at least at least 50%, at least 55%, at least 60%, at least 65%, at least 70%, at least 75%, at least 80%, at least 85%, at least 90%, or at least 95% sequence identity with SEQ ID NO: 9. For example, a sgRNA targeting the Rc3h1 gene may comprise a nucleotide sequence of SEQ ID NO: 42, or a variant having at least at least 50%, at least 55%, at least 60%, at least 65%, at least 70%, at least 75%, at least 80%, at least 85%, at least 90%, or at least 95% sequence identity with SEQ ID NO: 42.

Further exemplary sgRNAs targeting the Regnase-1 gene include those that comprise a nucleotide sequence set forth in any one of SEQ ID NOs: 29-34 and 36-41. For example, an sgRNA targeting the Regnase-1 gene may comprise a nucleotide sequence of any one of SEQ ID NOs: 29-34 and 36-41, or a variant having at least 50%, at least 55%, at least 60%, at least 65%, at least 70%, at least 75%, at least 80%, at least 85%, at least 90%, or at least 95% sequence identity with SEQ ID NOs: 29-34 and 36-41.

In one embodiment, the sgRNA targeting the Regnase-1 gene comprises a nucleotide sequence of SEQ ID NO: 29. In another embodiment, the sgRNA targeting the Regnase-1 gene comprises a nucleotide sequence of SEQ ID NO: 34.

In one embodiment, the sgRNA targeting the Regnase-1 gene comprises a nucleotide sequence of SEQ ID NO: 36. In another embodiment, the sgRNA targeting the Regnase-1 gene comprises a nucleotide sequence of SEQ ID NO: 41.

In alternative embodiments, the site-specific nuclease used in the methods described herein is a zinc finger nuclease, a transcription activator-like effector nuclease (TALEN), a mega-TALEN nuclease, and/or a restriction endonuclease.

The site-specific nuclease used in the methods described herein may include a zinc finger nuclease (ZFN). Zinc finger nucleases (ZFNs) are a class of engineered DNA-binding proteins that assist targeted editing of the genome by creating double-strand breaks in DNA at targeted locations. ZFNs typically comprise two functional domains: a) a DNA-binding domain comprising a chain of two-finger modules (each recognizing a unique hexamer (6 bp) sequence of DNA-two-finger modules are stitched together to form a Zinc Finger Protein, each with specificity of about 24 bp or more) and b) a DNA-cleaving domain comprising the nuclease domain of Fok I. When the DNA-binding and -cleaving domains are fused together, a ZFN can act like a highly-specific pair of “genomic scissors”.

The site-specific nuclease used in the methods described herein may include a transcription activator-like effector nuclease (TALEN). Transcription activator-like effector nucleases (TALEN) are a class of sequence-specific nucleases that can be used to make double-strand breaks at specific target sequences in the genome of a prokaryotic or eukaryotic organism. They typically comprise a TAL effector DNA-binding domain fused to a DNA cleavage domain (a nuclease which cuts DNA strands). TAL effector nucleases can be created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. doi: 10.1093/nar/gkq704; and Miller et al. (2011) Nature Biotechnology 29:143-148; all of which are herein incorporated by reference in their entirety and for all purposes.

In some embodiments, the method involves silencing a Regnase-1 mRNA with an RNA interference (RNAi) molecule or an antisense oligonucleotide. RNA interference (RNAi) refers to the process of sequence-specific post-transcriptional gene silencing in animals mediated by small interfering RNAs (siRNAs) (Fire et al., 1998, Nature, 391, 806; Hamilton et al., 1999, Science, 286, 950-951). Any small nucleic acid molecules capable of mediating RNAi, such as a short interfering nucleic acid (siNA), a small interfering RNA (siRNA), a double-stranded RNA (dsRNA), a micro-RNA (miRNA), and a short hairpin RNA (shRNA), may be used to inhibit the expression of the Regnase-1 gene. An antisense oligonucleotide (ASO) is a short nucleotide sequence that can hybridize or bind (e.g., by Watson-Crick base pairing) in a complementary fashion to its target sequence.

In some embodiments, the RNAi molecule is a small interfering RNA (siRNA) or a small hairpin RNA (shRNA). siRNAs, also known as short interfering RNA or silencing RNA, are a class of double-stranded RNA molecules, 20-25 base pairs in length, and operating within the RNA interference (RNAi) pathway. shRNAs or short hairpin RNAs are a group of artificial RNA molecules with a tight hairpin turn that can be used to silence target gene expression via RNA interference (RNAi).

In some embodiments, the methods also include inhibiting a Regnase-1 protein with one or more of a small molecule inhibitor, a peptide, an antibody or antibody fragment, and an aptamer.

In some embodiments, Regnase-1 inhibitors may include a Zc3h12a gene inhibitor and a Zc3h12a protein inhibitor as those described in U.S. Pat. No. 8,894,996, which is incorporated herein by reference in its entirety.

In another aspect, the present disclosure provides a method of enhancing expansion and/or persistence and/or an anti-tumor or an anti-infection function of a T cell, comprising increasing the expression of Batf gene and/or enhancing the function of BATF protein in the T cell.

BATF, also known as basic leucine zipper transcription factor, ATF-like, is a nuclear basic leucine zipper protein that belongs to the AP-1/ATF superfamily of transcription factors. The Batf gene has NCBI gene IDs of 10538 (human) and 53314 (mouse). As described in the Examples section below, BATF was identified as a key target of Regnase-1. BATF may serve as a limiting factor for programming effective antitumor responses, in part through shaping mitochondrial metabolism.

In some embodiments, the method comprises introducing into the T cell a polynucleotide encoding a BATF protein, or functional fragment or derivative thereof.

As a non-limiting example, the BATF protein encoded by the polynucleotide comprises the amino acid sequence of MPHS SD S SD S SF SRSPPPGKQD S SDDVRRVQRREKNRIAAQKSRQRQTQKADTLHLE SEDLEKQNAALRKEIKQLTEELKYFTSVLNSHEPLC SVLAASTP SPPEVVYSAHAFHQ PHVSSPRFQP (Homo sapiens; UniProtKB-Q16520; SEQ ID NO: 25), or an amino acid sequence having at least 70%, 75%, 80%, 85%, 90%, 91%, 92%, 93%, 94%, 95%, 96%, 97%, 98%, 99%, 99.5% or more sequence identity to SEQ ID NO: 25. As another non-limiting example, the BATF protein encoded by the polynucleotide comprises the amino acid sequence of MPHS SD S SD S SF SRSPPPGKQD S SDDVRKVQRREKNRIAAQKSRQRQTQKADTLHLE SEDLEKQNAALRKEIKQLTEELKYFTSVLSSHEPLCSVLASGTPSPPEVVYSAHAFHQ PHISSPRFQP (Mus musculus; UniProtKB-035284; SEQ ID NO: 26), or an amino acid sequence having at least 70%, 75%, 80%, 85%, 90%, 91%, 92%, 93%, 94%, 95%, 96%, 97%, 98%, 99%, 99.5% or more sequence identity to SEQ ID NO: 26.

As a non-limiting example, the polynucleotide encoding a BATF protein comprises the nucleotide sequence of aaagcgagcgacatgtecctttggggagcagtccctctgcaccccagagtgaggaggacgcaggggtcagaggtggctacagggc aggcagaggaggcacctgtagggggtggtgggctggtggcccaggagaagtcaggaagggagcccagctggtgacaagagagc ccagaggtgcctggggctgagtgtgagagcccggaagatttcagccatgcctcacagctccgacagcagtgactccagatcagcc gctctectccccctggcaaacaggactcatctgatgatgtgagaagagttcagaggagggagaaaaatcgtattgccgcccagaaga gccgacagaggcagacacagaaggccgacaccctgcacctggagagcgaagacctggagaaacagaacgcggctctacgcaag gagatcaagcagctcacagaggaactgaagtacttcacgteggtgctgaacagccacgagcccctgtgctcggtgctggccgccag cacgccctcgccccccgaggtggtgtacagcgcccacgcattccaccaacctcatgtcagctccccgcgcttccagccctgagcttcc gatgeggggagagcagagcctegggaggggcacacagactgtggcagagctgcgcccatcccgcagaggccectgtccacctgg agacccggagacagaggcctggacaaggagtgaacacgggaactgtcacgactggaagggcgtgaggcctcccagcagtgccg cagcgtttcgaggggcgtgtgctggaccccaccactgtgggttgcaggcccaatgcagaagagtattaagaaagatgctcaagtecc atggcacagagcaaggcgggcagggaacggttatttttctaaataaatgctttaaaagaaa (Homo sapiens; NCBI gene ID of 10538; SEQ ID NO: 27), or a nucleotide sequence having at least 70%, 75%, 80%, 85%, 90%, 91%, 92%, 93%, 94%, 95%, 96%, 97%, 98%, 99%, 99.5% or more sequence identity to SEQ ID NO: 27. As another non-limiting example, the polynucleotide encoding a BATF protein comprises the nucleotide sequence of gcagtecctctgcacccgagagagaggaggacgcaggggtctgtcagaggttgctgttgggcaagcaggggaggtacctgtggaa ggtggtgtgctggtggcccectagcagtcaagaaggggagccagctagtgagaagatcgcccagaggcatctgggacggtgtggg agagcccggaagattagaaccatgcctcacagctccgacagcagtgactccagatcagccgctctcctccccctggcaaacaggac tcatctgatgatgtgaggaaagttcagaggagagagaagaatcgcatcgctgcccagaagagccgacagagacagacacagaaag ccgacaccatcacctggagagtgaggacctggagaaacagaacgcagctctccgcaaagagatcaaacagctcaccgaggagct caagtacttcacatcagtgctgagcagccacgagccectgtgctccgtgctggccagtggcaccccctcgccccccgaggtggtata cagtgcccatgccttccaccagcctcacatcagctcgccacgcttccagccctgaccttctggacaagaagggcgatgctactcccgt gatccatggaggggcatgtaaactgaggccgggctgccctcatacctctacccagaggcccagtggcagaggcctggacaagtatt gaacacaagaactgtagtggtcagagggacttaaggcctcccagggaagtatagtcaatgtactggactctcccagggaagtcgagc caatgtactggacccaaaaaatgacaagtcaaccctggactgtcatgaatgatgcccaaaatacacagcacagagggaggagggca gggggtggatagttttctaaataaatattttctaaaaaacca (Mus musculus; NCBI gene ID of 53314; SEQ ID NO: 28), or a nucleotide sequence having at least 70%, 75%, 80%, 85%, 90%, 91%, 92%, 93%, 94%, 95%, 96%, 97%, 98%, 99%, 99.5% or more sequence identity to SEQ ID NO: 28.

In certain embodiments, the polynucleotide encoding a BATF protein, or functional fragment or derivative thereof, is introduced into the T cell in a recombinant vector. The recombinant vector may be a viral vector or non-viral vector, such as those described herein.

In certain embodiments, increasing the expression of Batf gene and/or enhancing the function of BATF protein in the T cell is achieved by administering to the T cell an agent (e.g., a small molecule or an antibody) that upregulates Batf gene expression and/or directly enhances or activates the BATF protein function.

In certain embodiments, in addition to increasing the expression of Batf gene and/or enhancing the function of BATF protein in the T cell, the method further comprises modifying one or more additional genes or gene products in the T cell such that the expression and/or function of the additional gene(s) or gene product(s) in the T cell is reduced or eliminated, wherein the additional gene(s) or gene product(s) are selected from Regnase-1 (REGNASE-1, Zc3h12a, MCPIP1), Ptpn2, Socs1, Agps, Rc3h1, and Rcor1. In some embodiments, the additional gene(s) or gene product(s) is Regnase-1 (REGNASE-1, Zc3h12a, MCPIP1).

In another aspect, the present disclosure provides a method of enhancing expansion and/or persistence and/or an anti-tumor or an anti-infection function of a T cell, comprising modifying a Regnase-1 (REGNASE-1, Zc3h12a, MCPIP1) gene or gene product in the T cell such that the expression and/or function of Regnase-1 in the T cell is reduced or eliminated and increasing the expression of Batf gene and/or enhancing the function of BATF protein in the T cell. In certain embodiments, the method further comprises modifying one or more additional genes or gene products in the T cell such that the expression and/or function of said additional gene(s) or gene product(s) in said T cell is reduced or eliminated, wherein said additional gene(s) or gene product(s) are selected from Ptpn2, Socs1, Agps, Rc3h1, and Rcor1.

In another aspect, the present disclosure provides a method of improving mitochondrial biogenesis and/or function in a T cell comprising modifying a Regnase-1 (REGNASE-1, Zc3h12a, MCPIP1) gene or gene product in the T cell such that the expression and/or function of Regnase-1 in the T cell is reduced or eliminated and/or increasing the expression of Batf gene and/or enhancing the function of BATF protein in the T cell. In certain embodiments, the method further comprises modifying one or more additional genes or gene products in the T cell such that the expression and/or function of said additional gene(s) or gene product(s) in said T cell is reduced or eliminated, wherein said additional gene(s) or gene product(s) are selected from Ptpn2, Socs1, Agps, Rc3h1, and Rcor1.

As used herein, the term “mitochondrial biogenesis” refers to the process by which cells increase mitochondrial mass. As used herein, the term “mitochondrial function” refers to any mitochondria-dependent cellular metabolism.

Effects of improved mitochondrial function and/or mitochondrial biogenesis may include without limitation observations that the method of this invention improves T accumulation and function in tumors, helps promote endurance, helps promote recovery after exercise, helps reduce muscle fatigue, helps reduce muscle soreness, complements the immediate short term effect of caffeine with a sustained effect on energy generation, helps promote energy generation from fat, helps lower plasma lactate during exercise, helps maintain muscle force in conditions of oxidative stress, helps protect against exercise-induced oxidative stress, helps the body to come up with more energy in a natural and sustained way, helps the body to find more energy without getting too much caffeine, gives long-lasting energy to sustain through busy schedule, helps boost body's own energy production in a natural sustained way, helps maintain more even energy levels throughout the day, helps the body to adapt to exercise, helps prepare the body for exercise goals, helps revamp shape, facilitates the restart of exercise program, increased muscle work capacity, improved aerobic capacity, enhanced physical performance, enhanced exercise performance, improved running endurance, improved running distance and/or improved running time, or stimulate energy formation from nutrients.

Isolation/Enrichment of T cells

The T cells may be autologous/autogeneic (“self”) or non-autologous (“non-self,” e.g., allogeneic, syngeneic or xenogeneic). In some embodiments, the T cells are obtained from a mammalian subject. In other embodiments, the T cells are obtained from a primate subject. In some embodiments, the T cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In some embodiments, lymphocytes can be obtained from blood collected from a subject using techniques generally known to the skilled person, such as sedimentation, e.g., FICOLL™ separation.

In some embodiments, the T cell is derived from a blood, marrow, tissue, or tumor sample.

In some embodiments, cells from the circulating blood of a subject are obtained by apheresis. An apheresis device typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In some embodiments, T cells can be isolated from peripheral blood mononuclear cells (PBMCs) by lysing the red blood cells and depleting the monocytes. As an example, the cells can be sorted by centrifugation through a PERCOLL™ gradient. In some embodiments, after isolation of PBMC, both cytotoxic and helper T lymphocytes can be sorted into naive, memory, and effector T cell subpopulations either before or after activation, expansion, and/or genetic modification.

In some embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In some embodiments, the T lymphocytes for use in the compositions of the invention do not express or do not substantially express one or more of the following markers: CD57, CD244, CD160, PD-1, CTLA4, TIM3, and LAG3.

Stimulation/Activation of T cells

In order to reach sufficient therapeutic doses of T cell compositions, T cells are often subjected to one or more rounds of stimulation/activation. In some embodiments, a method of producing T cells for administration to a subject comprises stimulating the T cells to become activated in the presence of one or more stimulatory signals or agents (e.g., compound, small molecule, e.g., small organic molecule, nucleic acid, polypeptide, or a fragment, isoform, variant, analog, or derivative thereof). In some embodiments, a method of producing T cells for administration to a subject comprises stimulating the T cells to become activated and to proliferate in the presence of one or more stimulatory signals or agents.

T cells can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In some embodiments, the T cells can be activated by binding to an agent that activates CD3ζ.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100, which is incorporated herein by reference in its entirety). Other antibodies which bind to the same epitopes as any of the above described antibodies can also be used.

In some embodiments, the T cells are activated by administering phorbol myristate acetate (PMA) and ionomycine. In some embodiments, the T cells are activated by administering an appropriate antigen that induces activation and then expansion. In some embodiments, PMA, ionomycin, and/or appropriate antigen are administered with CD3 induce activation and/or expansion.

In general, the activating agents used in the present invention includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441, which is incorporated herein by reference in its entirety), decabodies (Stone, E., et al., Journal of Immunological Methods (2007) 318, 88-94, which is incorporated herein by reference in its entirety) and other domain antibodies (Holt, L. J., et al., Trends Biotechnol. (2003), 21, 11, 484-490, which is incorporated herein by reference in its entirety). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In some embodiments, one or more binding sites of the CD3ζ agents may be a bivalent proteinaceous artificial binding molecule such as a dimeric lipocalin mutein (i.e., duocalin). In some embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In some embodiments, the activating agent (e.g., CD3-binding agents) can be present in a concentration of about 0.1 to about 10 pg/ml. In some embodiments, the activating agent (e.g., CD3-binding agents) can be present in a concentration of about 0.2 μg/ml to about 9 μg/ml, about 0.3 μg/m1 to about 8 μg/ml, about 0.4 μg/ml to about 7 μg/ml, about 0.5 μg/ml to about 6 μg/ml, about 0.6 g/ml to about 5 μg/ml, about 0.7 g/ml to about 4 μg/ml, about 0.8 μg/ml to about 3 μg/ml, or about 0.9 μg/ml to about 2 μg/ml. In some embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In some embodiments, the CD3-binding agents can be present in a concentration of 1 μg/ml.

In some embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

Polynucleotide and/or Polypeptide Transfer in T cells

In some embodiments, the T cells are genetically modified by introducing polynucleotides and/or polypeptide (e.g., a site-specific nuclease, a guide RNA, an RNAi molecule, an antisense oligonucleotide, a CAR, or polynucleotides encoding the same) into the cells. The T cells can be genetically modified after stimulation/activation. In some embodiments, the T cells are modified within 12 hours, 16 hours, 24 hours, 36 hours, or 48 hours of stimulation/activation. In some embodiments, the cells are modified within 16 to 24 hours after stimulation/activation. In some embodiments, the T cells are modified within 24 hours.

In order to genetically modify the T cell, the polynucleotides and/or polypeptide (e.g., a site-specific nuclease, a guide RNA, an RNAi molecule, an antisense oligonucleotide, a CAR, a BAFT protein, or polynucleotides encoding the same) must be transferred into the cell. Polynucleotide and/or polypeptide transfer may be via viral, non-viral gene delivery methods, or a physical method. Suitable methods for polynucleotide and/or polypeptide delivery for use with the current methods include any method known by those of skill in the art, by which a polynucleotide and/or polypeptide can be introduced into an organelle, cell, tissue or organism.

In various embodiments, polypeptides or polynucleotides (e.g., a site-specific nuclease, a guide RNA, an RNAi molecule, an antisense oligonucleotide, a CAR, a BAFT protein, or polynucleotides encoding the same) described in the present invention are introduced to the T cell via a recombinant vector.

In some embodiments, the vector is a viral vector. Suitable viral vectors that can be used in the present invention include, but are not limited to, a retroviral vector, a lentiviral vector, an adenoviral vector, an adeno-associated viral (AAV) vector, a herpes viral vector, or a baculoviral vector. In one specific embodiment, the viral vector is a lentiviral vector. In one specific embodiment, the viral vector is a retroviral vector.

In some embodiments, the T cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity. In some embodiments, the T cells may be removed from the subject and transfected ex vivo using the polynucleotides (e.g., expression vectors) of the invention. In some embodiments, the T cells obtained from the subject can be transfected or transduced with the polynucleotides (e.g., expression vectors) of the invention and then administered back to the subject.

In some embodiments, polynucleotides and/or polypeptides are transferred to the cell in a non-viral vector (e.g., a transposon, a plasmid). The non-viral vector may be an RNA and/or DNA vector.

Nucleic acid vaccines may also be used to transfer polynucleotides into the T cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In some embodiments, the polynucleotide(s) is operatively linked to at least one regulatory element for expression of the gene product (e.g., a site-specific nuclease, a guide RNA, an RNAi molecule, a CAR, a BAFT protein). The regulatory element can be capable of mediating expression of the gene product in the host cell (e.g., modified T cell). Regulatory elements include, but are not limited to, promoters, enhancers, initiation sites, polyadenylation (polyA) tails, IRES elements, response elements, and termination signals. In some embodiments, the regulatory element regulates expression of the gene product. In some embodiments, the regulatory element increased the expression of the gene product. In some embodiments, the regulatory element increased the expression of the gene product once the host cell (e.g., modified T cell) is activated. In some embodiments, the regulatory element decreases expression of the gene product. In some embodiments, the regulatory element decreases expression of the gene product once the host cell (e.g., modified T cell) is activated.

In various embodiment, polypeptides or polynucleotides (e.g., a CAR, a signaling molecule, site-specific nuclease, an RNAi molecule or an antisense oligonucleotide, a BAFT protein, or polynucleotides encoding the same) are introduced into the modified T cell using a physical means. Suitable physical means include, but are not limited to, electroporation, microinjection, magnetofection, ultrasound, a ballistic or hydrodynamic method, or a combination thereof.

Electroporation is a method for polynucleotide and/or polypeptide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In some embodiments, cell wall-degrading enzymes, such as pectin-degrading enzymes, can be employed to render the T cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

In some embodiments, the present invention provides a method of modifying a gene in a cell, comprising introducing into the cell a site-specific nuclease via electroporation. In some embodiments, Cas9 protein and one or more guide RNAs are combined to form a ribonucleoprotein (RNP) complex. In some embodiments, the guide RNA comprises a nucleotide sequence as set forth in any one of SEQ ID NOs: 1-9, 29-34 and 36-42, or a nucleotide sequence having at least 80% identity thereof. In some embodiments, the guide RNA comprises the nucleotide sequence of SEQ ID NO: 29, SEQ ID NO: 34, SEQ ID NO: 36 or SEQ ID NO: 41. An exemplary protocol is detailed in Example 14 in the Examples section below. It was shown in Example 14 that this method results in higher targeting efficiency compared to application of CRISPR/Cas9 for gene editing using viral delivery of Cas9 and guide RNA. Using this method to edit a gene (e.g., Regnase-1), the editing efficiency was greater than 90% based on deep sequencing results. The RNP electroporation method also had lower toxicity for T cells compared to DNA electroporation.

Another method for polynucleotide and/or polypeptide transfer includes injection. In some embodiments, a polypeptide, a polynucleotide or viral vector may be delivered to a cell, tissue, or organism via one or more injections (e.g., a needle injection). Non-limiting methods of injection include injection of a composition (e.g., a saline based composition). Polynucleotides and/or polynucleotides can also be introduced by direct microinjection. Non-limiting sites of injection include, subcutaneous, intradermal, intramuscular, intranodal (allows for direct delivery of antigen to lymphoid tissues). intravenous, intraprotatic, intratumor, intralymphatic (allows direct administration of DCs) and intraperitoneal. It is understood that proper site of injection preparation is necessary (e.g., shaving of the site of injection to observe proper needle placement).

Additional methods of polynucleotide and/or polypeptide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide and/or polypeptide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and/or polypeptide and delivered into cells by a propelling force. See e.g., U .S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

Expansion/Proliferation of T cells

After the T cells are activated and transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120, each of which is incorporated by reference in their entirety for all purposes). Other illustrative examples for agents that may be used for the expansion of T cells are agents that bind to CD8, CD45 or CD90, such as α CD8, α CD45 or α CD90 antibodies. Illustrative examples of T cell population including antigen-specific T cells, T helper cells, cytotoxic T cells, memory T cell (an illustrative example of memory T-cells are CD62L|CD8| specific central memory T cells) or regulatory T cells (an illustrative example of Treg are CD4⁺CD25⁺CD45RA⁺ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In some embodiments, the agent(s) used for expansion (e.g., IL-7, IL-15) are administered at about 20 units/ml to about 200 units/ml. In some embodiments, the agent(s) used for expansion (e.g., IL-7, IL-15) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In some embodiments, the agent(s) used for expansion (e.g., IL-7, IL-15) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In some embodiments, the agent(s) used for expansion (e.g., IL-7, IL-15) are administered at about 5 mg/ml to about 10 ng/ml. In some embodiments, the agent(s) used for expansion (e.g., IL-7, IL-15) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In some embodiments, the agent(s) used for expansion (e.g., IL-7, IL-15) are administered at about 5 ng/ml, 6 ng/ml, 7 ng/ml, 8 ng/ml, 9, ng/ml, or 10 ng/ml.

Conditions appropriate for T cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for T cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

In further embodiments, methods of the present disclosure described herein (e.g., modifying a Regnase-1 gene or gene product) may be applied to enhance a NK cell function. NK cell refers to a differentiated lymphocyte with a CD3- CD16⁺, CD3- CD56⁺, CD16⁺ CD56⁺ and/or CD57⁺ TCR- phenotype.

In some embodiments, NK cells can be isolated and/or enriched. For example, a specific subpopulation of T lymphocytes, expressing one or more markers such as, but not limited to, CD2, CD16, CD56, CD57, CD94, CD122 or a combination thereof may be selected using either positive or negative selection techniques.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety.

In some embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In some embodiments, the NK cells can be activated by, for example and not limitation, feeder cells (e.g., native K562 cells or K562 cells that are genetically modified to express 4-1BBL and cytokines such as IL15 or IL21).

In some embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In some embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

After the NK cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of NK cells can include agents that bind to CD16 or CD56, such as for example aCD16 or aCD56 antibodies. In some embodiments, the binding agent includes antibodies (see for example Hoshino et al, Blood. 1991 Dec. 15; 78(12):3232-40.). Other agents that may be used for expansion of NK cells may be IL-15 (see for example Vitale et al. 2002. The Anatomical Record. 266:87-92, which is incorporated by reference in their entirety for all purposes).

### Compositions of the Invention

Compositions of the present disclosure include, but are not limited to, cell compositions and pharmaceutical compositions.

In one aspect, the present disclosure provides modified T cells produced by the methods described herein. Modified T cells of the present disclosure have enhanced expansion and/or persistence and/or anti-tumor or anti-infection function. In some embodiments, the T cell is a CD8⁺ αβ TCR T cell. In some embodiments, the T cell is a CD4⁺ αβ TCR T cell. In some embodiments, the T cell is a regulatory T cell (Treg). In some embodiments, the T cell is engineered to express a T cell receptor or chimeric antigen receptor (CAR).

In one aspect, the present disclosure provides a pharmaceutical composition comprising the modified T cells prepared using the methods described herein and a pharmaceutically acceptable carrier and/or excipient. Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified T cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified T cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In some embodiments, the compositions are formulated for parenteral administration, e.g., intravascular (intravenous or intraarterial), intraperitoneal, intratumoral, intraventricular, intrapleural or intramuscular administration. In some embodiments, the composition is reconstituted from a lyophilized preparation prior to administration.

In some embodiments, the modified T cells may be mixed with substances that adhere or penetrate prior to their administration, e.g., but not limited to, nanoparticles.

In another aspect, provided herein are isolated polynucleotides for use in the methods and compositions described herein. In some embodiments, an isolated polynucleotide of the present disclosure comprises the nucleotide sequence of any one of SEQ ID NOs: 1-9, 29-34 and 36-42, or a variant having at least 50%, at least 55%, at least 60%, at least 65%, at least 70%, at least 75%, at least 80%, at least 85%, at least 90%, or at least 95% sequence identity with any one of SEQ ID NOs: 1-9, 29-34 and 36-42.

In one embodiment, an isolated polynucleotide of the present disclosure comprises the nucleotide sequence of SEQ ID NO: 1.

In one embodiment, an isolated polynucleotide of the present disclosure comprises the nucleotide sequence of SEQ ID NO: 2.

In one embodiment, an isolated polynucleotide of the present disclosure comprises the nucleotide sequence of SEQ ID NO: 29.

In one embodiment, an isolated polynucleotide of the present disclosure comprises the nucleotide sequence of SEQ ID NO: 34.

In one embodiment, an isolated polynucleotide of the present disclosure comprises the nucleotide sequence of SEQ ID NO: 36.

In one embodiment, an isolated polynucleotide of the present disclosure comprises the nucleotide sequence of SEQ ID NO: 41.

In various embodiments, the isolated polynucleotide is a DNA molecule. In various embodiments, the isolated polynucleotide is an RNA molecule. In some embodiments, the isolated polynucleotide may comprise modified nucleotides. Modified nucleotides may include any of those known to a skilled artisan.

In some embodiments, the isolated polynucleotide is a guide RNA. In some embodiments, the guide RNA is a single guide RNA (sgRNA).

In some embodiments, the isolated polynucleotide is or is encoded in a recombinant vector. The recombinant vector may be a viral vector or non-viral vector, such as those described herein.

### Therapeutic Methods

In one aspect, the present disclosure provides a method of treating a disease in a subject in need thereof, including administering to the subject an effective amount of the modified T cells or the pharmaceutical composition described herein. The modified T cells may be prepared using the methods as disclosed above.

In some embodiments, the modified T cells are autologous cells. In some embodiments, the modified T cells are allogeneic cells.

The treatment may be carried out by isolating a T cell or a population of T cells from the subject (e.g., for autologous cell transfer) or a donor (e.g., for allogeneic cell transfer); modifying a Regnase-1 gene or gene product in the T cell(s) such that the expression and/or function of Regnase-1 in the T cell(s) is reduced or eliminated; and administering an effective amount of the modified T cells to the subject. Optionally, the T cell(s) may be activated and/or expanded before or after the modification step. In some embodiments, one or more additional genes or gene products, including but not limited to Ptpn2, Socs1, Agps, Rc3h1, Rcor1, Ireb2, Vti1a, or Pex13, may be modified alone or in combination with Regnase-1 and/or Batf in the T cell(s) such that the expression and/or function of the modified gene(s) in the T cell(s) is reduced or eliminated.

The treatment may be carried out by isolating a T cell or a population of T cells from the subject (e.g., for autologous cell transfer) or a donor (e.g., for allogeneic cell transfer); increasing the expression of Batf gene and/or enhancing the function of BATF protein in the T cell; and administering an effective amount of the modified T cells to the subject. Optionally, the T cell(s) may be activated and/or expanded before or after the modification step. In some embodiments, the method further comprises modifying one or more additional genes or gene products in the T cell such that the expression and/or function of the additional gene(s) or gene product(s) in the T cell is reduced or eliminated, wherein the additional gene(s) or gene product(s) are selected from Regnase-1 (REGNASE-1, Zc3h12a, MCPIP1), Ptpn2, Socs1, Agps, Rc3h1, and Rcor1. In some embodiments, the additional gene(s) or gene product(s) is Regnase-1 (REGNASE-1, Zc3h12a, MCPIP1).

In some embodiments, the disease being treated by the therapeutic methods of the present disclosure is a cancer or an infectious disease.

The terms “cancer” and “cancerous” refer to or describe the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of cancer can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at interne website of Merck Manuals); and SEER Program Coding and Staging Manual 2016, each of which are incorporated by reference in their entirety for all purposes.

In some embodiments, the cancer is a solid tumor. Non-limiting examples of solid tumors that may be treated by the therapeutic methods of this present disclosure include ovarian cancer, lung cancer (e.g., non-small cell lung squamous cell carcinoma, adenocarcinoma, large cell carcinoma types, and small cell lung cancer), breast cancer, colon cancer, kidney cancer (e.g., renal cell carcinomas), bladder cancer, liver cancer (e.g., hepatocellular carcinoma), stomach cancer, cervical cancer, prostate cancer, testicular cancer, pancreatic cancer, nasopharyngeal cancer, thyroid cancer (e.g., thyroid papillary carcinoma), skin cancers (e.g., melanoma), brain cancer (e.g., glioma, astrocytoma and medulloblastoma) and sarcoma (e.g., osteosarcoma and Ewing's sarcoma). In some embodiments, the cancer is a melanoma, colon cancer, breast cancer, or brain cancer.

In some embodiments, the cancer is a blood cancer. In some embodiments, the blood cancer is a lymphoma, leukemia, or multiple myeloma. Non-limiting examples of leukemia that may be treated by the therapeutic methods of this present disclosure include acute lymphoblastic leukemia (ALL), B-cell acute lymphoblastic leukemia, T-cell acute lymphoblastic leukemia, acute non-lymphocytic leukemia (ANLL), acute myeloblastic leukemia (AML), acute promyelocytic leukemia (APL), acute monocytic leukemia, acute erythroleukemia leukemia, acute megakaryoblastic leukemia, chronic myelogenous leukemia (CIVIL), hairy cell leukemia, and chronic lymphocytic leukemia (CLL). Non-limiting examples of lymphoma that may be treated by the therapeutic methods of this present disclosure include lymphoplasmacytic lymphoma, small lymphocytic lymphoma (SLL), splenic marginal zone B cell lymphoma, nodal marginal band B cell lymphoma, prolymphocytic white blood, follicular lymphoma (FL), mantle cell lymphoma (MCL), Burkitt's lymphoma, and diffuse large B-cell lymphoma (DLBCL) Hodgkin lymphoma, lymphoblastic lymphoma, anaplastic large cell lymphoma (ALCL), subcutaneously T cell lymphoma, peripheral T-cell lymphoma, angioimmunoblastic ball lymphoma, angiocentric lymphoma (nasal type T cell lymphoma), malignant lymphoma, Hodgkin's lymphoma, non-Hodgkin's lymphoma, follicular lymphoma, and other lymphomas of lymphoid origin and skin.

In some embodiments of therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of the modified T cells. It is also contemplated that a variety of doses will be effective to improve in vivo effector function of the modified T cells.

In some embodiments, composition comprising the T cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of T cells will depend on the therapeutic use for which the composition is intended for.

Modified T cells may be administered multiple times at dosages listed above. The T cells may be allogeneic, syngeneic, xenogeneic, or autologous to the patient undergoing therapy.

The compositions and methods described in the present disclosure may be utilized in conjunction with other types of therapy for cancer, such as chemotherapy, surgery, radiation, gene therapy, and so forth.

The compositions and methods described in the present disclosure may be used to treat an infectious disease. Infectious diseases are well known to those skilled in the art, and non-limiting examples include but are not limited to infections of viral etiology such as HIV, influenza, Herpes, viral hepatitis, Epstein Bar, polio, viral encephalitis, measles, chicken pox, Papilloma virus; infections of bacterial etiology such as pneumonia, tuberculosis, syphilis; or infections of parasitic etiology such as malaria, trypanosomiasis, leishmaniasis, trichomoniasis, amoebiasi s.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the invention can be combined with other therapies that block inflammation (e.g., via blockage of ILL INFα/β, IL6, TNF, IL23, etc.).

The methods and compositions of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CD1d-fusion proteins, CD dimers or larger polymers of CD either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the disclosure can be combined with additional immunotherapies and therapies. For example, when used for treating cancer, the compositions of the invention can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the T cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present invention include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel , abciximab; antimigratory agents; anti secretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the therapeutic methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Identification of Regnase-1 as a Major Negative Regulator of CD8⁺ T cell Antitumor Responses using in vivo CRISPR-Cas9 Mutagenesis Screening

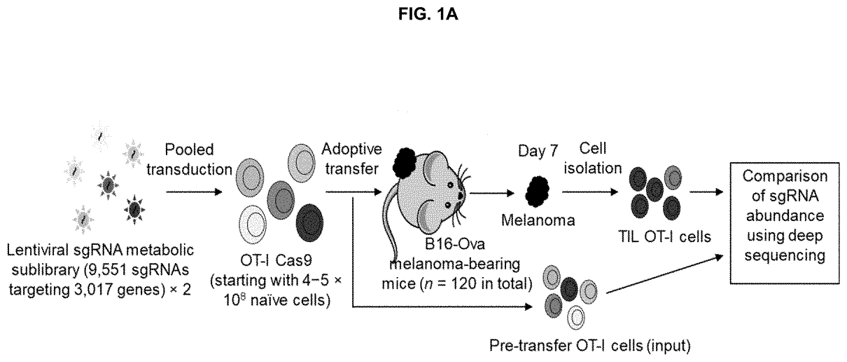

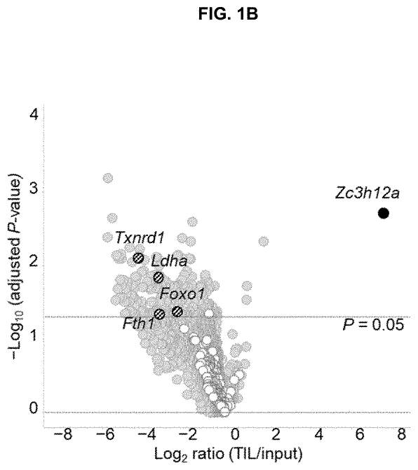

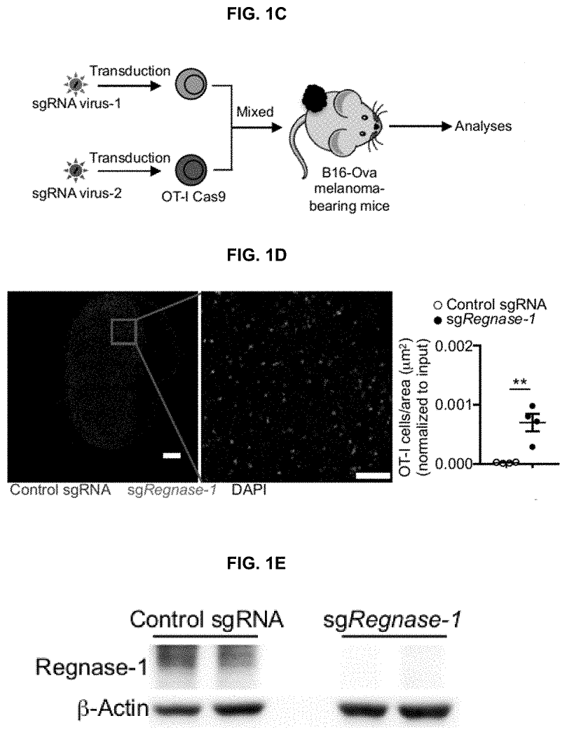

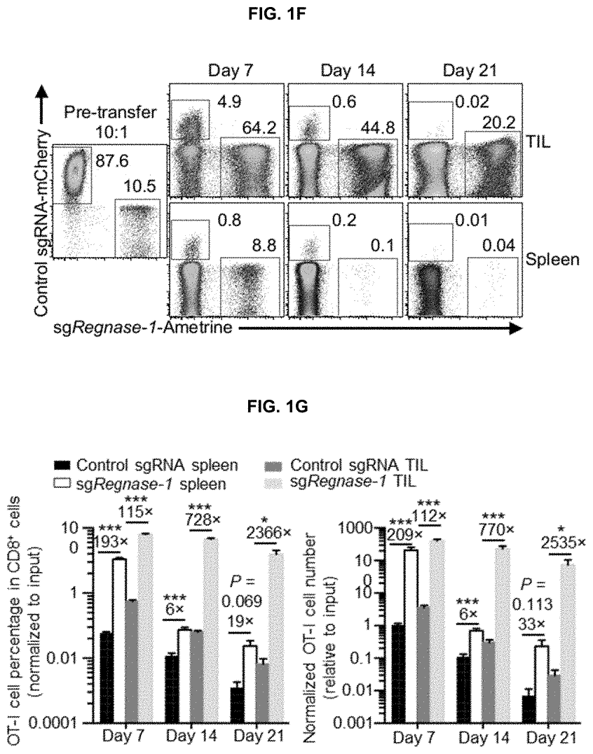

To systematically investigate metabolism-associated factors whose loss can improve T cell accumulation within tumors, a pooled CRISPR-Cas9 mutagenesis screening approach was developed in a mouse melanoma ACT model (FIG. 1A), which has been used successfully in a short hairpin RNA (shRNA)-based screening¹⁷.

First, constitutive Rosa26-Cas9-expressing mice¹⁸ (The Jackson Laboratory) were crossed with OT-I T-cell receptor transgenic mice¹⁹ (The Jackson Laboratory) to express Cas9 in ovalbumin-specific CD8⁺ T cells (OT-I cells) that recognize B16 melanoma cells expressing ovalbumin as a surrogate tumor antigen (B16-Ova cells)²⁰. All mice were kept in a specific pathogen-free facility in the Animal Resource Center at St. Jude Children's Research Hospital. Animal protocols were approved by the Institutional Animal Care and Use Committee of St. Jude Children's Research Hospital. Naïve Cas9-expressing OT-I cells were isolated from the spleen and peripheral lymph nodes (PLNs) of Cas9-OT-I mice using naive CD8α⁺ T cell isolation kit (Miltenyi Biotec 130-096-543) according to manufacturer's instructions. Purified na-ve OT-I cells were activated in vitro for 18 h with 10 μg/m1 anti-CD3 (2C11; Bio X Cell), 5 μg/ml anti-CD28 (37.51; Bio X Cell) before viral transduction.

Next, two sub-libraries of sgRNAs were developed, each consisting of 9,051 sgRNAs targeting 3,017 cell metabolism-related genes (6 sgRNAs per gene altogether) encoding metabolic enzymes, small molecule transporters, and metabolism-related transcriptional regulators²¹, as well as 500 non-targeting control sgRNAs in a lentiviral vector containing Ametrine fluorescent protein. Viral transduction was performed by spin-infection at 800 g at 25° C. for 3 h with 10 μg/ml polybrene (Sigma). Cells were continued to culture with human IL-2 (20 UI/ml; PeproTech), mouse IL-7 (25 ng/ml; PeproTech) and IL-15 (12.5 ng/ml; PeproTech) for 3-4 days. Transduced cells were sorted using a Reflection (i-Cyt) before adoptive transfer into recipients.

After transduction of the sgRNA library and in vitro activation and expansion to allow gene editing to occur, sgRNA-transduced OT-I cells were adoptively transferred into B16-Ova melanoma-bearing mice. Seven days later, OT-I cells in tumor-infiltrating lymphocytes (TILs) were purified by flow cytometry, and library representation in TILs and pre-transfer (input) OT-I cells was examined by deep sequencing of sgRNA cassette. sgRNAs capable of improving ACT were expected to be enriched in tumor-infiltrating OT-I cells. After merging the quantification results from two sub-libraries, candidate genes were ranked based on the average enrichment of their 6 gene-specific sgRNAs in tumor-infiltrating OT-I cells relative to input (log₂ ratio (TIL/input); adjusted P<0.05).

There were a total of 218 genes significantly depleted (by less than −1.0 log₂ ratio (TIL/input); adjusted P<0.05) in the screening, indicative of impaired survival or expansion in the absence of these putative positive regulators of T cell responses, including Txnrd1²² (log₂ ratio (TIL/input)=−4.23), Ldha²³ (log₂ ratio (TIL/input)=−3.30), Fth1²⁴ (log₂ ratio (TIL/input)=−3.25), and Foxol^{25,26} (log₂ ratio (TIL/input)=−2.39) that are important regulators of T cell survival and expansion (FIG. 1B). Strikingly, Zc3h12a (also known as Regnase-1, encodes Regnase-1) was the mostly highly enriched gene in this screening (FIG. 1B), with all of its targeting sgRNAs ranked at top 6 of the most enriched sgRNAs, suggesting that Regnase-1 could be a major negative regulator of antitumor responses.

For the lentiviral sgRNA metabolic library CRISPR-Cas9 mutagenesis screening, the following methods were used.

Lentiviral and retroviral sgRNA vector design: The lentiviral sgRNA vector was generated from lentiGuide-puro vector by replacing the “EF-1α PuroR” fragment with a mouse PGK promoter-driven Ametrine (or GFP or mCherry) fluorescent protein. The retroviral sgRNA vector was generated from pLMPd-Amt vector²⁷ by replacing the miR30 shRNA cassette with the U6 promoter driven gRNA cassette from the lentiGuide-puro vector.

Lentiviral sgRNA metabolic library construction: The gene list of mouse metabolic library was based on the reported human metabolic genes²¹. A total of 6 gRNAs were designed for each mouse metabolic gene according to previously-published selection criteria²⁸ and were split into two sub-libraries (AAAQ05 and AAAR07), each containing 500 non-targeting controls. sgRNAs were designed by using the online sgRNA design tool (portal s.broadinstitute. org/gpp/public/analysi s-tool s/sgrna-design). Oligonucleotides containing the guide sequence were synthesized (Custom Array), PCR amplified, and cloned into the recipient vector via a Golden Gate cloning procedure, including 5 μl Tango Buffer (ThermoFisher), 5 μl DTT (10 mM stock); 5 μl ATP (10 mM stock); 500 ng vector, pre-digested with Esp3I, gel-extracted, and isopropanol-precipitation purified; 100 ng insert PCR product; 1 μl Esp3I (ThermoFisher ER0452); 1 μl T7 ligase (Enzymatics, 3,000 Units/L6020L); and water, up to 50 and incubated in cycle (5 min at 37° C. and 5 min at 20° C.) for 100 times. The product was then purified by isopropanol precipitation and electroporated into STBL4 cells (Life Technologies 11635018). The distribution of the library was determined by Illumina sequencing.

Selected sgRNAs used in this study were as follows:

non-targeting control sgRNA:
(SEQ ID NO: 10)
ATGACACTTACGGTACTCGT;
sgRegnase-1:
(SEQ ID NO: 1)
AAGGCAGTGGTTTCTTACGA;
sgRegnase-1 #2:
(SEQ ID NO: 2)
GGAGTGGAAACGCTTCATCG;
sgBatf:
(SEQ ID NO: 3)
AGAGATCAAACAGCTCACCG;
sgBatf #2:
(SEQ ID NO: 4)
AGGACTCATCTGATGATGTG;
sgPtpn2:
(SEQ ID NO: 5)
AAGAAGTTACATCTTAACAC;
sgPtpn2 #2:
(SEQ ID NO: 6)
CACTCTATGAGGATAGTCAT;
sgSocs1:
(SEQ ID NO: 7)
TGATGCGCCGGTAATCGGAG;
sgSocs1 #2:
(SEQ ID NO: 8)
TGGTGCGCGACAGTCGCCAA;
sgAgps:
(SEQ ID NO: 9)
GTACCAATGAGTGCAAAGCG;
sgRc3h1:
(SEQ ID NO: 42)
GGTAGAGGGTTACTACCCGG.

In vivo screening: Lentivirus was produced by co-transfecting HEK293T cells with the lentiviral metabolic library plasmids, psPAX2 (Addgene plasmid # 12260) and pCAG4-Eco. At 48 h after transfection, virus was harvested and froze at −80° C. Four hundred to five hundred million na-ve Cas9-expressing OT-I cells were isolated from 8-14 Cas9-OT-I mice and transduced at a MOI of 0.3 to achieve ˜20% transduction efficiency. After viral transduction, cells were cultured with human IL-2 (20 IU/ml; PeproTech), mouse IL-7 (25 ng/ml; PeproTech) and IL-15 (12.5 ng/ml; PeproTech) for 4 days. Transduced cells expressing Ametrine were sorted using a Reflection sorter (i-Cyt), and an aliquot of 5×10⁶ transduced OT-I cells was saved as “input” (˜500× cell coverage per sgRNA). Transduced OT-I cells (5×10⁶ cells per recipient) were i. v. transferred into mice at day 14 after B16-Ova melanoma engraftment. Sixty recipients were randomly divided into 3 groups as biological replicates in each sub-library screening. At 7 days after adoptive transfer, transferred Ametrine⁺OT-I cells were recovered from the tumor pooled from 20 recipients per sample using a Reflection sorter (i-Cyt). On average, 5×10⁵ OT-I cells per sample (˜50× cell coverage per sgRNA) were recovered for further analysis.

Sequencing library preparation: Genomic DNA was extracted by using the DNeasy Blood & Tissue Kits (Qiagen 69506). Primary PCR was performed by using the KOD Hot Start DNA Polymerase (Millipore 71086) and the following pair of Nextera NGS primers (Nextera NGS-F: TCGTCGGCAGCGTCAGATGTGTATAAGAGACAGttgtggaaaggacgaaacaccg (SEQ ID NO: 11); Nextera NGS-R: GTCTCGTGGGCTCGGAGATGTGTATAAGAGAC AGccactttttcaagttgataacgg (SEQ ID NO: 12). Primary PCR products were purified using the AMPure XP beads (Beckman A63881). A second PCR was performed to add adaptors and indexes to each sample. Hi-Seq 50-bp single-end sequencing (Illumina) was performed.

Data processing: For data analysis, FastQ files obtained after sequencing were demultiplexed using the HiSeq Analysis software (Illumina). Single-end reads were trimmed and quality-filtered using the CLC Genomics Workbench v11 (Qiagen) and matched against sgRNA sequences from the sgRNA metabolic library. Read counts for sgRNAs were normalized against total read counts across all samples. For each sgRNA, the fold change (log₂ ratio) for enrichment was calculated between each of the biological replicates and the input experiment. Gene ranking was based on the average enrichment among replicates in representation of 6 individual corresponding sgRNAs (combing two sub-libraries) in sgRNA metabolic sub-libraries, respectively. The gene level false discovery rate adjusted P-value was calculated among multiple sgRNAs of each gene, using a paired two-tailed t-test between log₂ transformed average normalized read counts of tumor samples and those of input sample, and a value of less than 0.05 was considered to be statistically significant.

For tumor-infiltrating lymphocyte (TIL) isolation, B16-Ova melanoma was excised, minced and digested with 0.5 mg/ml Collagenase IV (Roche)+200 IU/ml DNase I (Sigma) for 1 h at 37° C., and then passed through 70 μm filters to remove undigested tumor. TILs were then isolated by density-gradient centrifugation over Percoll (Life Technologies).

For flow cytometry analysis, cells were stained in PBS (Gibco) containing 2% (wt/vol) BSA (Sigma). Surface proteins were stained for 30 min on ice. Intracellular staining was performed with Foxp3/Transcription Factor Staining Buffer Set according to manufacturer's instructions (eBioscience). Intracellular staining for cytokines was performed with fixation/permeabilization kit (BD Biosciences). Caspase-3 staining was performed using instructions and reagents from the “Active Caspase-3 Apoptosis Kit” (BD Biosciences). BrdU staining was performed using instructions and reagents from the “APC BrdU Flow Kit” (BD Biosciences). 7-AAD (Sigma) or Fixable viability dye (eBioscience) was used for dead cell exclusion. The following antibodies were used: anti-CD27 (LG.7F9), anti-IFN-γ (XMG1.2), anti-TNFα (MAb11), anti-IL-2 (JES6-5H4), anti-CD69 (H1.2F3), anti-CD25 (PC61.5), anti-CD62L (DREG-56), anti-CXCR3 (CXCR3-173), anti-KLRG1 (2F1), anti-ICOS (7E.17G9), anti-Lag3 (C9B7W), anti-PD-1 (J43), anti-CTLA4 (1B8), anti-TOX (TXRX10) (all from eBioscience); anti-GzmB (QA16A02), anti-CD43a (1B11), anti-CD49a (HMα1), anti-CD44 (IM7), anti-Ki-67 (16A8), anti-CD127 (A7R34) (all from Biolegend); anti-BrdU (3D4), anti-active caspase-3 (C92-605), anti-pH2A.X-S139 (N1-431), anti-Slamf6 (13G3) (all from BD Biosciences); anti-BATF (D7C5), anti-Bim (C34C5), anti-TCF-1 (C63D9) (all from Cell Signaling Technology); anti-CD8α (53-6.7) (from SONY); anti-CD62L (MEL-14) (from TONBO Bioscience) and anti-Tim3 (215008) (from R&D). To monitor cell division, lymphocytes were labeled with CellTrace Violet (CTV; Life Technologies). For staining mitochondria, lymphocytes were incubated for 30 min at 37° C. with 10 nM Mito Tracker Deep Red (Life Technologies) or 20 nM TMRM (tetramethylrhodamine, methyl ester; ImmunoChemistry Technologies) after staining surface markers. Flow cytometry data were analyzed using Flowjo 9.9.4 (Tree Star).

For biological experiment (non-omics) analyses, data were analyzed using Prism 6 software (GraphPad) by two-tailed paired Student's t-test or two-tailed unpaired Student's t-test, or one-way ANOVA with Newman-Keuls's test. Two-way ANOVA was performed for comparing tumor growth curves. Log-rank (Mantel-Cox) test was performed for comparing mouse survival curves. P<0.05 was considered significant. Data are presented as mean±s.d. or mean±s.e.m.

### Example 2

### Validation of Regnase-1 effects

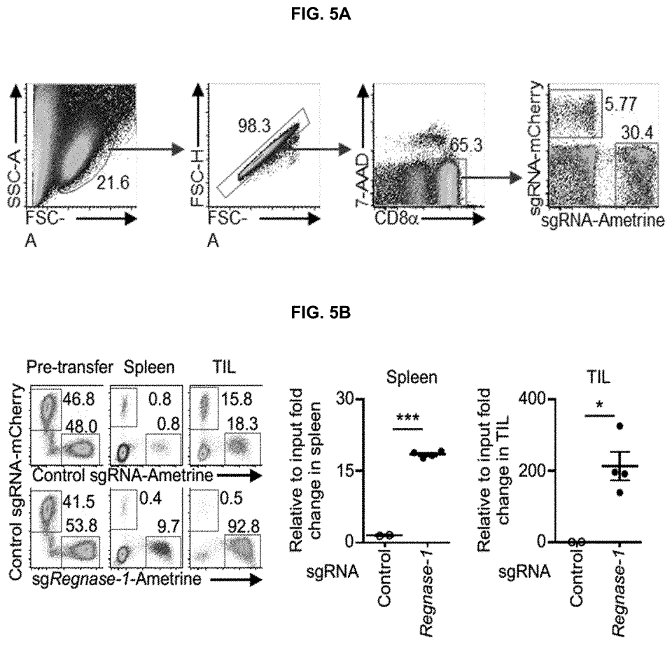

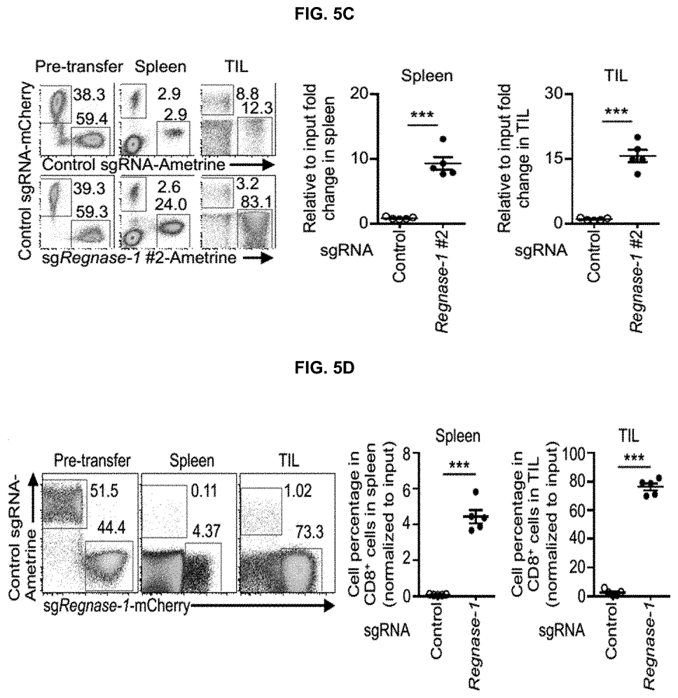

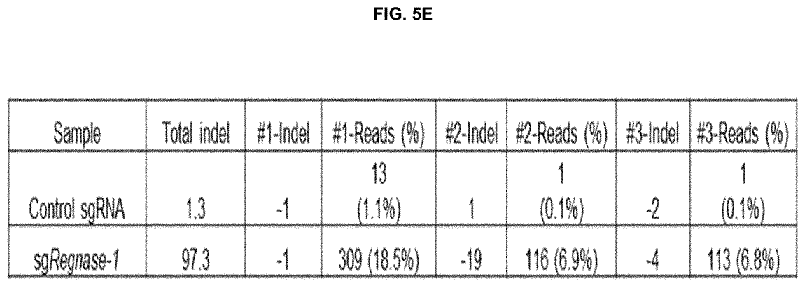

To validate the screening results, an in vivo dual transfer system was developed to compare the relative accumulation of OT-I cells transduced with sgRNA lentiviral vectors expressing distinct fluorescent proteins in the same tumor-bearing host (FIG. 1C). To exclude the possibility that the different fluorescent proteins could affect cell accumulation, OT-I cells transduced with lentiviral vectors encoding the same control sgRNA were mixed but with different fluorescent proteins. At 7 days after adoptive transfer into B16-Ova melanoma-bearing host, their relative proportions in both the spleen and tumor were preserved as the pre-transfer mixture (FIGS. 5A-5C, upper panels). To verify the inhibitory effect of Regnase-1 on CDS⁺ T cell accumulation, OT-I cells were transduced with two different sgRNAs targeting Regnase-1. The relative proportion of Regnase-1-null OT-I cells was drastically increased in both the spleen and tumor after adoptive transfer (FIGS. 5B and 5C). Altering the fluorescent protein reporters for sgRegnase-1 yielded similar results (FIG. 5D), and this further excluded the contribution from the different fluorescent proteins. Imaging analysis identified significantly more Regnase-1-null OT-I cells within tumors than wild-type controls (FIG. 1D). Analysis of guide targeting efficacy showed 97.3% indel events in sgRegnase-1-transduced cells isolated from tumors as compared to 1.3% in control sgRNA-transduced cells (FIG. 5E), and immunoblot analysis further validated the loss of Regnase-1 expression in tumor-infiltrating OT-I cells transduced with Regnase-1 sgRNA (FIG. 1E). Next, the persistence of Regnase-1-null OT-I cells was examined at days 7, 14 and 21 after transfer, since adoptively transferred effector CDS⁺ T cells are known to show limited long-term persistence in tumor-bearing hosts'. While both the proportion and number of wild-type OT-I cells declined drastically over time in the spleen and tumor, Regnase-1-null OT-I cells had markedly better persistence, especially in the tumor (FIGS. 1F and 1G). The persistence advantage of Regnase-1-null OT-I cells over wild-type controls in the tumor became more pronounced at days 14 (˜700x) and 21 (˜2,000x) as compared to day 7 (˜100x), while in the spleen, Regnase-1-null OT-I cells showed modest advantage at days 14 (˜5x) and 21 (˜20x) compared with day 7 (˜200x) (FIGS. 1F and 1G). Therefore, loss of Regnase-1 endows tumor-specific CD8⁺ T cells with greatly improved accumulation and long-term persistence, preferentially within the tumor.

For the protein immunoblot analysis, cells were lysed in RIPA buffer (ThermoFisher 89900), resolved in 4-12% Criterion™ XT Bis-Tris Protein Gel (Bio-Rad 3450124) and transferred to PVDF membrane (Bio-Rad 1620177). Membranes were blocked using 5% BSA for 1 h and then incubated for overnight with anti-MCPIP1 antibody (604421) (R&D), anti-BATF (D7C5) (Cell Signaling Technology), anti-PTPN2 (E-11) (Santa Cruz Biotechnology), anti-SOCS1 (E-9) (Santa Cruz Biotechnology), anti-Hsp90 (MAB3286) (R&D) and anti-β-actin (8H10D10) (Cell Signaling Technology). Membranes were washed 6 times with TBST and then incubated with 1:5,000 diluted HRP conjugated anti-mouse IgG (W4021) (from Promega) for 1 h. Following another 6 times of washes with TBST, the membranes were imaged using the ODYSSEY Fc Analyzer (LI-COR).

For imaging, B16-Ova melanomas were fixed in PBS containing 2% PFA, 0.3% Triton-100 and 1% DMSO for 24 h prior to cryoprotection in 30% sucrose. Cryosections were blocked with 1% BSA and 0.05% Tween-20 in TBS (20 mM Tris, pH 8.0, 100 mM NaCl) for 1 h at room temperature prior to overnight incubation in blocking buffer containing the following antibodies; anti-mCherry (Biorbyt orb11618), anti-GFP (Rockland Immuno 600-401-215), anti-TCF-7 (C63D9) (Cell Signaling Technology 2203), and anti-Tom20 (2F8.1) (Millipore MABT166). Slides were washed in TBS before application of AF488, Cy3 or AF647 secondary antibodies (Jackson Immuno) for 1 h at room temperature prior to mounting with Prolong Diamond hardset media containing DAPI (Thermofisher). Widefield fluorescence microscopy was performed using a motorized Nikon TiE inverted microscope equipped with a 20×Plan Apo 0.75NA objective, standard DAPI, FITC and TRITC filter sets, and an EMCCD camera (Andor). The entire tissue section was stitched based on the DAPI fluorescent signal and the subsequent large images were analyzed using NIS Elements software (Nikon Instruments). Images were segmented per channel, and further refined using a spot identification algorithm to identify single cells and positional information within the tumor. The number of cells per square area was determined following manual delineation of the tumor border. Analysis of transcription factor localization was performed using a Marianis spinning disk confocal microscope (Intelligent Imaging Innovations) equipped with a 100×1.4 NA objective and Prime 95B sCMOS camera, and analyzed using Slidebook software (Intelligent Imaging Innovations).

### Example 3

### Evaluation of Regnase-1-Deficient CD8⁺ T Cells in Tumor Models

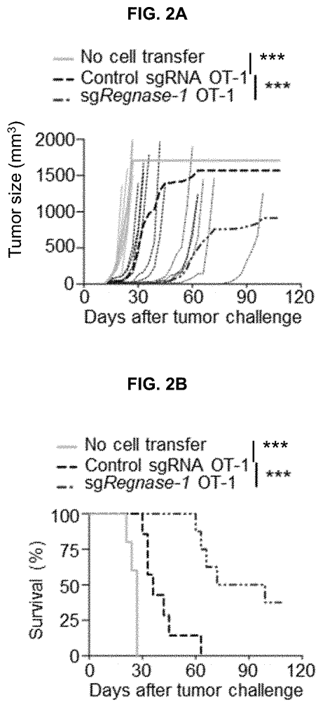

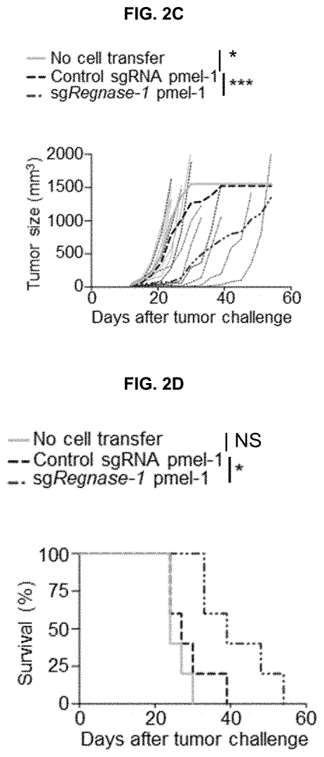

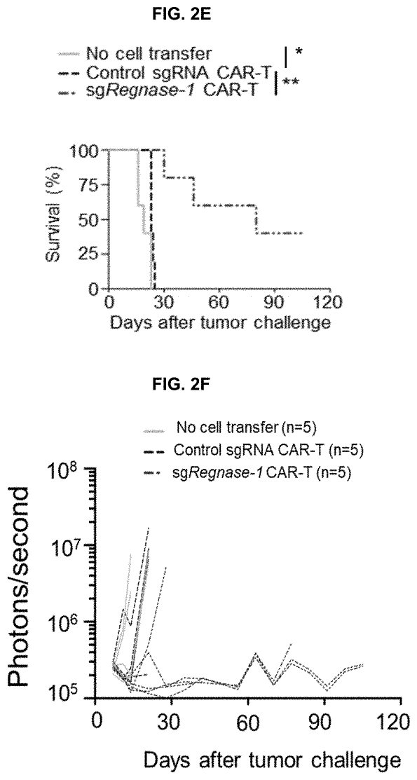

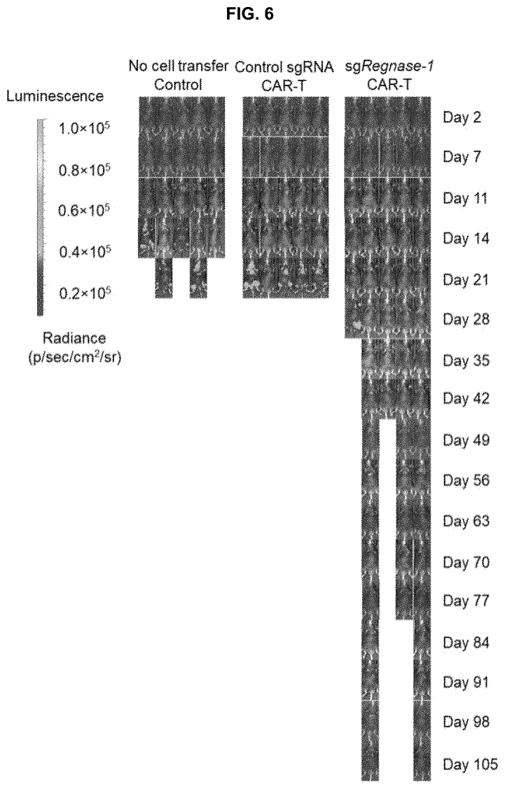

Given the improved longevity and drastically increased cellularity of Regnase-1-null CD8⁺ T cells within tumors, the efficacy of Regnase-1-null CD8⁺ T cells in ACT was assessed in three tumor models. First, the therapeutic efficacy of Regnase-1-null OT-I cells was determined against B16-Ova melanoma, an aggressive tumor that is difficult to treat²⁹. While wild-type OT-I cells had modest therapeutic effects against melanoma, as expected¹⁷, Regnase-1-null OT-I cells showed much stronger antitumor effects, evidenced by markedly inhibited tumor growth and increased survival of melanoma-bearing mice (FIGS. 2A and 2B). Next, CD8⁺ T cells from pmel-1 T-cell receptor-transgenic mice³⁰ (pmel-1 cells, which recognize the endogenous melanoma antigen gp100; from the Jackson Laboratory) crossed with Cas9-expressing mice¹⁸ were used. Although wild-type pmel-1 cells were unable to effectively inhibit B16-F10 melanoma growth, Regnase-1-null pmel-1 cells had greatly increased therapeutic efficacy (FIGS. 2C and 2D). Last, to assess the ability of Regnase-1 deletion to enhance the efficacy of CAR-T cells against leukemia, Cas9-expressing mice¹⁸ were crossed with transgenic mice that express CARs (consisting of anti-human CD19 (huCD19) scFv fragments, mouse CD8 transmembrane domain and mouse 4-1BB-CD3ζ signaling tail; provided by Dr. Terrence Geiger at St. Jude Children's Research Hospital) under the CD2 promoter to generate CAR-T cells. Aggressive mouse BCR-ABL1⁺B progenitor acute lymphoblastic leukemia (Ph⁺B-ALL) cells³¹ were also generated that express huCD19 as a surrogate tumor antigen and luciferase for in vivo imaging (huCD19-Ph⁺B-ALL). Strikingly, as compared to wild-type CD8⁺ CAR-T cells, Regnase-1-null CD8⁺ CAR-T cells showed much stronger therapeutic efficacy against huCD19-Ph⁺B-ALL as indicated by the greatly increased survival (FIG. 2E) and reduced luciferase signals measured by in vivo imaging (FIG. 2F and FIG. 6). Collectively, Regnase-1 deletion markedly enhances the efficacy of ACT against both solid and blood cancers.

For adoptive T cell transfer for tumor therapy, B16-Ova cells (2×10⁵; provided by Dr. Dario Vignali at University of Pittsburgh) or B16-F10 cells (2×10⁵; ATCC) were injected subcutaneously into female C57BL/6 mice (7-10 weeks age; from The Jackson Laboratory). At day 12, mice bearing tumors of similar size were randomly divided into 3 groups (5-8 mice per group), and sgRNA-transduced OT-I cells (5×10⁶) (for the treatment of B16-Ova melanomas) or pmel-1 (5×10⁶) (for the treatment of B16-F10 melanomas) were injected intravenously. Tumors were measured every three days with digital calipers and tumor volumes were calculated by the formula: Length×Width×[(Length×Width) \hat{ } 0.5]×π/6³². Death was defined as the point at which a progressively growing tumor reached 15 mm in the longest dimension. For the treatment of huCD19-Ph⁺B-ALL, mice engrafted with huCD19-Ph⁺B-ALL (1×10⁶; provided by Dr. Terrence Geiger at St. Jude Children's Research Hospital) were treated at day 7 with sgRNA-transduced CDS⁺ CAR-T cells (5×10⁶). Mice were imaged using the Xenogen imaging system (Caliper Life Science).

### Example 4

### Gene Expression Profiling in Regnase-1-Deficient CD8⁺ T cells

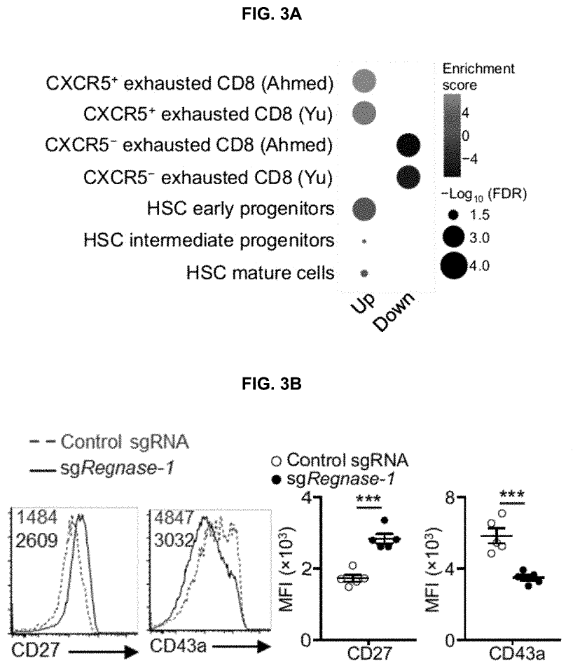

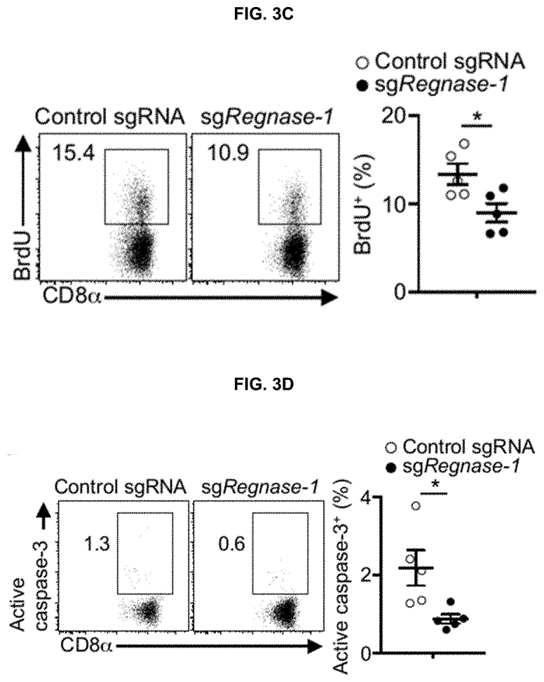

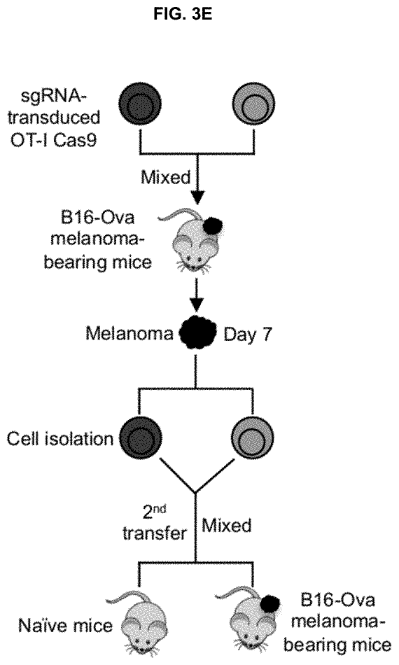

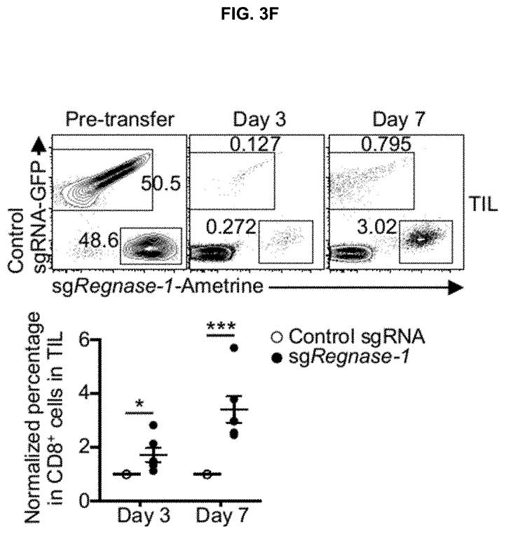

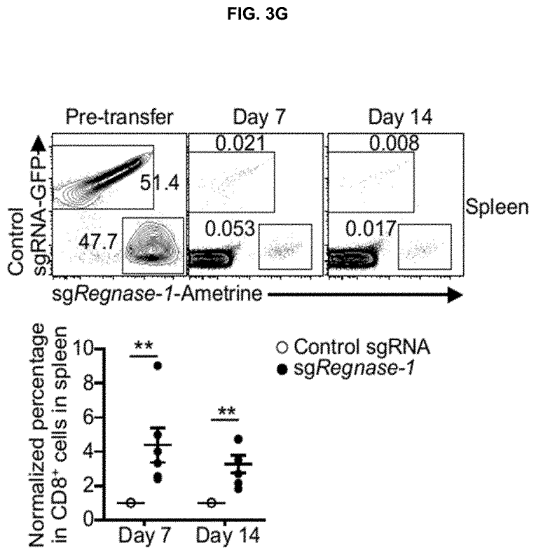

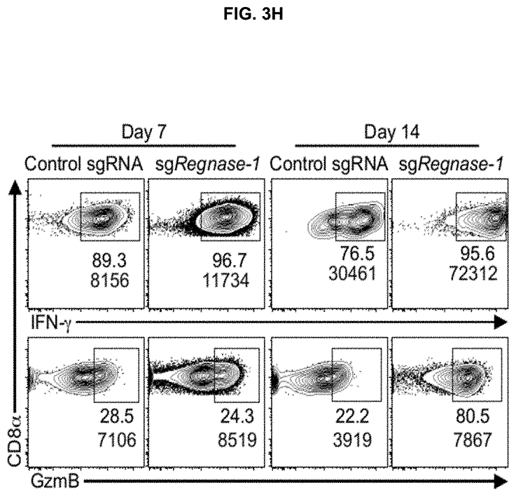

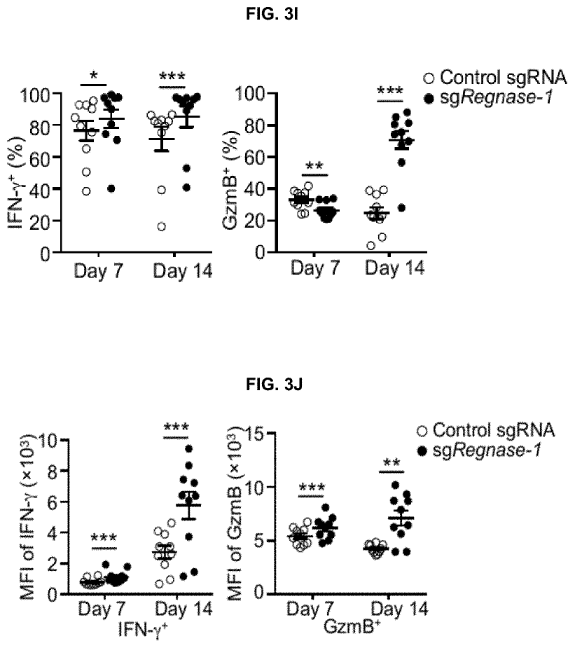

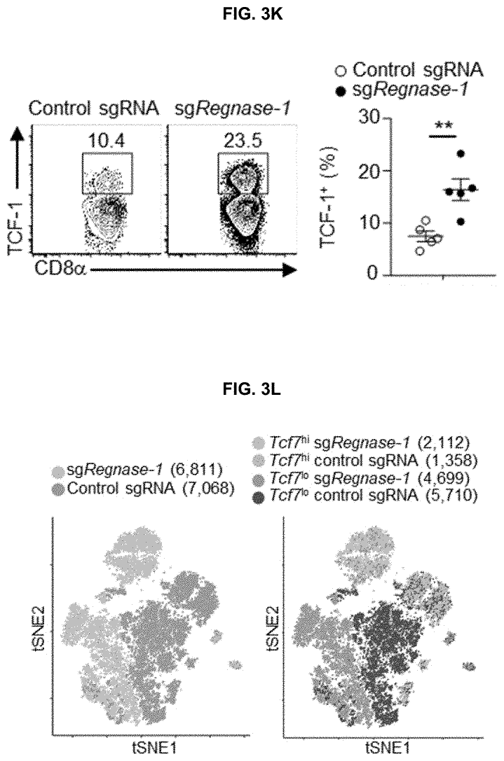

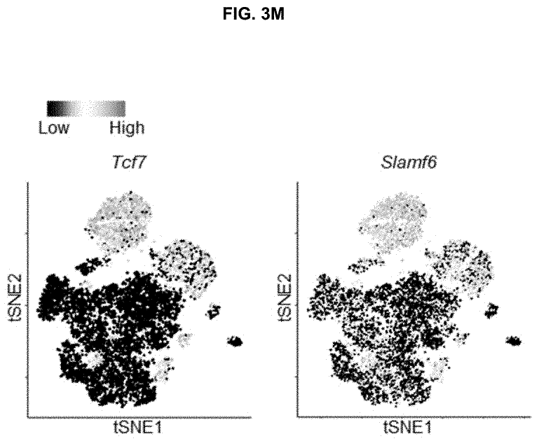

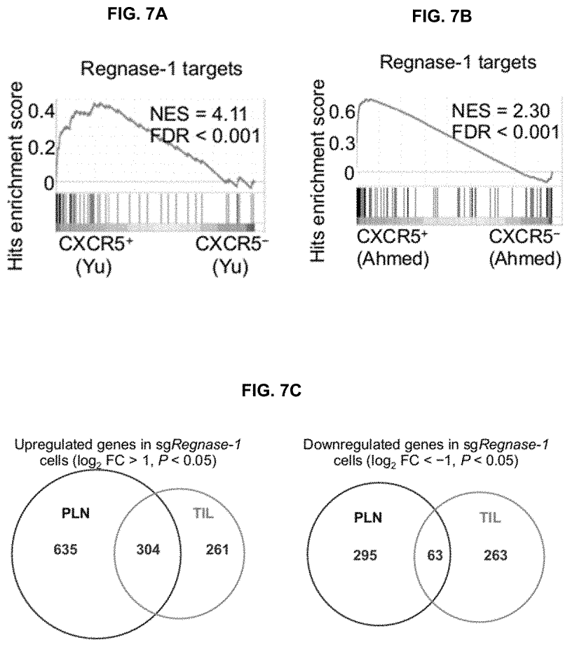

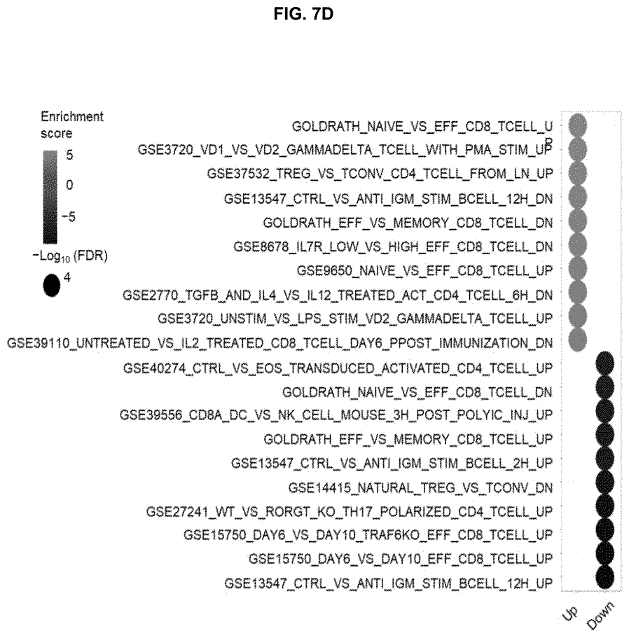

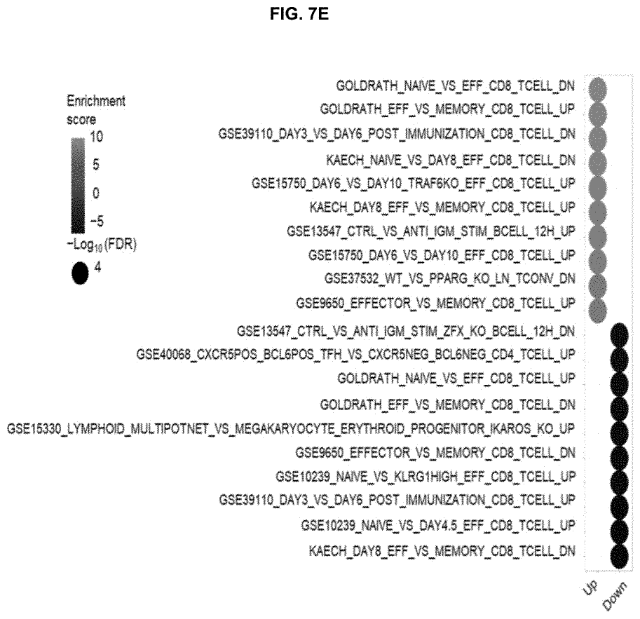

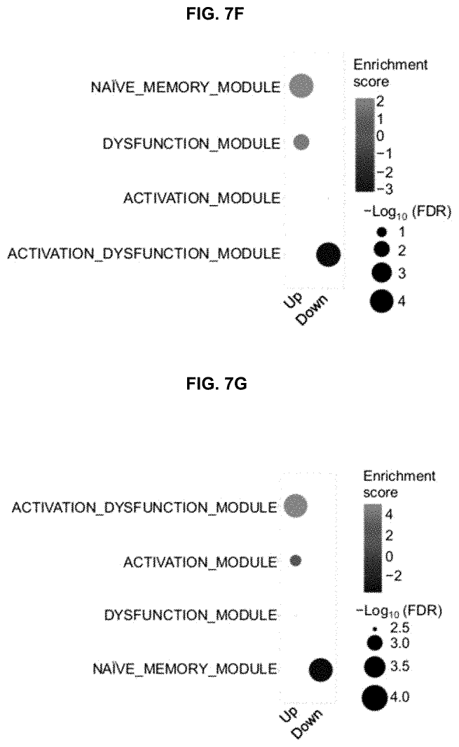

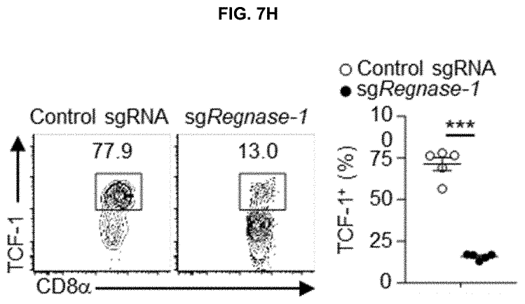

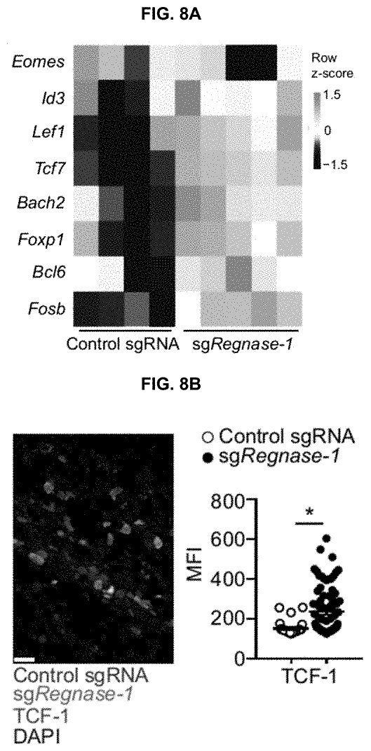

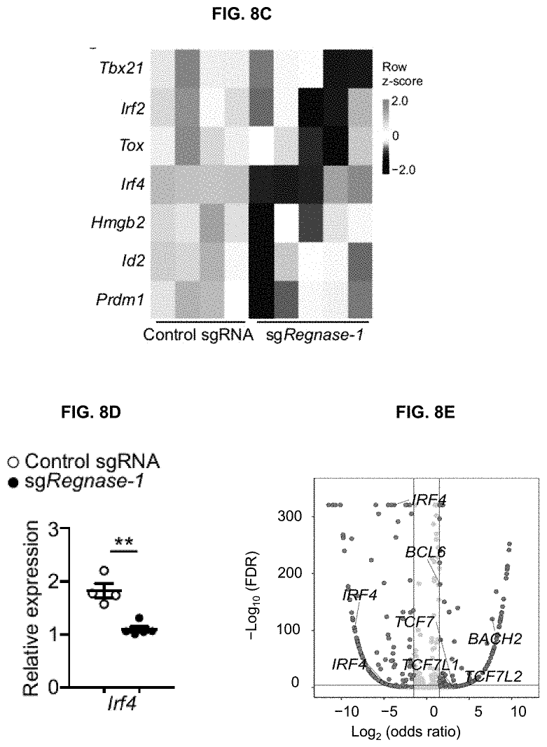

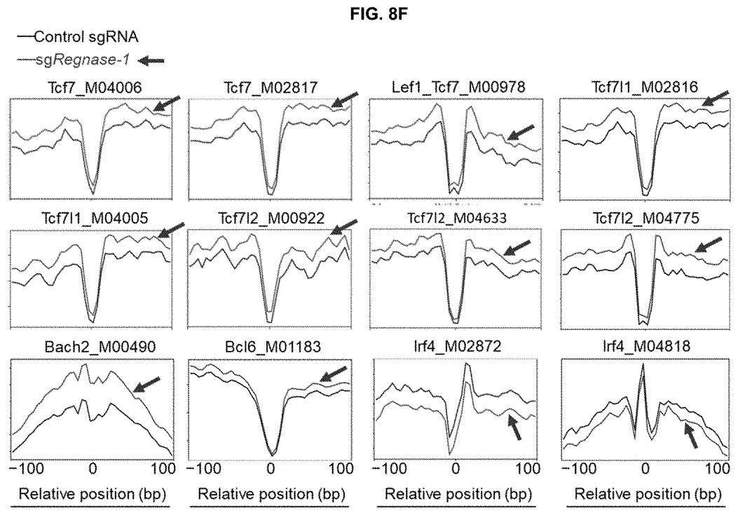

To systemically identify the differences in immune processes and functional states between Regnase-1-null and wild-type OT-I cells, RNA-Sequencing (RNA-Seq) and bioinformatic analyses of cells isolated from the in vivo dual transfer system were performed to address cell-intrinsic effects. The association of the enhanced long-term persistence of Regnase-1-null TILs with stem-like properties by performing gene set enrichment analysis (GSEA) was determined using gene signatures representative of memory-like CDS⁺ T cells (CXCR5⁺vs CXCR5⁻ exhausted cells) in chronic infection^{7,8}, as well as hematopoietic stem cells (HSCs)³³. As compared to wild-type controls, tumor-infiltrating Regnase-1-null OT-I cells had highly enriched gene signatures associated with stem-like CDS⁺ T cells and HSC progenitors (FIG. 3A). Consistent with this notion, gene targets repressed by Regnase-1 (i.e. those upregulated upon its deletion) were significantly enriched in stem-like CDS⁺ T cells (FIGS. 7A and 7B), suggesting that these cells may have low Regnase-1 activity. Interestingly, transcriptional profiling revealed marked differences between tumor-infiltrating and peripheral (from lymph nodes) Regnase-1-null OT-I cells (FIG. 7C), raising the possibility that Regnase-1-null effector CDS⁺ T cells undergo specific reprogramming in the TME. To test this idea and obtain an unbiased view of immune processes regulated by Regnase-1, GSEA was performed using “immunologic signatures” gene sets. Tumor-infiltrating Regnase-1-null OT-I cells were highly enriched for gene signatures associated with memory formation and naive cells (FIG. 7D), consistent with the enrichment of stem-like signatures described above (FIG. 3A). In contrast, peripheral Regnase-1-null OT-I cells were enriched with genes related to effector cells or those downregulated in memory formation or naive cells (FIG. 7E). Moreover, to further support these findings, GSEA was performed using previously defined gene modules associated with different functional states of CDS⁺ T cells in tumor immunity'. While tumor-infiltrating Regnase-1-null OT-I cells were enriched with naive or memory module, peripheral Regnase-1-null cells were associated with activation-associated but not naive or memory module (FIGS. 7F and 7G). Consistent with the bioinformatics inference, tumor-infiltrating Regnase-1-null OT-I cells had increased expression of the memory or naive T cell-associated marker CD27 and reduced expression of the effector cell-associated marker CD43a, as compared to wild-type controls (FIG. 3B)^{34,35}. Additionally, tumor-infiltrating Regnase-1-null OT-I cells expressed higher levels of transcription factors associated with na-ve or memory CD8⁺ T cells, including Id3, Lef1, Tcf7 (encodes TCF-1), Bach2, Foxp1, Bcl6, and Fosb³⁶⁻⁴° (FIGS. 8A and 8B), but had lower expression of effector or exhausted CD8⁺ T cell-associated transcription factors including Irf2, Irf4, Hmgb2, Id2, and Prdm1 (encodes Blimp1)^{37,41,45} (FIGS. 8C and 8D), and not significantly altered expression of Eomes, Tbx21 and Tox (FIGS. 8A and 8C). Given the extensive transcriptional changes, chromatin accessibility was next measured using ATAC-Seq (assay for transposase accessible chromatin using sequencing⁴⁶) of tumor-infiltrating Regnase-1-null and wild-type OT-I cells, and motif searches were performed on accessible regions of assembled ATAC-Seq reads to explore enriched transcription factor binding motifs. Compared to wild-type controls, Regnase-1-null cells showed significant enrichments in TCF-1, Bach2 and Bc16 motifs, but downregulated the IRF4 motif (FIGS. 8E and 8F). These results suggest that Regnase-1-null effector CD8⁺ T cells are reprogrammed in the TME and acquire naive/memory cell-associated gene expression programs.

For gene expression profiling, gene set enrichment analysis (GSEA) and weighted gene co-expression network analysis (WGCNA), control sgRNA- and sgRegnase-1-transduced OT-I cells (n=4-5 biological replicates each group) were isolated from the tumors or PLN of the hosts of the in vivo dual color transfer assay, and analyzed with RNA-Seq. For RNA-Seq, RNA was quantified using the Quant-iT RiboGreen assay (Life Technologies) and quality checked by 2100 Bioanalyzer RNA 6000 Nano assay (Agilent) or LabChip RNA Pico Sensitivity assay (PerkinElmer) prior to library generation. Libraries were prepared from total RNA with the TruSeq Stranded Total RNA Library Prep Kit according to the manufacturer's instructions (Illumina, PN 20020595). Libraries were analyzed for insert size distribution on a 2100 BioAnalyzer High Sensitivity kit (Agilent Technologies) or Caliper LabChip GX DNA High Sensitivity Reagent Kit (PerkinElmer.) Libraries were quantified using the Quant-iT PicoGreen dsDNA assay (Life Technologies) or low pass sequencing with a MiSeq nano kit (Illumina). Paired end 100 cycle sequencing was run on the HiSeq 4000 (Illumina). The raw reads were trimmed for adapter sequences using Trimmomatic v.0.36 using parameters ILLUMINACLIP : adapter. fa: 2 :30: 10 LEADING:10 TRAILING:10 SLIDINGWINDOW : 4 : 18 MINLEN:32, followed by mapping to mm9 reference genome downloaded from gencode release M1 (www.gencodegenes.org/mouse/releases.html) using star v.2.5.2b. with default parameters. Reads were summarized at gene level using python script htseq-count. Differential expression analysis was performed using R package DEseq2 v. 1.18.1. Donor-derived T cells isolated from the tumor-bearing mice that received the individual transfer of sgRNA-transduced OT-I cells (n=3-4 biological replicates each group) were analyzed using microarrays (Affymetrix Mouse Clariom S Assay), which were collected in the following three batches: (a) Control sgRNA-, sgRegnase-1-, and sgBatf/Regnase-1-transduced TIL OT-I cells (n=4 replicates each group); (b) Control sgRNA- and sgBatf-transduced TIL OT-I cells (n=3 replicates each group); and (c) Control sgRNA-, sgRegnase-1-, sgPtpn2-, sgPtpn2I Regnase-1-, sgSocs1-, and sgSocs//Regnase-1-transduced TIL OT-I cells (n=4 replicates each group). For microarray, the expression signals were summarized robust multi-array average algorithm Affymetrix Expression Console v1.1, followed by differential expression analysis performed using R package limma v.3.34.9. All the plots were generated using R package ggplot2 v.2.2.1. Differentially expressed transcripts were identified by ANOVA (Partek Genomics Suite version 6.5), and the Benjamini-Hochberg method was used to estimate the false discovery rate (FDR) as described'. Differentially expressed (DE) genes were defined by |log2 FC|>0.5; P<0.05. To analyze microarray samples from the two different batches (a) and (b) as described above, the batch effect was corrected using removeBatchEffect function implemented in R package limma v.3.34.9. GSEA was performed as described⁶² using the “Hallmark” database. For GSEA using manually curated gene signatures from public datasets, microarray dataset (GSE84105)⁷ was used for generating “CXCR5⁺exhausted CD8 (Ahmed)” and “CXCR5⁻ exhausted CD8 (Ahmed)” gene signatures (<5% FDR); as total upregulated and downregulated genes were more than 200, genes were ranked by their log₂ fold change of expression in CXCR5⁺ vs CXCR5⁻ comparison and used top 200 upregulated genes as “CXCR5⁺exhausted CD8 (Ahmed)” and top 200 downregulated genes as “CXCR5⁻ exhausted CD8 (Ahmed)”. RNA-Seq data (GSE76279)⁴⁸ was processed using DEseq2 R package v 1.16.1 to generate “CXCR5⁺exhausted CD8 (Yu)” and “CXCR5⁻ exhausted CD8 (Yu)” using the similar strategy as the other signatures above. Similarly, gene signatures of different subsets of hematopoietic stem cells (HSCs) were generated³³. Weighted gene co-expression network analysis (WGCNA) was performed as described^{12,49}. Briefly, the analysis was performed using WGCNA R package v. 1.66. Total mRNA co-expression clusters were defined using DE genes defined as described above. Pearson correlation matrix was calculated for each experiment followed by an adjacency matrix calculation by raising the correlation matrix to a power of 10 to meet the scale-free topology criterion⁴⁹. Co-expression clusters were defined by hybrid dynamic tree cutting method with minimum height for merging module set at 0.2, as described⁸⁵. A consensus trend for each co-expression cluster was defined based on the first principal component, and cluster membership was defined as Pearson correlation between individual genes and the consensus trend of the co-expression cluster. Genes were assigned to the most correlated co-expression cluster with cutoff of r≥0.7, as described⁸⁵. RNA-Seq and microarray data have been deposited into the GEO series database (www. ncbi .nlm.nih.gov/geo/query/acc.cgi?acc=GSE126072, token for access: ofafckgkxlelxux).

For ATAC-Seq library preparation, tumor-infiltrating sgRNA-transduced OT-I cells were collected in the following two batches: (a) Control sgRNA- and sgRegnase-1-transduced OT-I cells (n=4 biological replicates each group) were isolated from tumor-bearing mice using the in vivo dual color transfer assay; (b) Control sgRNA-, sgRegnase-1-, sgBatf- and sgBatf/Regnase-1-transduced TIL OT-I cells (n=2-4 replicates each group) were isolated from the tumor-bearing mice that received the individual transfer of sgRNA-transduced OT-I cells. Sorted T cells were incubated in 50 μl ATAC-Seq lysis buffer (10 mM Tris-HCl, pH 7.4, 10 mM NaCl, 3 mM MgCl₂, 0.1% IGEPAL CA-630) on ice for 10 min. Resulting nuclei were pelleted at 500 g for 10 min at 4° C. Supernatant was carefully removed with a pipette and discarded. The pellet was resuspended in 50 μl transposase reaction mix (25 ₁1.1 2× TD buffer, 22.5 μl nuclease-free water, 2.5 μl Transposase) and incubated for 30 min at 37° C. After the reaction, the DNA was cleaned up using the Qiagen MinElute kit. The barcoding reaction was run using the NEBNext HiFi kit based on manufacturer's instructions and amplified for 5 cycles according to Buenrostro et al.⁴⁶ using the same primers. Ideal cycle numbers were determined from 5 μl (of 50 μl) from the previous reaction mix using KAPA SYBRFast (Kapa Biosystems) and 20 cycle amplification on an Applied Biosystems 7900HT. Optimal cycles were determined from the linear part of the amplification curve and the remaining 45 μl of PCR reaction was amplified in the same reaction mix using the optimal cycle number.

ATAC-Seq analysis was performed as described previously⁵⁰. Briefly, 2×100 bp paired-end reads obtained from all samples were trimming for Nextera adapter by cutadapt (version 1.9, paired-end mode, default parameter with “-m 6-O 20”) and aligned to mouse genome mm9 (NCBIM37 from Sanger) by BWA (version 0.7.12-r1039, default parameter)⁵¹, duplicated reads were then marked with Picard (version 2.6.0-SNAPSHOT) and only non-duplicated proper paired reads have been kept by samtools (parameter “-q 1-F 1804” version 1.2)⁵². After adjustment of Tn5 shift (reads were offset were offset by +4 bp for the sense strand and -5 bp for the antisense strand) reads were separated into nucleosome free, mononucleosome, dinucleosome, trinucleosome as described⁴⁶ by fragment size and generated bigwig files by using the center 80 bp of fragments and scale to 30×10⁶ nucleosome free reads. Reasonable nucleosome free peaks and pattern of mono-, di-, tri-nucleosome on IGV (version 2.4.13)⁵³ were observed and all 8 samples have about 10×10⁶ nucleosome free reads so the data qualities were concluded as good. Next each 2 replicates were merged to enhance peak calling on nucleosome free reads by MACS2 (version 2.1.1.20160309 default parameters with “-extsize 200-nomodel”)⁵⁴. To assure the replicability, nucleosome free regions for each genotype were first finalized as only retained a peak if it called with higher cutoff (macs2-q 0.05) in one merged sample and at least called with lower cutoff (macs2-q 0.5) in the other merged sample. There reproducible peaks were further merged between WT and KO and then nucleosome free reads from each of the 8 samples were counted by bedtools (v2.24.0)⁵⁵. To find the differential accessible regions, raw nucleosome free reads counts were first normalized using trimmed mean of M-values normalization method and applied Empirical Bayes Statistics test after linear fitting from voom package (R 3.23, edgeR 3.12.1, limma 3.26.9)⁵⁶. FDR-correct P-value 0.05 and fold change >2 were used as cutoff for more accessible regions in KO (KO Larger) or less accessible regions in KO (KO Smaller). For motif analysis, regions <0.05 fold change and P-value >0.5 were further selected as control region. At last, FIMO from MEME suite (version 4.11.3, “-thresh le-4-motif-pseudo 0.0001”)⁵⁷ have been used for scanning motif (TRANSFAC database, only included Vertebrata and not 3D structure-based) matches in the nucleosome free regions and Fisher's Exact tests have been used to test whether a motif is significant enriched for differential accessible regions compared to the control regions. ATAC-Seq data have been deposited into the GEO series database (www. ncbi .n1 m .nih. gov/geo/query/acc. cgi? acc=GSE126072, token for access: ofafckgkxlelxux).

Transcription factor binding site footprinting was performed as described previously⁵⁰. Briefly, bigwig files have been first generated by all tags of adjusted reads and normalized by autosomes reads number to 2×10⁸ reads (e.g. sample with 1×10⁸ autosome reads would be scaled to double the bigwig profile). Then average bigwig files have been generated by mean of replicates at each bp for each sample and motif matches within nucleosome free region have been used for footprinting taking the average profile across all motif matches at each bp from −100 bp from motif match centers to +100 bp. Finally, the footprinting profiles have been smoothed with 10 bp bins and plot by deeptools (v2.5.7)⁵⁸.

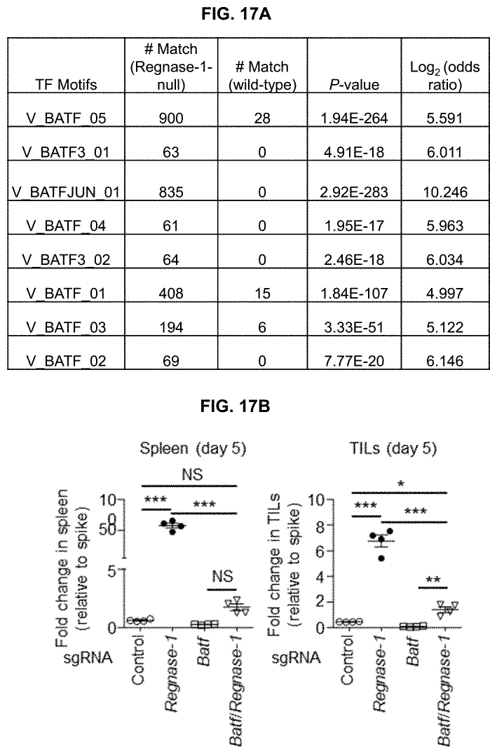

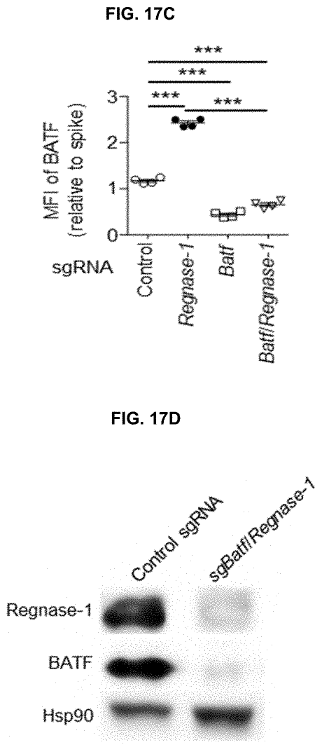

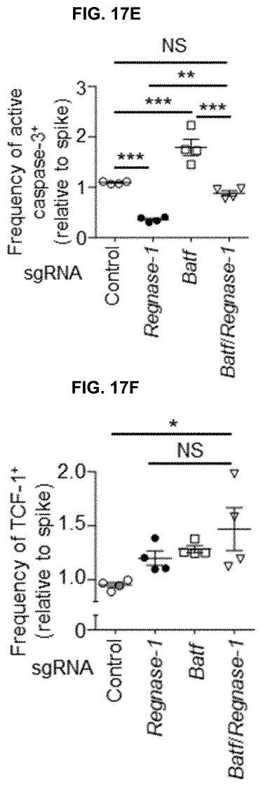

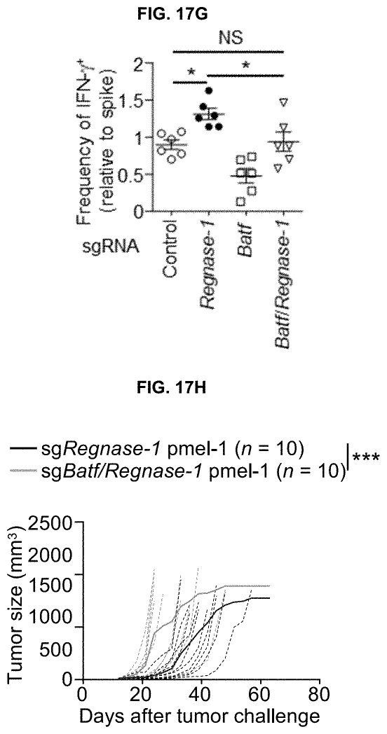

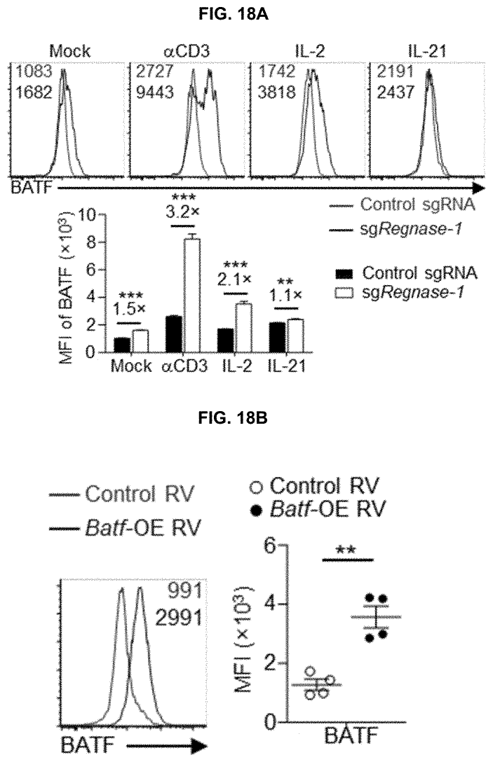

To identify the enrichment of BATF binding motifs, nucleosome-free differentially accessible regions were defined at |log2 FC|>0.5; P<0.05, and the peaks were further annotated as more or less accessible regions in Regnase-1-null OT-I cells compared to wild-type controls. For each group, differentially accessible peaks were overlapped with BATF ChIP-Seq peaks (downloaded from GSE54191⁵⁶) to identify the common regions between ATAC-Seq peaks and BATF ChIP-Seq peaks using bedtools (version 2.25.0). Finally, FIMO⁵⁹¹ from MEME suite (version 4.9.0) was used to scan the overlapping regions with TRANSFAC motifs associated with BATF to identify the number of motifs enriched in the differentially accessible regions in Regnase-1-null (shown as ‘# Match (Regnase-1-null)’ in FIG. 17A) or wild-type control samples (shown as ‘# Match (wild-type)’), and Fisher's exact test was used to test the significance of enrichment. This statistical bioinformatic method has been used successfully by us and others to circumvent cell number limitations^{50,60}.

For real-time PCR, RNA was isolated using the RNeasy Micro Kit (Qiagen 74004) following the manufacturer's instructions. RNA was converted to cDNA using the High Capacity cDNA Reverse Transcription Kit (ThermoFisher 4368813) according to manufacturer's instructions. Real time PCR was performed on the QuantStudio 7 Flex System (Applied Biosystems) using the PowerSYBR Green PCR Master Mix (ThermoFisher 4367659) and the following primers: Bcl2l1-F: GACAAGGAGATGCAGGTATTGG (SEQ ID NO: 13), Bcl2l1-R: TCCCGTAGAGATCCACAAAAGT (SEQ ID NO: 14); Ifng-F: ACAGCAAGGCGAAAAAGGATG (SEQ ID NO: 15), Ifng-R: TGGTGGACCACTCGGA TGA (SEQ ID NO: 16); Irf4-F: TCCGACAGTGGTTGATCGAC (SEQ ID NO: 17), Irf4-R: CCTCACGATTGTAGTCCTGCTT (SEQ ID NO: 18); Gzma-F: TGCTGCCCACTGTAAC GTG (SEQ ID NO: 19), Gzma-R: GGTAGGTGAAGGATAGCCACAT (SEQ ID NO: 20); Gzmb-F: CCACTCTCGACCCTACATGG (SEQ ID NO: 21), Gzmb-R: GGCCCCC AAAGTGACATTTATT (SEQ ID NO: 22); Actin-F: CGCCACCAGTTCGCCATGGA (SEQ ID NO: 23), Actin-R: TACAGCCCGGGGAGCATCGT (SEQ ID NO: 24).

### Example 5

### Reprogramming Tumor-Specific CD8⁺ T Cells by Regnase-1 Deletion

Since T cell differentiation state is an important determinant of in vivo persistence^{13,61}, the enhanced accumulation of Regnase-1-null TILs prompted determination of their differentiation status by performing gene set enrichment analysis (GSEA) using previously defined gene modules associated with different functional states of CD8⁺ T cells in tumor immunity^{1,2}. As compared to wild-type controls, tumor-infiltrating Regnase-1-null OT-I cells were enriched with naive or memory module (FIG. 7F). Additionally, gene targets repressed by Regnase-1 (i.e. those upregulated upon its deletion) were significantly enriched in memory-like CD8⁺ T cells (CXCR5⁺vs CXCR5⁻ exhausted cells) in chronic infection^{7,8} (FIGS. 7A, 7B). Consistent with the bioinformatic inference, tumor-infiltrating Regnase-1-null OT-I cells had increased expression of a key memory or na-ve T cell-associated transcription factor, TCF-1^{36,38}, as compared to wild-type controls (FIGS. 3K, 8B). Tumor-infiltrating Regnase-1-null OT-I cells also expressed higher mRNA levels of transcription factors associated with na-ve or memory CD8⁺ T cells, including Lef1, Bach2, Tcf7 (encodes TCF-1), Foxp1, Bcl6, and

Fosb^{36,38-40} (FIG. 8A), but had lower expression of effector or exhausted CD8⁺ T cell-associated transcription factors, including Irf2, Irf4 and Hmgb2⁴¹⁻⁴⁵ (FIGS. 8C, 8D), and not significantly altered expression of Eomes, Tbx21 and Tox (FIGS. 8A, 8C). Given the extensive transcriptional changes, next chromatin accessibility was measured using ATAC-Seq (assay for transposase accessible chromatin using sequencing⁴⁶) of tumor-infiltrating Regnase-1-null and wild-type OT-I cells, and performed motif searches on accessible regions of assembled ATAC-Seq reads to explore enriched transcription factor binding motifs. Compared to wild-type controls, Regnase-1-null cells showed significant enrichments in TCF-1, Bach2 and Bc16 motifs, but downregulated the IRF4 motif (FIGS. 8E, 8F). These results suggest that Regnase-1-null effector CD8⁺ T cells are reprogrammed in the TME and exhibit enhanced naive/memory cell-associated gene expression programs.

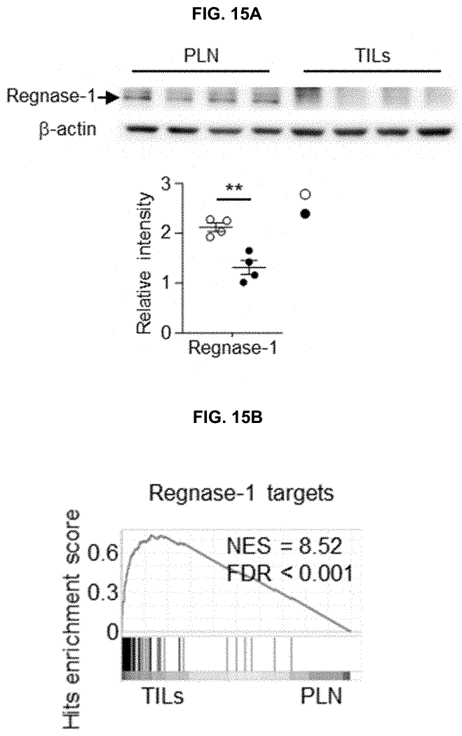

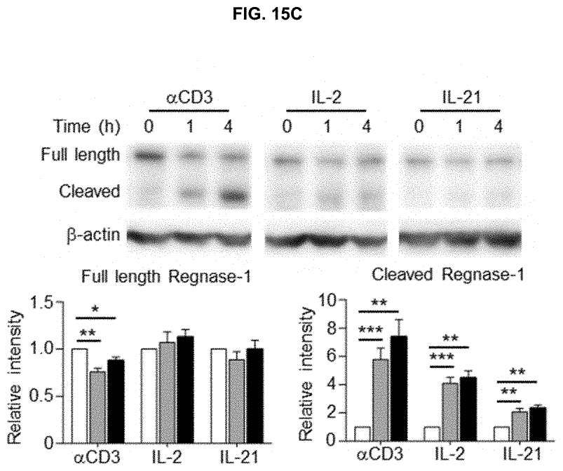

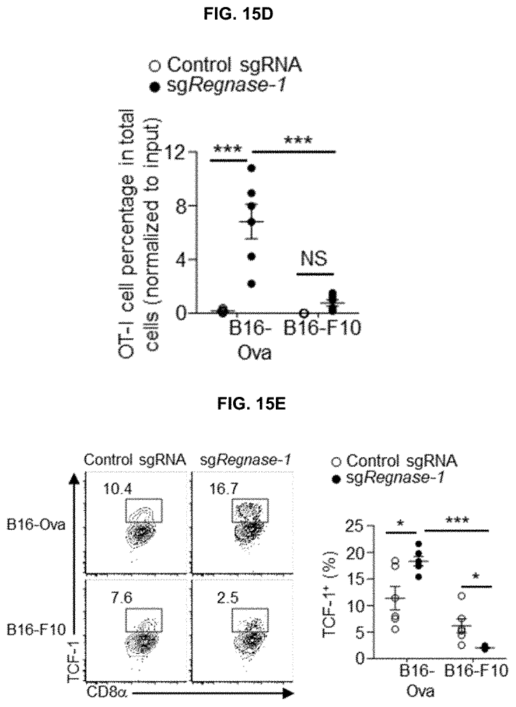

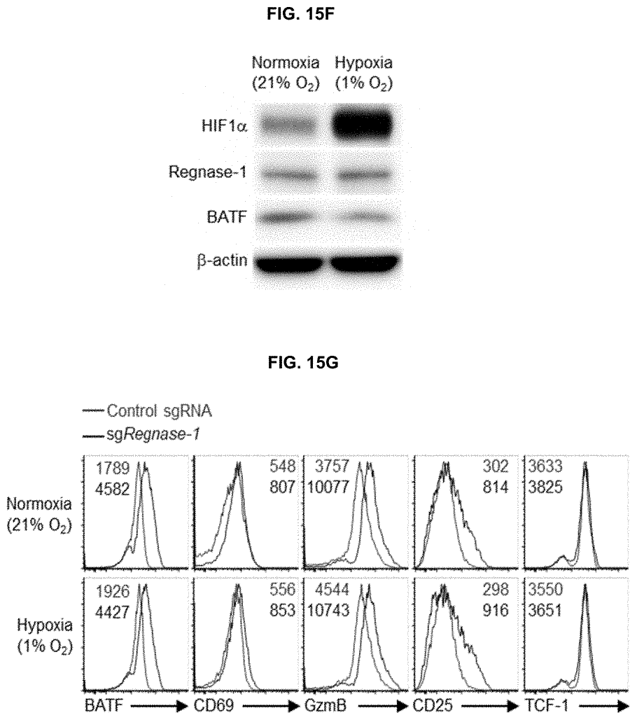

Interestingly, transcriptional profiling revealed marked differences between tumor-infiltrating and peripheral (from lymph nodes) Regnase-1-null OT-I cells (FIG. 7C). Unlike the enrichment of naive or memory module in Regnase-1-null cells in tumors (FIG. 7F), peripheral Regnase-1-null cells were associated with activation-associated but not naive or memory module (FIG. 7G), and had reduced expression of TCF-1 in the spleen of tumor-bearing mice (FIG. 7H). Given the TME-specific phenotypes of Regnase-1-null cells, the regulation of Regnase-1 expression and activity was assessed in tumor-infiltrating and peripheral wild-type OT-I cells. Tumor-infiltrating OT-I cells had lower Regnase-1 expression than peripheral OT-I cells (FIG. 15A). Additionally, gene targets repressed by Regnase-1 were significantly enriched in tumor-infiltrating OT-I cells (FIG. 15B), indicative of dampened Regnase-1 activity and in line with the elevated OT-I cell activation in TME than secondary lymphoid organs¹⁷. To explore upstream signals controlling Regnase-1 activity, Regnase-1 expression was measured in pre-activated OT-I cells upon stimulation with TCR, IL-2 or IL-21⁶². TCR engagement with anti-CD3 (αCD3) antibody decreased Regnase-1 expression and also potently induced Regnase-1 cleavage (FIG. 15C). A modest level of Regnase-1 cleavage was observed upon IL-2, and to an even lesser extent, IL-21 stimulation (FIG. 15C). To determine the role of TCR signaling in driving the reprogramming of Regnase-1-null CD8⁺ T cells in TME in vivo, Regnase-1-null OT-I cells were transferred into mice bearing either B16-Ova or B16-F10 tumor cells that express or lack the expression of the cognate antigen, respectively. Strikingly, antigen recognition was crucial in driving Regenase-1 deletion-induced CD8⁺ T cell accumulation in TILs, as evidenced by significantly reduced Regnase-1-null OT-I cells in B16-F10 melanoma-bearing mice (FIG. 15D). Antigen stimulation was also required for Regnase-1-null cells to acquire increased naive/memory cell-like phenotype, as indicated by the decreased TCF-1 expression in Regnase-1-null cells in B16-F10 compared with B16-Ova-bearing mice (FIG. 15E). While hypoxia is one of the hallmark features of the TME and regulates tumor-infiltrating CD8⁺ T cell functional states¹⁴, hypoxia did not alter Regnase-1 expression (FIG. 15F), or expression of activation or differentiation molecules, including BATF, CD69, GzmB, CD25, and TCF-1, in wild-type or Regnase-1-null OT-I cells (FIG. 15G). These results further indicate that Regnase-1-null effector CD8⁺ T cells undergo specific reprogramming in the TME, and establish Regnase-1 as an intrinsic component of the signaling processes downstream of tumor antigen-TCR stimulation but not hypoxia.

### EXAMPLE 6

### Proliferation and Survival Analyses of Regnase-1-Deficient CDs⁺ T cells

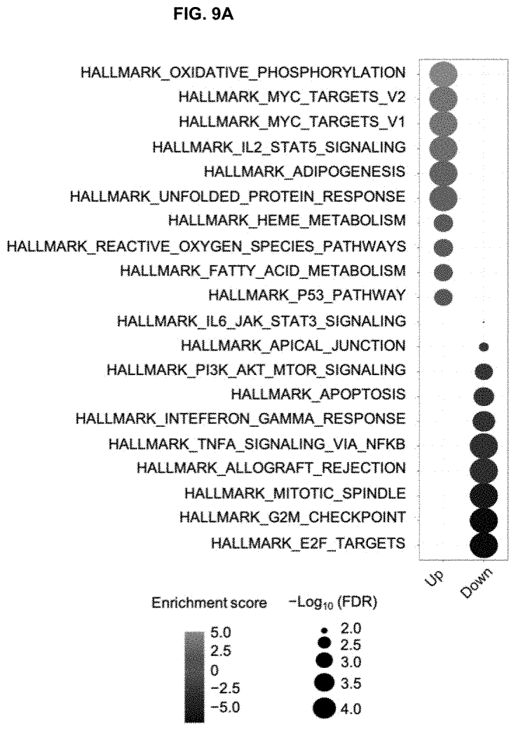

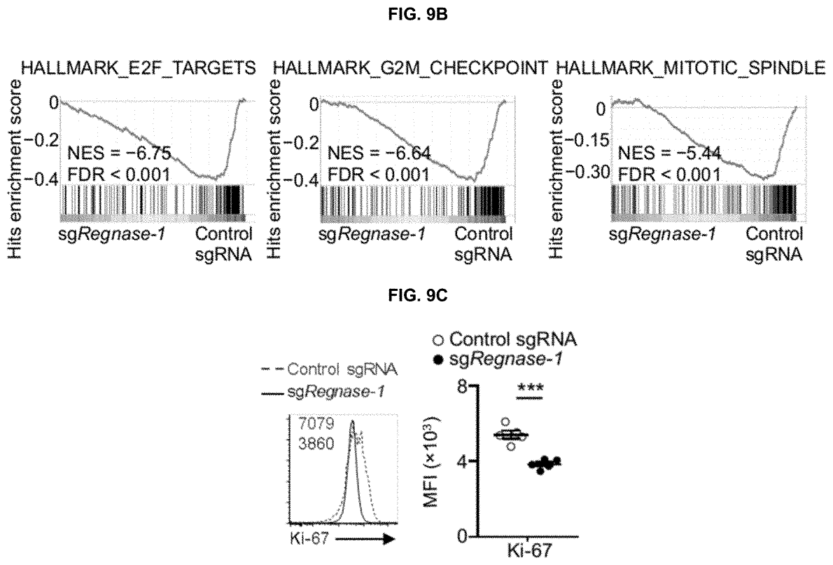

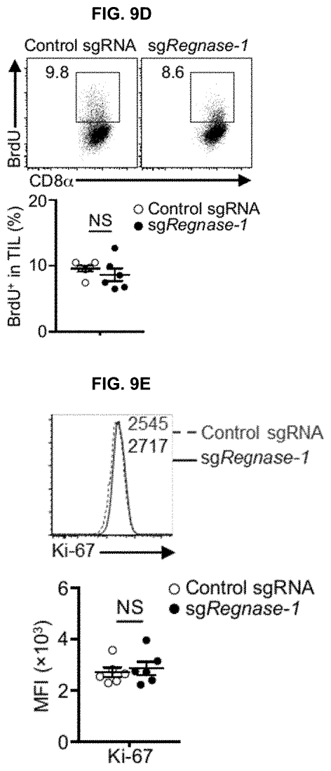

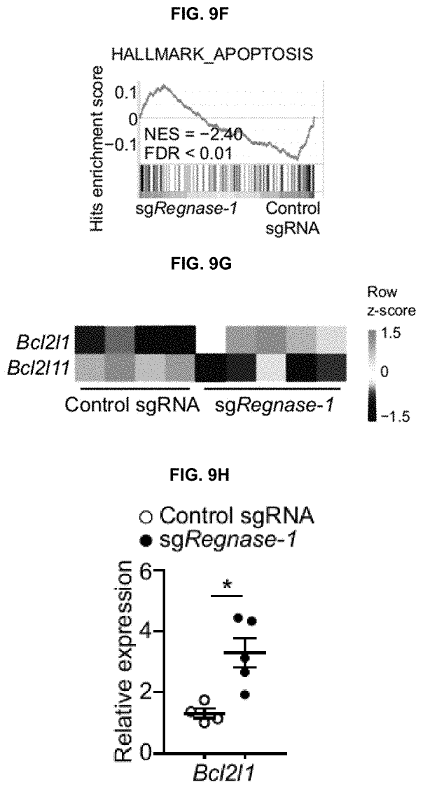

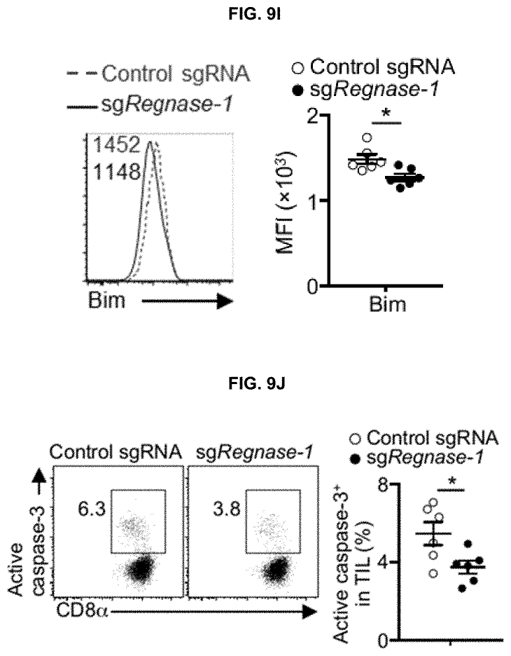

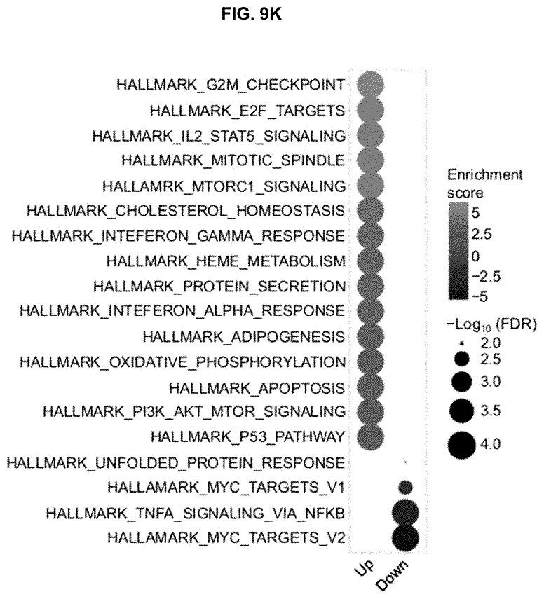

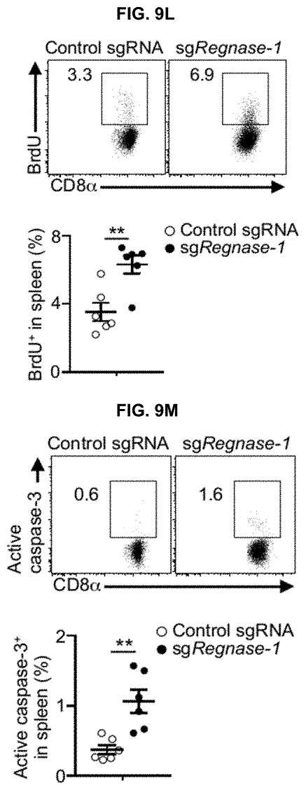

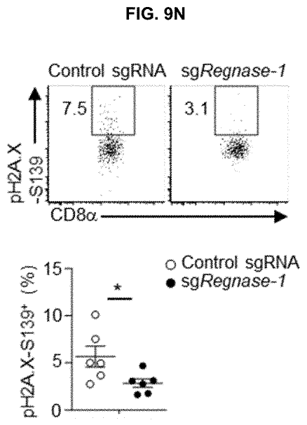

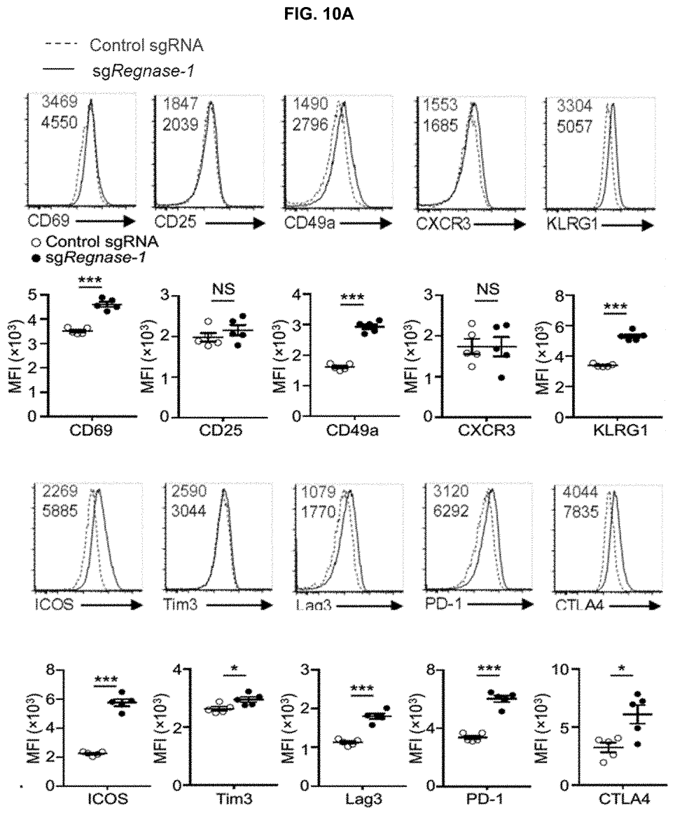

The cellular homeostasis of Regnase-1-null OT-I cells was determined. GSEA using “Hallmark” gene sets revealed that cell cycling-associated hallmarks, including E2F targets, G2M checkpoint and mitotic spindle, were the top three downregulated pathways in tumor-infiltrating Regnase-1-null cells as compared to wild-type cells (FIGS. 9A and 9B). To validate this result, the proliferation of Regnase-1-null OT-I cells was examined by measuring BrdU incorporation and Ki-67 staining. Significantly reduced BrdU incorporation (FIG. 3C) and Ki-67 expression (FIG. 9C) in tumor-infiltrating Regnase-1-null OT-I cells was observed at day 14 after adoptive transfer and, while no difference in BrdU incorporation and Ki-67 expression was observed at day 7 (FIGS. 9D and 9E). The apoptosis hallmark was also one of the top downregulated pathways in tumor-infiltrating Regnase-1-null OT-I cells (FIGS. 9A and 9F). This alteration was associated with increased and reduced expression of anti-apoptotic Bcl2l1 (encodes Bcl-xL) and pro-apoptotic Bcl2l11 (encodes Bim), respectively (FIG. 9G), which was subsequently validated (FIGS. 9H and 9I). In agreement with these results, tumor-infiltrating Regnase-1-null OT-I cells had reduced staining of active caspase-3 at both days 7 and 14 after adoptive transfer (FIGS. 3D and 9J). Tumor-infiltrating Regnase-1-null OT-I cells also had reduced levels of DNA damage, as measured by a specific staining to detect phosphorylation of the histone variant H2A.X at Ser13 9^{61,63} (FIG. 9N). Therefore, tumor-infiltrating Regnase-1-null OT-I cells are less proliferative after initial effector expansion and more importantly, exhibit better survival than wild-type cells in tumors, in line with enhanced survival and the more quiescent state of naive/memory CD8⁺ T cells and long-lived stem cells⁶⁴⁻⁶⁶. In contrast, but consistent with the increased activation signatures described above (FIGS. 7E and 7G), peripheral Regnase-1-null OT-I cells were enriched with cell cycling and apoptosis-associated signatures (FIG. 9K), which was validated by increased BrdU incorporation and active caspase-3 expression in Regnase-1-null OT-I cells in the spleen of tumor-bearing mice (FIGS. 9L and 9M). These results further support TME-specific phenotypes of Regnase-1-null CD8⁺ T cells.

### Example 7

### T Cell in vivo Persistence Assays

It was hypothesized that tumor-infiltrating Regnase-1-null OT-I cells show enhanced in vivo persistence. To this end, WT and Regnase-1-null OT-I cells were isolated from TILs, mixed at 1:1, and transferred them into either inflammation-matched tumor-bearing hosts or na-ve mice (FIG. 3E). In response to tumor antigen stimulation, Regnase-1-null OT-I cells showed much greater accumulation in tumor sites (FIG. 3F). Moreover, in response to homeostatic signals upon transfer into na-ve recipients, Regnase-1-null OT-I cells still showed better persistence in the spleen as compared to wild-type cells (FIG. 3G). Altogether, these extensive bioinformatic analyses and experimental validations indicate that tumor-infiltrating Regnase-1-null CD8⁺ T cells are characterized by in vivo quiescence and cellular survival, and exhibit better in vivo persistence in response to both antigen stimulation and homeostatic signals.

### EXAMPLE 8

### Effector Molecular Expression in Tumor-Infiltrating Regnase-1-Deficient CD8⁺ T cells

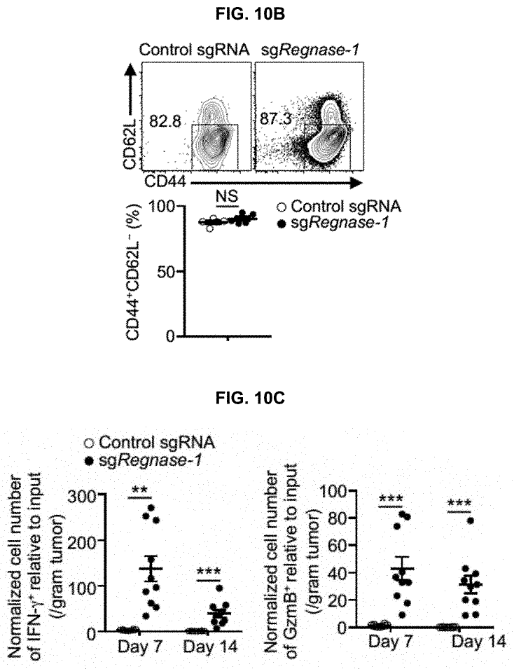

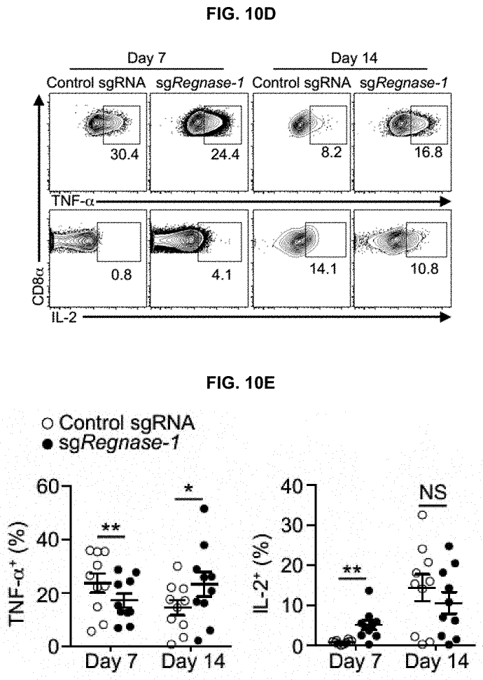

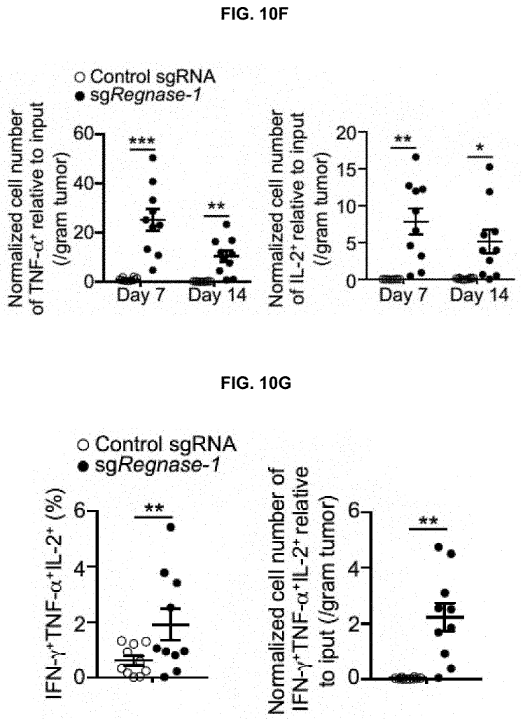

Naïve or memory cells and terminally differentiated effector cells are generally considered as exclusive cell fates^{64,67}. For instance, Tet2-deficient CAR-T cells can adopt a memory cell phenotype with better persistence, but with impaired effector function⁴. Interestingly, despite the acquisition of naive/memory cell-associated gene programs of Regnase-1-null OT-I cells in tumors, they had higher expression of activation-associated markers, including CD69, CD49a, KLRG1, ICOS, Tim3, Lag3, PD-1 and CTLA4, while CD25 and CXCR3 expression was largely normal (FIG. 10A). Also, tumor-infiltrating Regnase-1-null OT-I cells retained an effector surface phenotype (CD44⁺ CD621_,⁻) (FIG. 10B) and, more importantly, contained more IFN-γ− and granzyme B (GzmB)- expressing cells within tumors (except for a minor reduction of GzmB⁺ T cell percentage at day 7) (FIGS. 3H, 3I, 10C). Among IFN-γ⁺and GzmB⁺Regnase-1-null OT-I cells, the expression levels of IFN-γ and GzmB were also increased on a per cell basis (FIG. 3J). Additionally, Regnase-1-null OT-I cells largely preserved the capacity to produce TNF-α (FIGS. 10D and 10E), while there were more TNF-α-producing Regnase-1-null OT-I cells than wild-type cells (FIG. 10F). Moreover, Regnase-1-null OT-I cells produced more IL-2 than wild-type controls at day 7 after adoptive transfer (FIG. 10D and 10E), with increased number of IL-2-producing cells (FIG. 10F). Regnase-1-null OT-I cells also had increased proportion and number of IFN-γ⁺TNF-α⁺IL-2⁺polyfunctional T cells (FIGS. 10G). Thus, although tumor-infiltrating CD8⁺ T cells lacking Regnase-1 acquire better persistence and survival advantage, they retain terminally differentiated effector function.

### Example 9

### scRNA-Seq and flow cytometry analyses of tumor-infiltrating Regnase-1-null OT-I Cells

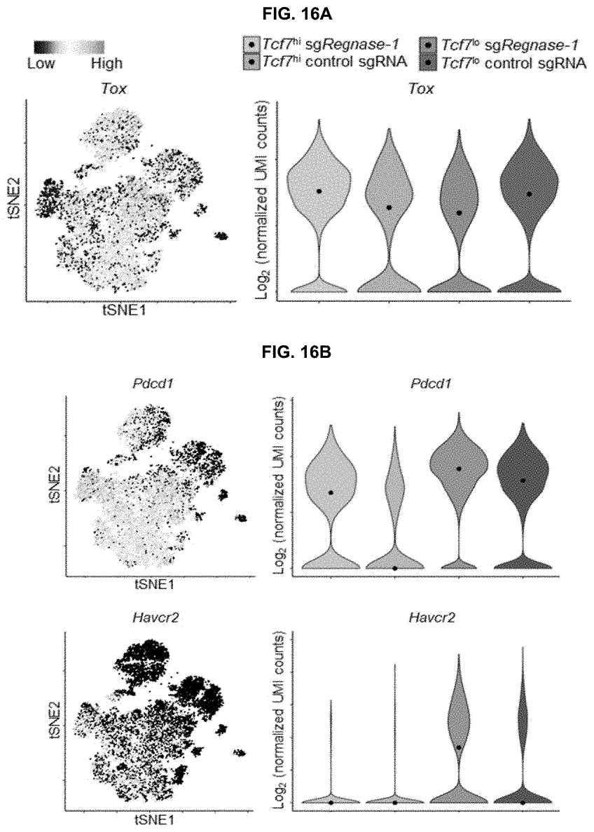

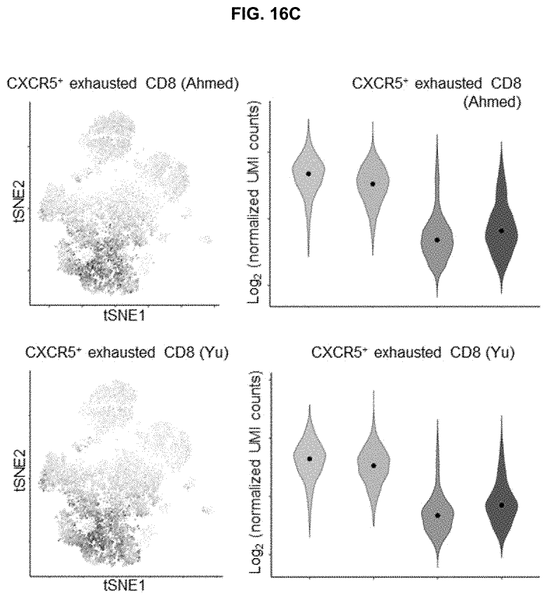

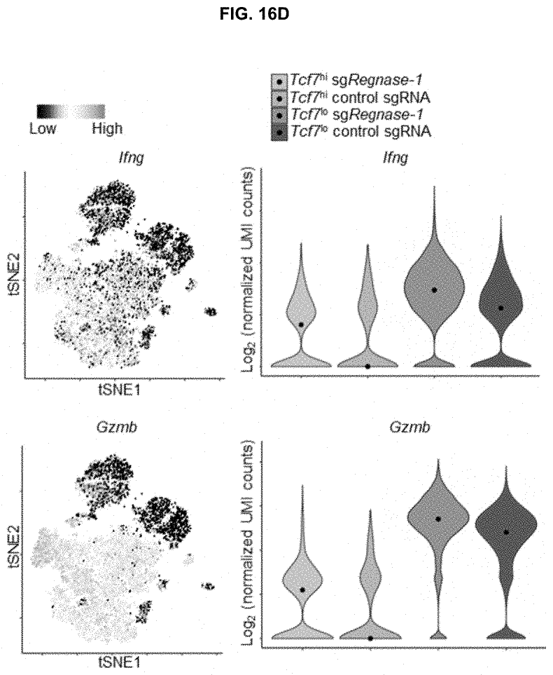

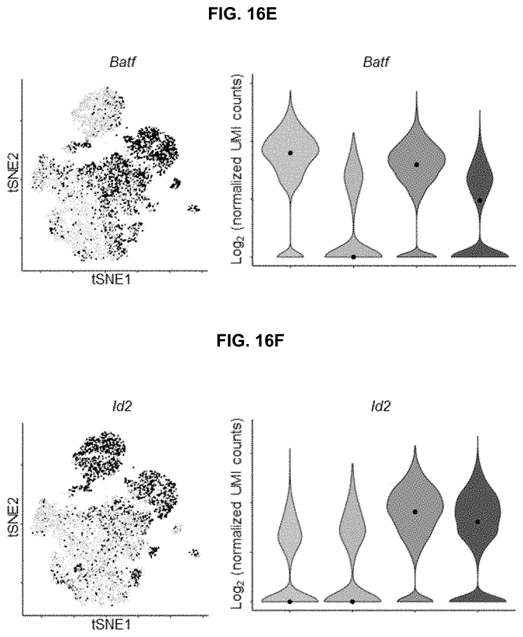

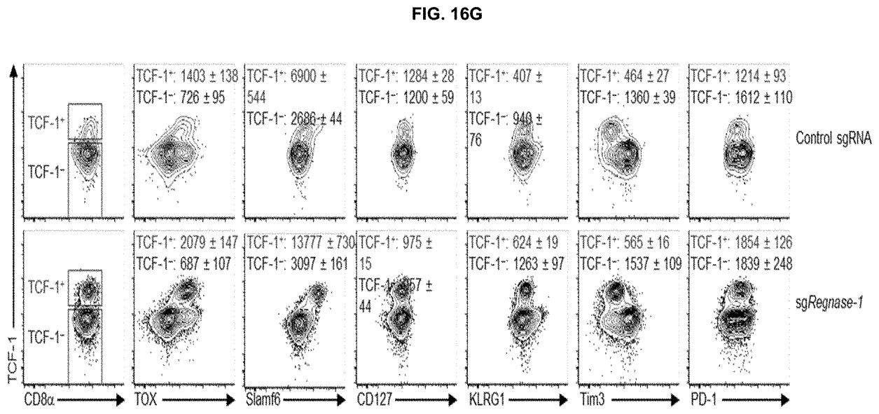

To further determine the effects of Regnase-1 deletion on tumor-specific OT-I cells, single cell RNA-Sequencing (scRNA-Seq)⁶⁸ was used to unbiasedly profile transcriptional programs of TILs isolated from the in vivo dual transfer system at day 7 after adoptive transfer, and identified distinct distribution patterns between Regnase-1-null and wild-type cells (FIG. 3L). Consistent with the increased TCF-1 expression revealed by flow cytometry (FIG. 3K), Regnase-1-null OT-I cells had an increased proportion of Tcf7^{hi} cells (control sgRNA: 19.2%; sgRegnase-1: 31.0%), which also expressed modestly higher levels of Tcf7 than wild-type counterparts (FIGS. 3L and 3M). In chronic infection and tumor-elicited immune responses, the TCF-1⁺memory-like progenitor population has been recently shown to express the transcription factor TOX but have lower expression of immune exhaustion molecules^{7,69-73}. Indeed, it was found that Tcf7^{hi} cells in wild-type and Regnase-1-null TILs expressed Tox (FIG. 16A), but, compared with Tcf7^{lo} cells, had reduced expression of Pdcdl (encodes PD-1) and Havcr2 (encodes Tim3) (FIG. 16B). Moreover, Tcf7^{hi} cells were enriched with memory- or progenitor-like CD8⁺ T cell gene signatures derived from chronic infection^{7,8} (FIG. 16C) and the expression of Slamf6 (a TCF-1-dependent target^{73,74}; FIG. 3M), all of which showed increased expression in Regnase-1-null cells (FIGS. 3M, 16C). In further support of the memory-like phenotype of Tcf7^{hi} cells, they had lower expression of effector genes Ifng and Gzmb than Tcf7^{lo} cells (FIG. 16D). In the absence of Regnase-1, the expression of Ifng and Gzmb was increased in both Tcf7^{hi} and Tcf7^{lo} cells (FIG. 16D), consistent with the increased expression levels of IFN-y and GzmB (FIG. 3J). Moreover, the expression of effector cell-associated transcription factors, including Batf and Id2^{9,37,75,76}, was also increased in Regnase-1-null cells, but with distinct patterns of expression (FIGS. 16E, 16F). Specifically, in wild-type cells, expression of Batf and Id2 was enriched in Tcf7^{lo} cells (FIGS. 16E, 16F). However, in Regnase-1-null cells, while Id2 was still predominantly expressed in Tcf7^{lo} cells, Batf expression was highly expressed by both Tcf7^{hi} and Tcf7^{lo} cells (FIGS. 16E, 16F), indicative of unique transcriptional reprogramming in the absence of Regnase-1. Consistent with the scRNA-Seq analysis, flow cytometry analysis revealed that TCF-1⁺ cells did express TOX, with a modestly higher level observed in the absence of Regnase-1 (FIG. 16G). While CD127/IL-7R was unchanged, TCF-1⁺ cells had high expression levels of Slamf6, which was further elevated in Regnase-1-null cells (FIG. 16G). Conversely, TCF-1⁺ cells had low KLRG1 and Tim3 and intermediate PD-1 expression levels, and these patterns were largely retained in Regnase-1-null cells with modestly enhanced abundance (FIG. 16G). Thus, the TCF-1⁺subset observed in the adoptive transfer system largely resembles the TCF-1⁺memory-like progenitor cells described in chronic infection and other tumor models^{7,69-71,73}. Collectively, these results provide additional support for the dual roles of Regnase-1 in coordinating T cell effector function and persistence in antitumor immunity, by increasing the proportion and signature molecules of memory/progenitor-like CD8⁺ T cells while retaining the robust effector function.

For scRNA-Seq library preparation, control sgRNA- and sgRegnase-1-transduced OT-I cells were sorted on an iCyt Reflection cell sorter from TILs pooled from the in vivo dual transfer hosts (6-8 mice per sample) at day 7 after adoptive transfer into tumor-bearing mice. The cells were counted and examined for viability using a Luna Dual Florescence Cell Counter (Logos Biosystems). All samples were spun down at 2,000 rpm for 5 min. The supernatant was removed, and cells were re-suspended in 100 μl of 1 ×PBS (Thermo Fisher Scientific)+0.04% BSA (Amresco). The cells were then counted and examined for viability using a Luna Dual Florescence Cell Counter (Logos Biosystems). Cell counts were about 1×10⁶ cells per milliliter and viability was above 98%. Single-cell suspensions were loaded onto the Chromium Controller according to their respective cell counts to generate 6,000 single cell GEMs (gel beads in emulsion) per sample. Each sample was loaded into a separate channel. Libraries were prepared using the Chromium Single Cell 3′ v2 Library and Gel Bead Kit (10x Genomics). The cDNA content of each sample after cDNA amplification of 12 cycles was quantified and quality checked using a High-Sensitivity DNA chip with a 2100 Bioanalyzer (Agilent Technologies) to determine the number of PCR amplification cycles to yield sufficient library for sequencing. After library quantification and quality check by DNA 1000 chip (Agilent Technologies), samples were diluted to 3.5 nM for loading onto the HiSeq 4000 (Illumina) with a 2×75 paired-end kit using the following read length: 26 bp Readl, 8 bp i7 Index, and 98 bp Read2. An average of 400,000,000 reads per sample was obtained (approximately 80,000 reads per cell).

For Alignment, barcode assignment and unique molecular identifier (UMI) counting, the Cell Ranger 1.3 Single-Cell software suite (10× Genomics) was implemented to process the raw sequencing data from the Illumina HiSeq run. This pipeline performed demultiplexing, alignment (using the mouse genome mm10 from ENSEMBL GRCm38), and barcode processing to generate gene-cell matrices used for downstream analysis. Specifically, data from two control sgRNA and two sgRegnase-1-transduced TIL OT-I cell samples were combined into one data set for consistent filtering, and UMIs mapped to genes encoding ribosomal proteins were removed. Cells with low UMI counts (potentially dead cells with broken membranes) or high UMI counts (potentially two or more cells in a single droplet) were filtered. A small fraction of outlier cells (888) was further removed because of their low transcriptome diversity (meaning that fewer genes were detected than in other cells with a comparable number of captured UMIs). A total of 13,879 cells (control sgRNA-transduced, 6,811; sgRegnase-1-transduced, 7,068) were captured, with an average of 11,040 messenger RNA molecules (UMIs, median: 9,391; range: 2,928-44,330). The expression level of each gene was normalized to 100,000 UMIs per cell and log-transformed them by adding 0.5 to the expression matrix.

For data visualization, underlying cell variations derived from control sgRNA- and sgRegnase-1-transduced TIL OT-I cell single-cell gene expression were visualized in a two-dimensional projection by t-distributed stochastic neighbour embedding (tSNE). Expression of individual genes or pathway scores was color-coded (from low to high, blue-red) for each cell on tSNE plots. To visualize Tcf7-expressing cells, Tcf7^{hi} cells were defined as cells with the highest third quantile of Tcf7 expression (with log₂ gene expression intensity =2.910317 as threshold) among all cells.

### Example 10

### Identification of Immune Regulators and OXPHOS Metabolic Pathway using Genome-Scale CRISPR-Cas9 Screening

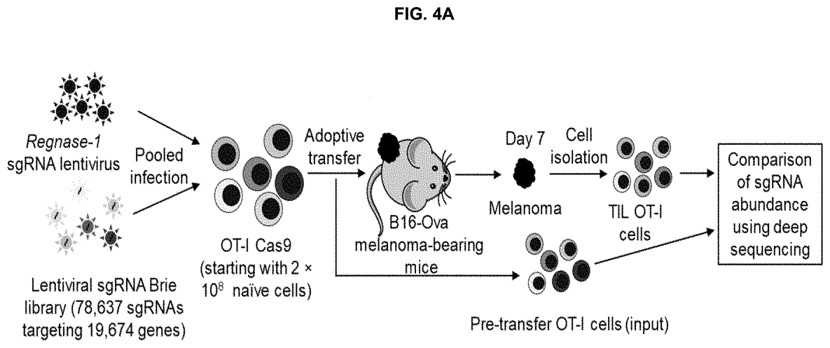

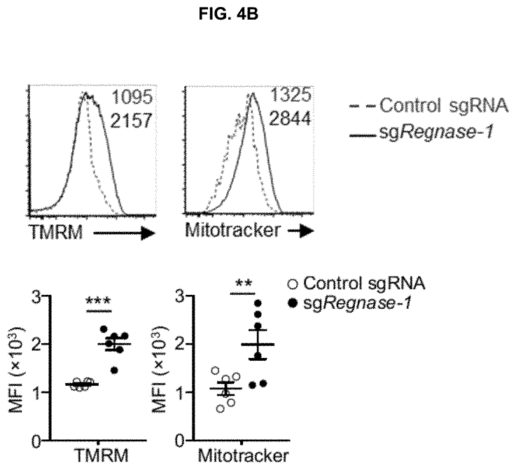

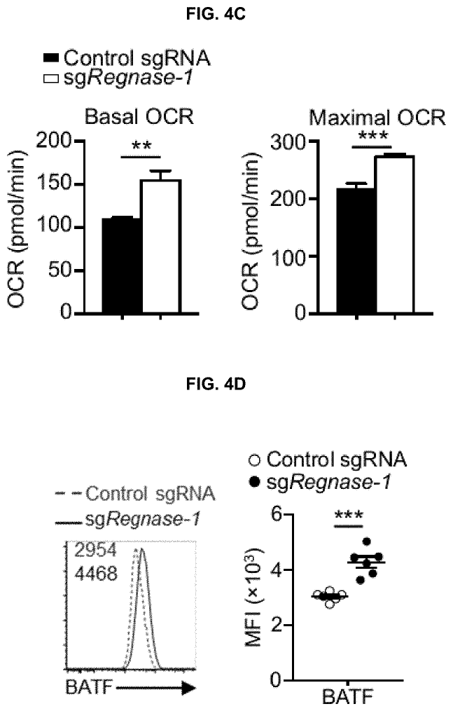

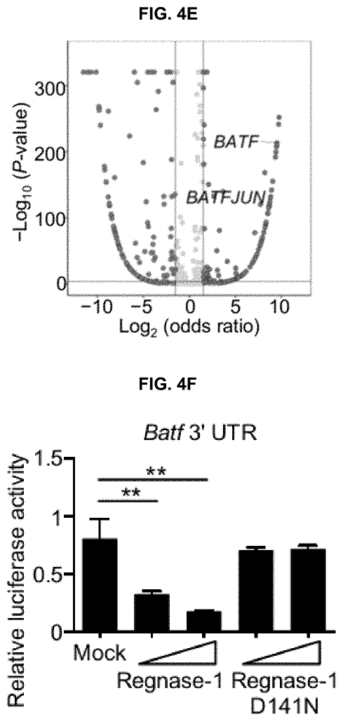

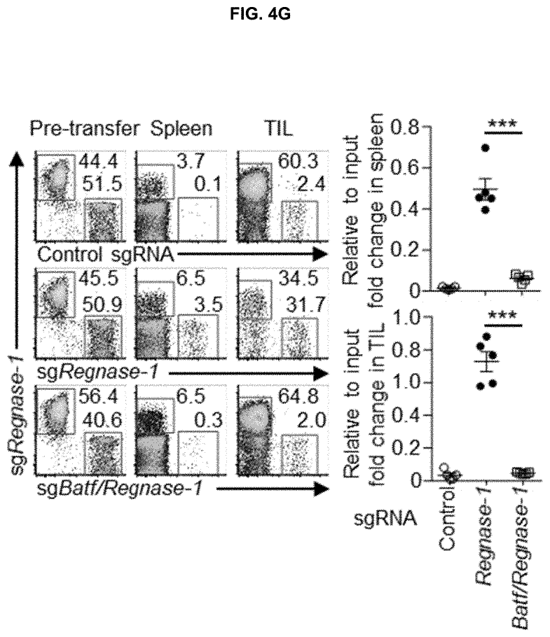

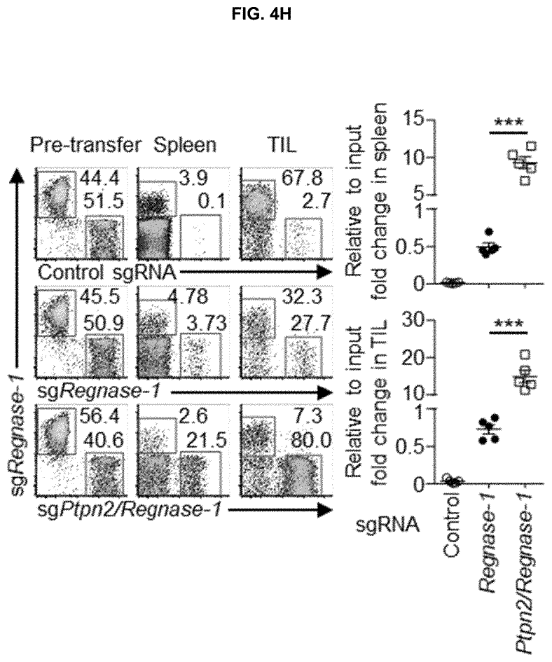

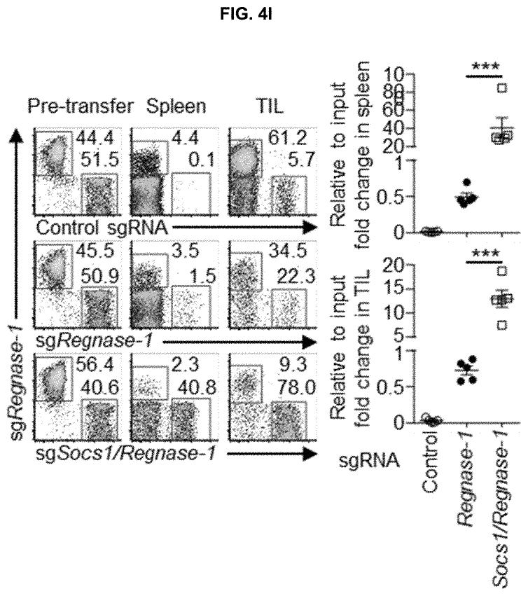

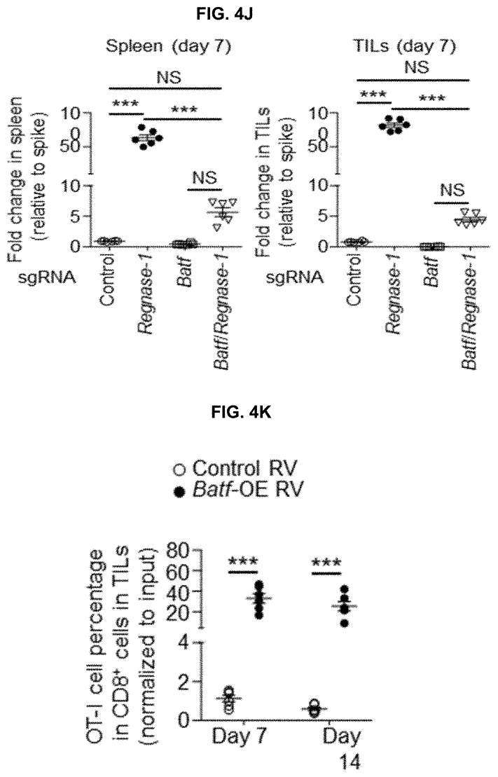

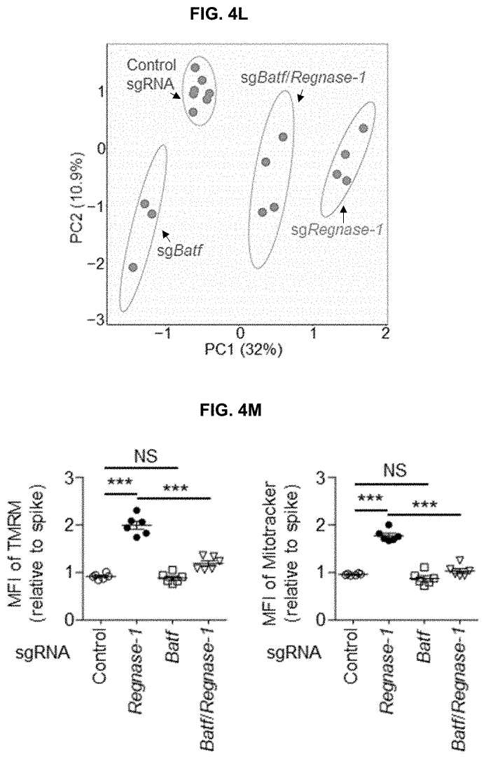

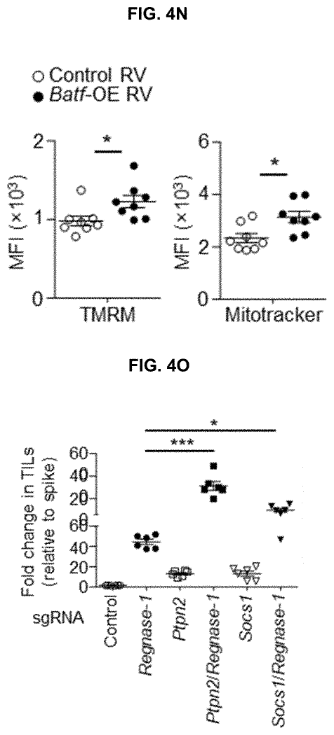

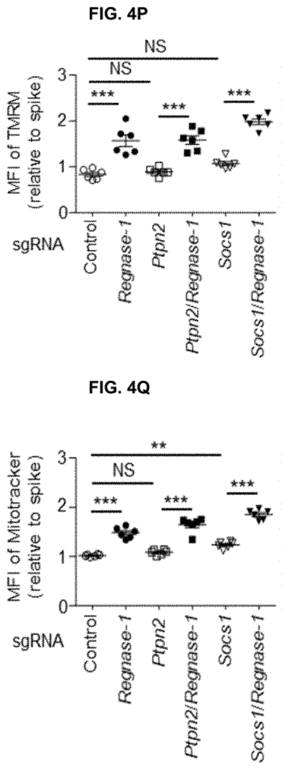

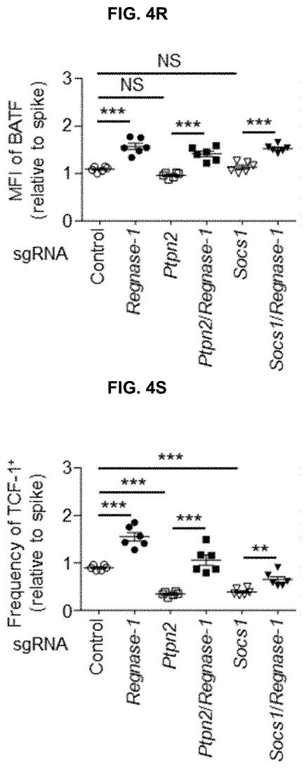

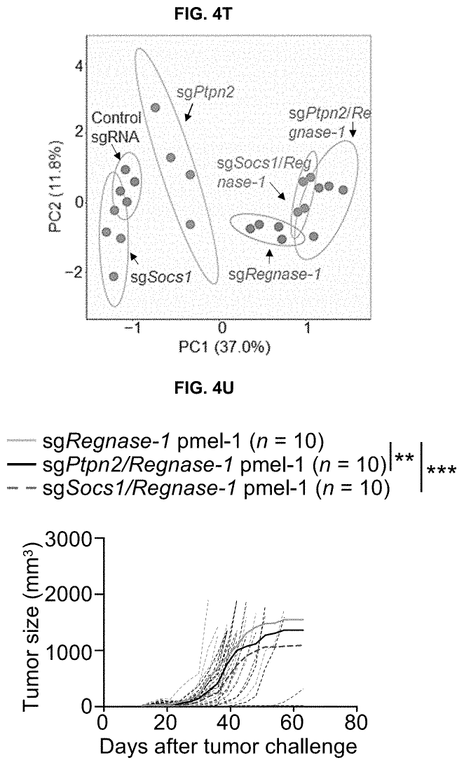

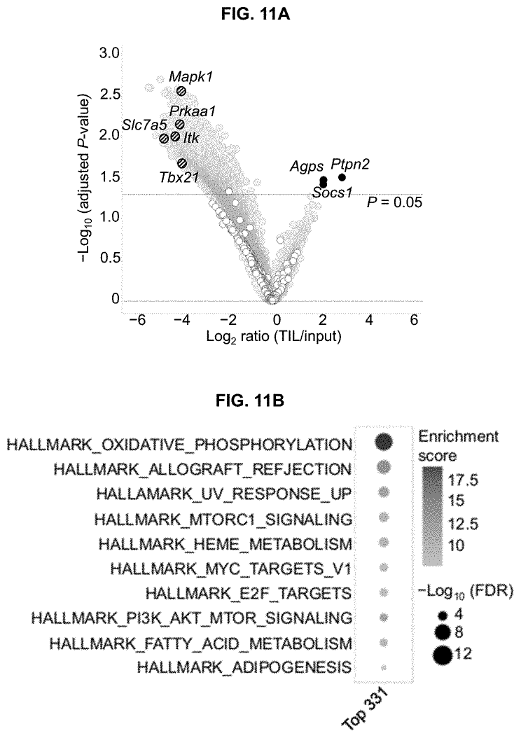

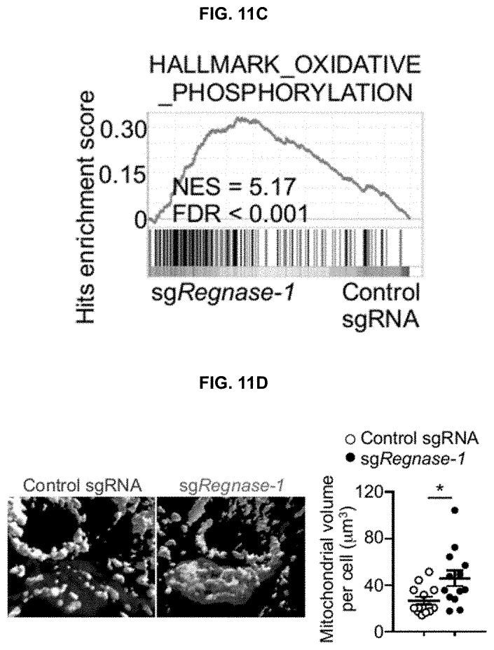

To probe the molecular pathways underlying Regnase-1-mediated protective immunity in tumor-specific CD8⁺ T cells, a secondary in vivo genome-scale CRISPR-Cas9 mutagenesis screening was performed using Regnase-1-deficient OT-I cells in the melanoma B16-Ova model (FIG. 4A). Given that Regnase-1 has RNase activity and inhibits target gene expression^{15,16}, the enhanced immune responses of Regnase-1-null OT-I cells were expected to be suppressed by deleting functionally important targets of Regnase-1. Specifically, OT-I T cells were transduced with sgRegnase-1 and Brie lentiviral genome-scale sgRNA library that consists of 78,637 sgRNAs targeting 19,674 genes⁷⁷, and after selection of dual-transduced cells (based on puromycin resistance and Ametrine⁺ cell sorting) and adoptive transfer into tumor-bearing host, isolated OT-I cells for deep sequencing. Candidate genes were ranked based on the average enrichment of their sgRNAs (4 sgRNAs per gene) in tumor-infiltrating OT-I cells relative to input (log₂ ratio (TIL/input); adjusted P<0.05) (FIG. 11A and Table 1). Using a stringent cutoff (by less than −3.5 log₂ ratio or >10 fold reduction (TIL/input); adjusted P<0.05), a total of 331 genes were identified that were strongly depleted in the screening, including known regulators of T cell expansion and effector differentiation such as Slc7a5 (encodes LAT1)⁷⁸ (log₂ ratio (TIL/input) =−4.75), Itk⁷⁹ (log₂ ratio (TIL/input)=−4.29), Prkaal (encodes AMPKα1)⁸⁰ (log₂ ratio (TIL/input) =−4.09), Mapk1 (encodes Erk2)⁸¹ (10g2 ratio (TIL/input)=−4.03) and Tbx21 (encodes T-bet)⁸² (log₂ ratio (TIL/input)=−3.95) (FIG. 11A). Functional enrichment of the top-ranking depleted genes was performed next, which revealed that oxidative phosphorylation (OXPHOS) hallmark was the top-ranked pathway (FIG. 11B), suggesting a possible role for oxidative metabolism in supporting the excessive accumulation of Regnase-1-null OT-I cells in tumor immunity. In support with this notion, increased OXPHOS metabolism has been shown to correlate with improved fitness of effector T cells and their antitumor activityl^{3,83,84}. Among the gene sets enriched in tumor-infiltrating Regnase-1-null OT-I cells relative to wild-type cells (FIG. 9A), OXPHOS was the top ranking one (FIG. 11C). Therefore, mitochondrial profiles and oxidative metabolism were measured. Oxygen consumption rates (OCR, indicative of OXPHOS activity) were measured in XF media under basal conditions and in response to 1 μM oligomycin, 1.5 μM fluoro-carbonyl cyanide phenylhydrazone (FCCP) and 500 nM rotenone using an XF96 Extracellular Flux Analyzer (EFA) (Seahorse Bioscience). Regnase-1-null OT-I cells had increased mitochondrial fitness, as indicated by increased mitochondrial mass, membrane potential (FIG. 4B) and volume (FIG. 11D). They also had significantly higher basal and maximal OCR) (FIG. 4C), indicating enhanced oxidative metabolism.

For the genome-scale sgRNA Brie library CRISPR-Cas9 mutagenesis screening, the following methods were used.

In vivo screening: Lentivirus was produced by co-transfecting HEK293T cells with lentiviral genome-scale Brie library plasmids with the puromycin resistant gene⁷⁷, psPAX2 and pCAG4-Eco. At 48 h after transfection, virus was harvested and froze at −80° C. Two hundred million Cas9-expressing OT-I cells were isolated from 12 Cas9-OT-I mice and co-transduced with Brie sgRNA library and sgRegnase-1-Ametrine. After viral transduction, cells were cultured with human IL-2 (20 IU/ml; PeproTech), mouse IL-7 (25 ng/ml; PeproTech) and IL-15 (12.5 ng/ml; PeproTech) for 2 days. Brie sgRNA library-transduced cells were then selected by culture with 4 μg/ml puromycin in the presence of the abovementioned cytokines for another 3 days. Following puromycin selection, Ametrine⁺ cells were sorted using a Reflection sorter (i-Cyt) to select for cells co-transduced with sgRenase-1 and Brie library sgRNAs, and an aliquot of 10×10⁶ transduced OT-I cells was saved as input (-120× cells coverage per sgRNA). The majority of the co-transduced OT-I cells (5×10⁶ cells per recipient) were then i.v. transferred into mice at day 14 after B16-Ova melanoma engraftment. Twenty recipients were randomly divided into 2 groups as biological replicates. At 7 days after adoptive transfer, transferred Ametrine⁺OT-I cells were recovered from the tumor pooled from 10 recipients per sample using a Reflection sorter (i-Cyt). On average, 3×10⁶ OT-I cells per sample (˜40× cell coverage per sgRNA) were recovered. DNA extraction and sequencing library preparation were as described in Example 1.

Data processing: For data analysis, FastQ files obtained after sequencing were demultiplexed using the Hi Seq Analysis software (Illumina). sgRegnase-1 (GGAGTGGAAACGCTTCATCG; (SEQ ID NO: 2)) reads were removed, and single-end reads were trimmed and quality-filtered using the CLC Genomics Workbench vii (Qiagen) and matched against sgRNA sequences from the genome-scale sgRNA Brie library. Read counts for sgRNAs were normalized against total read counts across all samples. For each sgRNA, the fold change (log2 ratio) for enrichment was calculated between each of the biological replicates and the input experiment. Gene ranking was based on the average enrichment among replicates in representation of 4 individual corresponding sgRNAs in the genome-scale sgRNA Brie library. The gene level false discovery rate (FDR) adjusted P-value was calculated among multiple sgRNAs of each gene, using a paired two-tailed t-test between log₂ transformed average normalized read counts of tumor samples and those of input sample, and a value of less than 0.05 was considered to be statistically significant. Identified candidate genes with log2 ratio (TIL/input) >1 are presented in Table 1.

TABLE 1
Brie library CRISPR-Cas9 mutagenesis screening results

| ID | Symbol | log₂R_B_avg | log₂R_C_avg | log₂R_avg | Rank_avg | Flag_avg | FDR |
|---|---|---|---|---|---|---|---|
| 19255 | Ptpn2 | 3.03103694 | 3.02918452 | 3.03011073 | 1 | 1 | 0.03074379 |
| 12703 | Socs1 | 2.52286933 | 1.91519814 | 2.21903373 | 2 | 1 | 0.03771928 |
| 228061 | Agps | 2.3661194 | 2.06106624 | 2.21359282 | 3 | 1 | 0.03416105 |
| 64602 | Ireb2 | 1.61204469 | 2.33695115 | 1.97449792 | 4 | 1 | 0.06996736 |
| 53611 | Vti1a | 1.86939233 | 1.78635701 | 1.82787467 | 5 | 1 | 0.09730926 |
| 217864 | Rcor1 | 1.85655273 | 1.74145244 | 1.79900259 | 6 | 1 | 0.0894387 |
| 73481 | 1700074P13Rik | 1.83755799 | 1.62905795 | 1.73330797 | 7 | 1 | 0.06609545 |
| 72129 | Pex13 | 1.95120807 | 1.45465932 | 1.7029337 | 8 | 1 | 0.09887023 |
| 381305 | Rc3h1 | 1.40911805 | 1.92142968 | 1.66527387 | 9 | 1 | 0.09909689 |
| 74245 | Ctbs | 1.88845492 | 1.42972741 | 1.65909116 | 10 | 1 | 0.05287707 |
| 106869 | Tnfaip8 | 1.71532245 | 1.54472754 | 1.630025 | 11 | 0 | 0.09843569 |
| 73660 | Cabp4 | 1.45873412 | 1.76204659 | 1.61039035 | 12 | 0 | 0.10567293 |
| 114604 | Prdm15 | 1.33459848 | 1.82703175 | 1.58081512 | 13 | 0 | 0.0838152 |
| 171508 | Creld1 | 1.62395687 | 1.5350195 | 1.57948818 | 14 | 0 | 0.09368323 |
| 74315 | Rnf145 | 1.49877812 | 1.59969823 | 1.54923818 | 15 | 0 | 0.07332863 |
| 14590 | Ggh | 1.65670065 | 1.39743515 | 1.5270679 | 16 | 0 | 0.13589762 |
| 19051 | Ppp1r17 | 1.62840681 | 1.39779346 | 1.51310013 | 17 | 0 | 0.12254913 |
| 17224 | Mcpt1 | 1.37367256 | 1.630174 | 1.50192328 | 18 | 0 | 0.13821568 |
| 266690 | Cyb5r4 | 1.3572997 | 1.59893185 | 1.47811577 | 19 | 0 | 0.13447517 |
| 71726 | Smug1 | 1.5026502 | 1.41804068 | 1.46034544 | 20 | 0 | 0.12097533 |
| 629203 | Sult2a3 | 1.53636928 | 1.32653562 | 1.43145245 | 21 | 0 | 0.10305758 |
| 21833 | Thra | 1.43072319 | 1.39081573 | 1.41076946 | 22 | 0 | 0.14204162 |
| 67921 | Ube2f | 1.49084237 | 1.30926107 | 1.40005172 | 23 | 0 | 0.14858738 |
| 258238 | Olfr417 | 1.43977446 | 1.35008627 | 1.39493037 | 24 | 0 | 0.15012533 |
| 258977 | Olfr1273-ps | 1.23353661 | 1.52437603 | 1.37895632 | 25 | 0 | 0.17499336 |
| 17537 | Meis3 | 1.44567538 | 1.30409252 | 1.37488395 | 26 | 0 | 0.1302195 |
| 17448 | Mdh2 | 1.30097159 | 1.43134239 | 1.36615699 | 27 | 0 | 0.11483854 |
| 83553 | Tktl1 | 1.4360966 | 1.28791599 | 1.3620063 | 28 | 0 | 0.12767619 |
| 12626 | Cetn3 | 1.25505016 | 1.39902074 | 1.32703545 | 29 | 0 | 0.12830047 |
| 108857 | Ankhd1 | 1.21177106 | 1.43606478 | 1.32391792 | 30 | 0 | 0.16797524 |
| 18214 | Ddr2 | 1.29435522 | 1.31365101 | 1.30400311 | 31 | 0 | 0.13323973 |
| 74365 | Lonrf3 | 1.34669339 | 1.25584666 | 1.30127002 | 32 | 0 | 0.16294965 |
| 234577 | Cpne2 | 1.11993149 | 1.47584619 | 1.29788884 | 33 | 0 | 0.12901258 |
| 258159 | Olfr1331 | 1.37014924 | 1.21901921 | 1.29458422 | 34 | 0 | 0.1226376 |
| 381101 | Dnph1 | 1.38491612 | 1.17420704 | 1.27956158 | 35 | 0 | 0.20233129 |
| 56375 | B4galt4 | 1.45453494 | 1.0816909 | 1.26811292 | 36 | 0 | 0.14483496 |
| 231098 | Dnajc5g | 1.26529554 | 1.25267673 | 1.25898613 | 37 | 0 | 0.14616519 |
| 20681 | Sox8 | 1.23180433 | 1.27755354 | 1.25467894 | 38 | 0 | 0.17046512 |
| 68396 | Nat8 | 1.3205451 | 1.18055343 | 1.25054927 | 39 | 0 | 0.16931487 |
| 12417 | Cbx3 | 1.25464055 | 1.2456972 | 1.25016887 | 40 | 0 | 0.16362677 |
| 17681 | Msc | 1.14559315 | 1.33318201 | 1.23938758 | 41 | 0 | 0.15065439 |
| 66598 | 3110001I22Rik | 1.20860211 | 1.25541445 | 1.23200828 | 42 | 0 | 0.19770332 |
| 329421 | Myo3b | 1.00022792 | 1.4461835 | 1.22320571 | 43 | 0 | 0.11593875 |
| 232813 | Shisa7 | 1.37239391 | 1.07397853 | 1.22318622 | 44 | 0 | 0.30035235 |
| 21951 | Tnks | 1.1330844 | 1.28120008 | 1.20714224 | 45 | 0 | 0.16526672 |
| 18416 | Otc | 1.26408708 | 1.13542511 | 1.19975609 | 46 | 0 | 0.14019333 |
| 218066 | Olfr11 | 1.18323184 | 1.19603223 | 1.18963204 | 47 | 0 | 0.17348075 |
| 78600 | Pde6h | 0.98143242 | 1.39433648 | 1.18788445 | 48 | 0 | 0.13883771 |
| 72748 | Hdhd3 | 1.33344267 | 1.02469287 | 1.17906777 | 49 | 0 | 0.22056486 |
| 15248 | Hic1 | 1.01260398 | 1.32168842 | 1.1671462 | 50 | 0 | 0.11389018 |
| 16576 | Kif7 | 1.33447886 | 0.99297093 | 1.1637249 | 51 | 0 | 0.1687016 |
| 50934 | Slc7a8 | 1.21735282 | 1.08498001 | 1.15116641 | 52 | 0 | 0.24675046 |
| 74178 | Stk40 | 1.18633507 | 1.11330582 | 1.14982045 | 53 | 0 | 0.12883616 |
| 224647 | D17Wsu92e | 1.18292766 | 1.09612926 | 1.13952846 | 54 | 0 | 0.12800516 |
| 17168 | Nprl3 | 1.63596781 | 0.60503004 | 1.12049892 | 55 | 0 | 0.13610581 |
| 17684 | Cited2 | 1.18782682 | 1.01929634 | 1.10356158 | 56 | 0 | 0.13543798 |
| 81018 | Rnf114 | 0.97503528 | 1.20986689 | 1.09245108 | 57 | 0 | 0.18776651 |
| 216961 | Coro6 | 1.10233206 | 1.07969782 | 1.09101494 | 58 | 0 | 0.16732777 |
| 12395 | Runx1t1 | 0.9121264 | 1.26381302 | 1.08796971 | 59 | 0 | 0.1861355 |
| 73542 | Tssk5 | 1.11901358 | 1.03928858 | 1.07915108 | 60 | 0 | 0.25320596 |
| 114713 | Rasa2 | 0.95787278 | 1.18588326 | 1.07187802 | 61 | 0 | 0.18835655 |
| 14025 | Bcl11a | 1.10895794 | 1.01764532 | 1.06330163 | 62 | 0 | 0.14133973 |
| 100041488 | Gm11937 | 0.78280966 | 1.33224531 | 1.05752749 | 63 | 0 | 0.21985822 |
| 23829 | C1ql1 | 0.84856001 | 1.24144288 | 1.04500145 | 64 | 0 | 0.16564172 |
| 60534 | Fancg | 1.32275719 | 0.76613433 | 1.04444576 | 65 | 0 | 0.23150097 |
| 19246 | Ptpn1 | 1.09330431 | 0.98820958 | 1.04075695 | 66 | 0 | 0.13792429 |
| 20822 | Trove2 | 1.13677884 | 0.9292867 | 1.03303277 | 67 | 0 | 0.20221657 |
| 12349 | Car2 | 0.95841521 | 1.10573626 | 1.03207573 | 68 | 0 | 0.1776024 |
| 433256 | Acsl5 | 1.1767148 | 0.88159349 | 1.02915414 | 69 | 0 | 0.22402518 |
| 66641 | Sike1 | 1.03689296 | 1.01735978 | 1.02712637 | 70 | 0 | 0.22946066 |
| 83456 | Mov10l1 | 1.14324463 | 0.90902104 | 1.02613283 | 71 | 0 | 0.36017167 |
| 22157 | Tulp1 | 0.9667526 | 1.08349213 | 1.02512236 | 72 | 0 | 0.2234767 |
| 12367 | Casp3 | 1.33981505 | 0.70711521 | 1.02346513 | 73 | 0 | 0.20990722 |
| 67290 | 3110040N11Rik | 1.26875939 | 0.77591139 | 1.02233539 | 74 | 0 | 0.18352335 |
| 258564 | Olfr1015 | 1.09141848 | 0.95157856 | 1.02149852 | 75 | 0 | 0.23700427 |
| 72361 | Ces2g | 1.08807329 | 0.91577384 | 1.00192357 | 76 | 0 | 0.13846138 |

### Example 11

### Identification of a functional target of Regnase-1 in tumor immunity

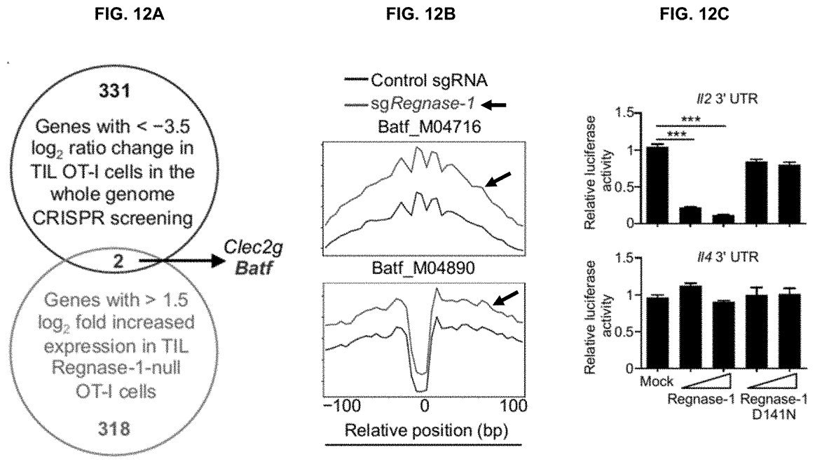

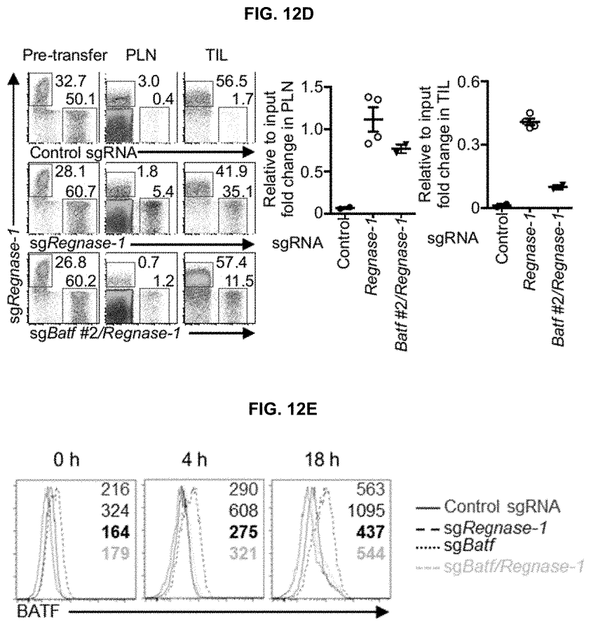

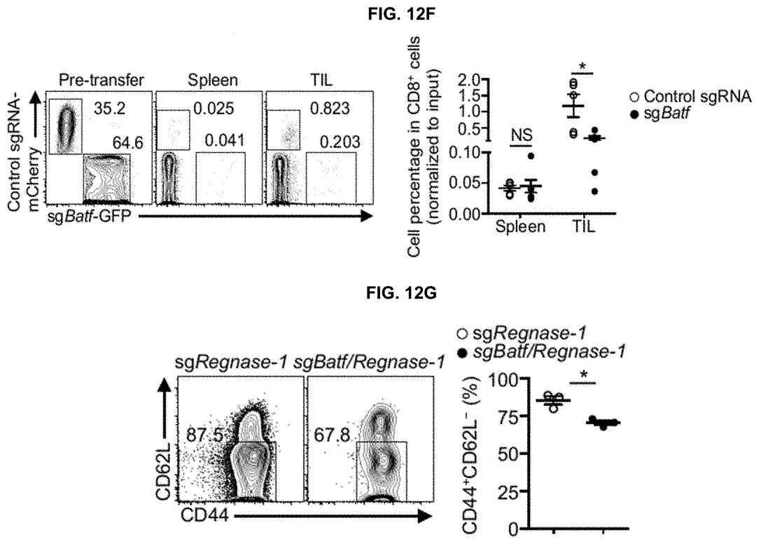

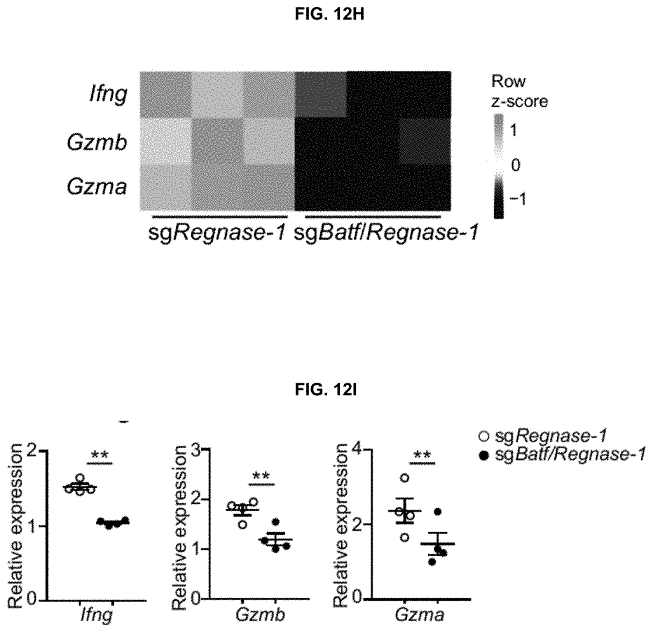

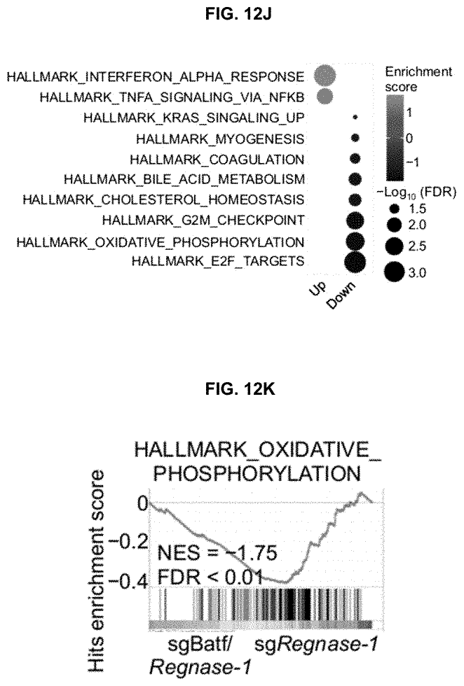

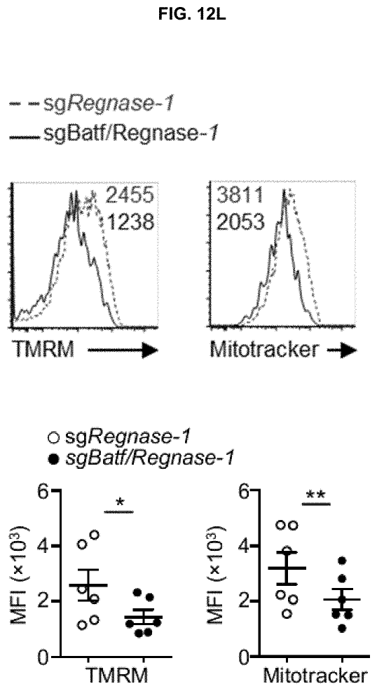

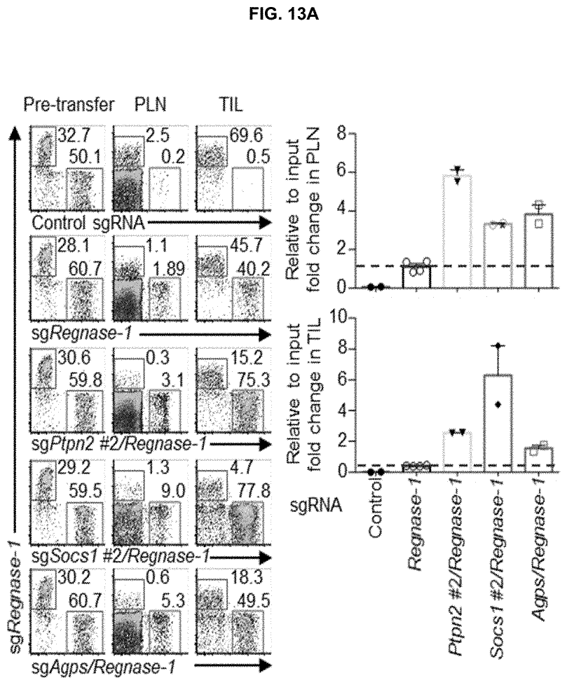

Studies were carried out to probe the functional target whose deletion could suppress the excessive accumulation of Regnase-1-null OT-I cells in tumor immunity. Given the role of Regnase-1 in inhibiting gene expression^{15,16}, two criteria were applied to narrow down the candidates: such candidates should be upregulated in the absence of Regnase-1 in the RNA-Seq analysis, and depleted in TILs in the genome-scale CRISPR-Cas9 screening. Overlaying the top depleted genes (by less than −3.5 log₂ (TIL/input) fold change; adjusted P<0.05) of the genome-scale CRISPR-Cas9 screening with the top upregulated genes (by greater than 1.5 log₂ fold change; P<0.05) in RNA-Seq analysis of Regnase-1-null OT-I cells revealed 2 common candidates, including the transcription factor Batf (FIG. 12A). BATF is a pioneer factor that controls chromatin accessibility allowing subsequent binding by other transcription factors, and is important for T cell differentiation and effector function^{9,75}. Flow cytometry analysis revealed significantly increased BATF expression in Regnase-1-null OT-I cells (FIG. 4D), in agreement with the scRNA-Seq data (FIG. 16E). Consistent with the increased BATF expression, BATF binding motifs were highly enriched in open chromatin regions in Regnase-1-null cells (FIGS. 4E 12B, and 8E). Moreover, the published BATF-binding targets identified from in vitro activated CD8⁺ T cells⁹ were superimposed with the differentially accessible peaks altered in Regnase-1-null cells in the ATAC-Seq data, based on the statistical bioinformatic method used successfully in the context of limited cell numbers^{50,60}. It was found that BATF binding motifs were significantly enriched in Regnase-1-null but not wild-type cells (FIG. 17A). As Regnase-1 has RNase activity and mediates mRNA degradation via targeting the 3′ UTR^{15,16}, whether Batf mRNA is regulated by Regnase-1 was determined by following a published procedure¹⁶. Consistent with a published report¹⁶, the 3′ UTR of Il2, but not Il4, was sufficient to confer destabilization of luciferase upon Regnase-1 overexpression, and this regulation was lost when using a nuclease-inactive form of Regnase-1 (Regnase-1 D141N) (FIG. 12C). Notably, the 3′ UTR of Batf was dose-dependently inhibited by Regnase-1, but not Regnase-1 D141N (FIG. 4F), indicating that BATF is a novel Regnase-1 target. To directly examine the contribution of aberrant BATF expression to the excessive accumulation of Regnase-1-null OT-I cells, the accumulation of Regnase-1-null OT-I cells transduced with sgRNA targeting Batf was measured. Remarkably, co-deletion of BATF drastically reduced the accumulation of Regnase-1-null OT-I cells in both the periphery and tumor (FIGS. 4G and 12D) at days 5 (FIG. 17B) and 7 (FIG. 4J) after adoptive transfer, with the loss of BATF expression validated by flow cytometry and immunoblot analyses (FIGS. 12E, 17C and 17D) . Deletion of BATF itself was also found to impair the accumulation of OT-I cells in the tumor, albeit not in the periphery (FIG. 12F). Therefore, Regnase-1 targets BATF to impair accumulation of CD8⁺ T cells in tumor immunity. Consistent with the context-dependent roles of BATF in mediating antiviral CD8⁺ T cell effector responses^{9,85}, BATF/Regnase-1-null OT-I cells had reduced effector surface phenotypes (FIG. 12G) and expression of effector molecules, including Ifng, Gzmb and Gzma, compared with Regnase-1-null cells (FIGS. 12H and 12I). Transcriptome analysis of BATF/Regnase-1-null and Regnase-1-null OT-I cells were performed next, and GSEA was performed using “Hallmark” gene sets. Compared with Regnase-1-null cells, BATF/Regnase-1-null cells had downregulation of OXPHOS and cell cycle-associated hallmarks (FIGS. 12J and 12K), consistent with the role of BATF in mediating Regnase-1-null effector cell expansion and accumulation. In support of a role of BATF in promoting oxidative metabolism of Regnase-1-null OT-I cells, mitochondrial profiles, including mitochondrial mass and membrane potential, were dampened in BATF/Regnase-1-null OT-I cells, compared with Regnase-1-null cells (FIG. 12L). Altogether, BATF is a key target of Regnase-1 to limit CD8⁺effector T cell accumulation and mitochondrial metabolism in tumor immunity.

For the luciferase assay, the full-length 3′ UTR constructs of Batf (MmiT031430-MT06), Il2 (MmiT092987-MT06) and Il4 (MmiT092992-MT06) mRNAs were purchased from GeneCopoeia, each containing two luciferase genes: firefly luciferase gene for 3′ UTR of the targeted gene, and Renilla luciferase gene as an internal control. The cDNA of wild-type Regnase-1 (Dharmacon MMM1013-202800061) was cloned into the pMIG-II vector. The D141N mutant Regnase-1 was generated by site-directed mutagenesis using the KOD Hot Start DNA Polymerase (Millipore 71086). HEK293T cells were transfected with 3′ UTR construct of interest together with wild-type or D141N mutant Regnase-1 expression plasmid or empty control plasmid. At 48 h after transfection, cells were lysed and luciferase activities in the lysates were determined using the Luc-Pair Duo-Luciferase Assay Kit (GeneCopoeia LF002) according to manufacturer's instructions.

### Example 12

### Regnase-1-BATF Axis Shapes Mitochondrial Metabolism and Effector Responses

The results from Example 11 suggest that aberrant BATF expression is, at least in part, responsible for the excessive accumulation of Regnase-1-null OT-I cells. BATF co-deletion also elevated cell death (active caspase-3 staining) of Regnase-1-null OT-I cells, although not to the same extent as BATF deletion alone (FIG. 17E). In contrast, BATF/Regnase-1-null OT-I cells still had increased TCF-1 expression compared to wild-type OT-I cells (FIG. 17F), suggesting that the increased TCF-1 expression in Regnase-1-null OT-I cells is not dependent on aberrant BATF expression. Moreover, consistent with the context-dependent roles of BATF in mediating antiviral CDS⁺ T cell responses^{9,85}, BATF co-deletion blocked the increased IFN-y production in Regnase-1-null OT-I cells (FIG. 17G). In pmel-1-mediated ACT model, BATF co-deletion significantly decreased the therapeutic efficacy of Regnase-1-null cells against melanoma (FIG. 17H). Therefore, Regnase-1 targets BATF to impair the accumulation and effector function of CDS⁺ T cells in tumor immunity, but not TCF-1 expression.

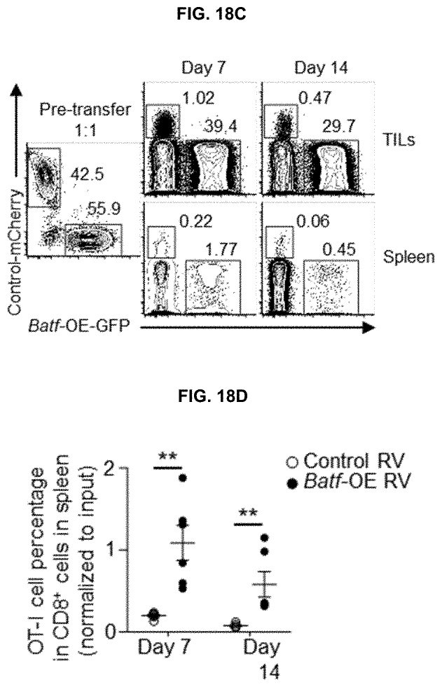

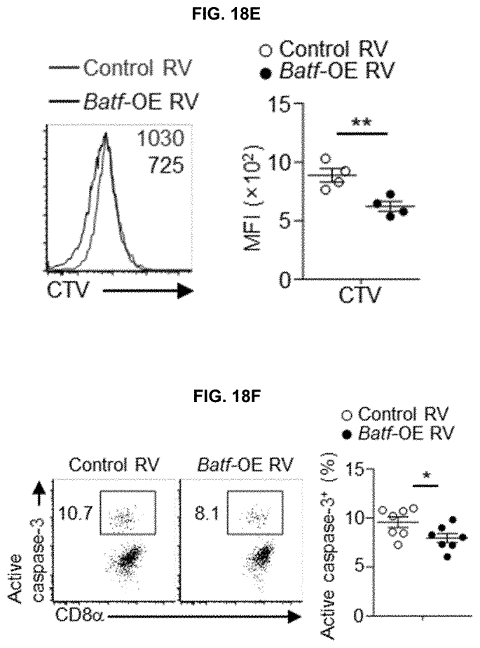

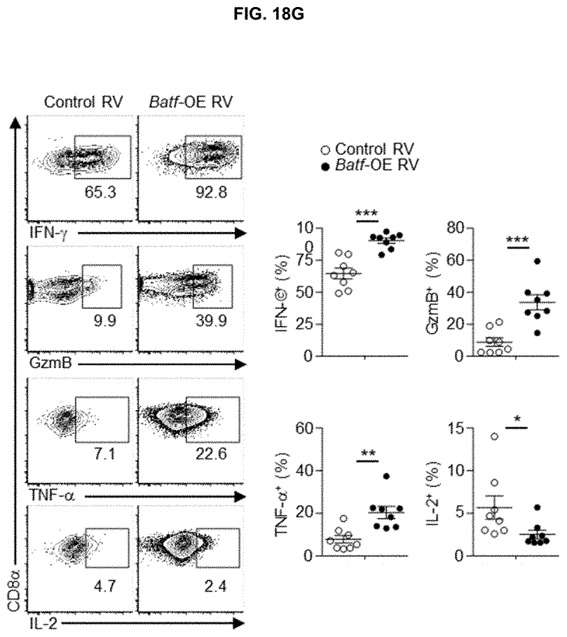

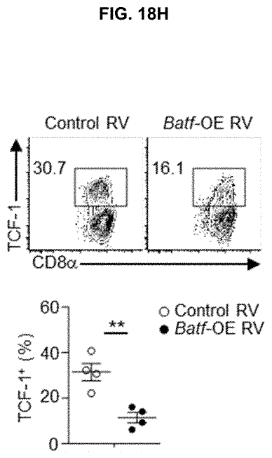

To examine upstream signals that drive aberrant BATF expression in Regnase-1-null OT-I cells, BATF expression was measured in response to TCR, IL-2 or IL-21 stimulation. Stimulation with aCD3 antibody and IL-2 (to a lesser extent) induced aberrant BATF expression in Regnase-1-null OT-I cells compared to wild-type cells (FIG. 18A). Although IL-21 can sustain BATF expression in antiviral T cells⁸⁶, IL-21 did not affect BATF expression in Regnase-1-null cells (FIG. 18A). Given the specific regulation of BATF expression by Regnase-1 and immune signals, it was hypothesized that BATF is an important rheostat in mediating antitumor CDS⁺ T cell effector responses by serving as a limiting factor in this process. To this end, wild-type OT-I cells were transduced with BATF and transferred them into tumor-bearing mice (FIG. 18B). BATF overexpression improved cell accumulation in the spleen (FIGS. 18C, 18D) and even more profoundly in the tumor (FIG. 4K, FIG. 18C). Accordingly, BATF-overexpressing OT-I cells in the tumor had increased cell proliferation and modestly reduced active caspase-3 expression (FIG. 18E, 18F), and produced more effector molecules, including IFN-y, GzmB and TNF-a but not IL-2 (FIG. 18G). In contrast but consistent with the role of BATF in promoting effector cell differentiation, TCF-1 expression was reduced in BATF-overexpressing OT-I cells (FIG. 18H). These results indicate that Regnase-1 suppresses TCR and IL-2-induced BATF expression and reveal BATF as an important rheostat in mediating antitumor effector responses in TME.

For viral transduction to overexpress BATF, the coding sequence of Batf (Addgene # 34575) was subcloned into pMIG-II retroviral vector (Addgene # 52107), which was co-transfected into Plat-E cells with the helper plasmid pCL-Eco (Addgene # 12371) for the production of retrovirus.

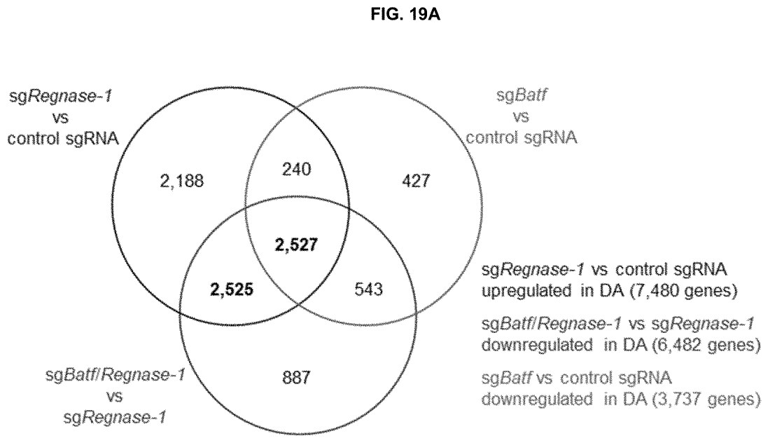

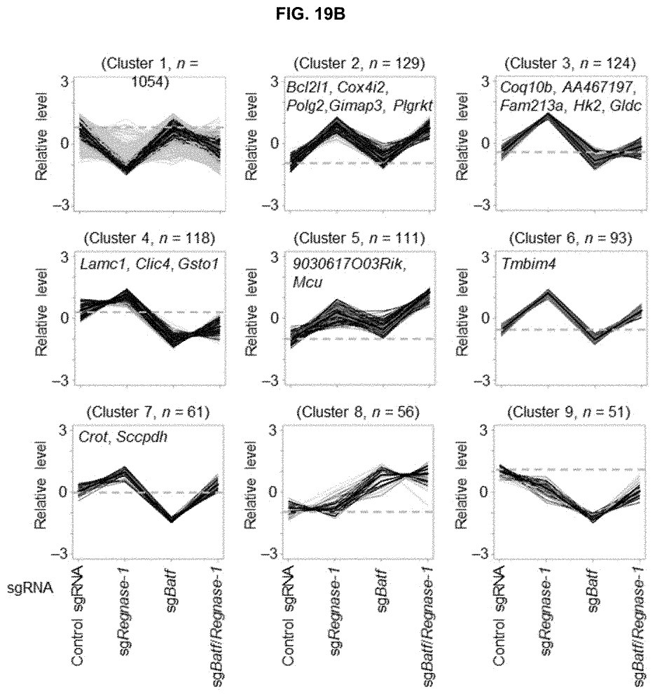

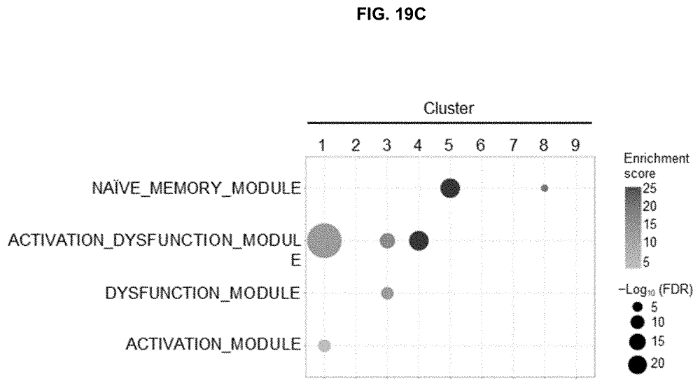

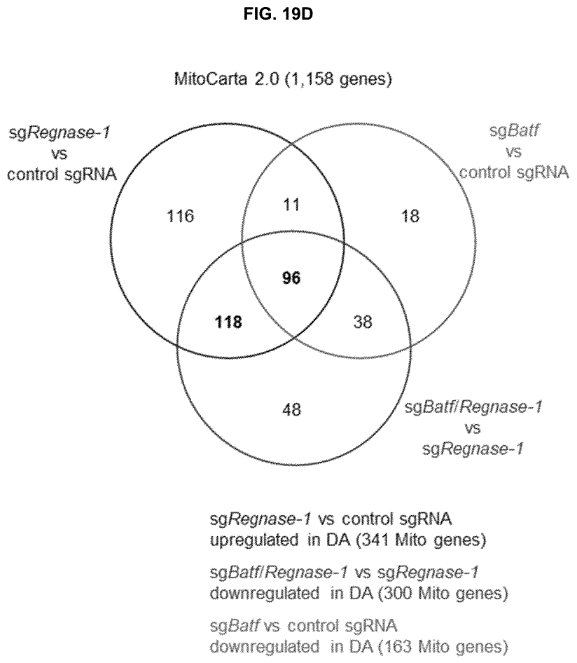

BATF is a pioneer factor that controls chromatin accessibility, which allows subsequent binding by other transcription factors^{9,75}. To directly determine the contribution of aberrant BATF expression to the altered chromatin accessibility in Regnase-1-null cells, ATAC-Seq analysis was performed by comparing wild-type, Regnase-1-null, BATF-null, and BATF/Regnase-1-null cells isolated from TILs. 7,480 genes were identified with significantly increased chromatin accessibility in Regnase-1-null cells as compared to wild-type cells (FIG. 19A), and remarkably, BATF co-deletion reversed the upregulated chromatin accessibility for a large proportion of these genes (5,052 in total) (FIG. 19A). In addition, 2,527 among these 5,052 genes showed significantly downregulated chromatin accessibility in BATF-null cells as compared to wild-type cells (FIG. 19A). These results indicate that aberrant BATF expression drives increased chromatin accessibility in Regnase-1-null tumor-specific T cells.

To further dissect BATF-dependent and independent pathways associated with Regnase-1 function, transcriptome analysis was performed by comparing wild-type, Regnase-1-null, BATF-null, and BATF/Regnase-1-null cells isolated from TILs. Principal component analysis (PCA) of global expression profiles revealed that compared with Regnase-1-null cells, BATF/Regnase-1-null OT-I cells showed considerably less variance from wild-type cells (FIG. 4L), suggesting the partial correction of Regnase-1-null-induced gene expression programs by BATF co-deletion. To identify unique gene modules regulated by BATF in Regnase-1-null cells, weighted gene correlation network analysis (WGCNA)¹² was applied and differentially expressed genes were grouped into nine distinct co-expression clusters (FIG. 19B). Remarkably, four clusters (clusters 3, 4, 6 and 7) showed upregulated gene expression in the absence of Regnase-1 that was blocked or partially blocked by BATF co-deletion in BATF/Regnase-1-null cells. Within these 4 clusters, gene expression in cells lacking BATF alone was either largely unaltered (clusters 3 and 6) or reduced (clusters 4 and 7), compared with wild-type controls (FIG. 19B). In addition, gene expression in cluster 1 was downregulated in Regnase-1-null cells but partially rectified by BATF co-deletion (FIG. 19B). In total, these 5 clusters identified Regnase-1 deletion-induced transcriptional programs with complete or partial dependence on BATF. Functional enrichment analysis using gene modules associated with different functional states of CD8⁺ T cells in tumor immunity', as described above, revealed that these clusters were enriched with genes in the activation and/or dysfunction module (FIG. 19C), thereby reinforcing the abovementioned role of BATF in mediating the effector function of Regnase-1-null OT-I cells. The remaining four clusters (2, 5, 8 and 9) contained gene profiles that were altered in Regnase-1-null cells but not rescued by BATF co-deletion (FIG. 19A). Interestingly, clusters 5 and 8 were enriched with genes in the naïve or memory module (FIG. 19C), which, together with the analysis of TCF-1 expression described above (FIGS. 17F, 18H), support the idea that the increased naive/memory-like gene signatures in Regnase-1-null cells are largely independent of BATF. Altogether, aberrant BATF expression drives effector-associated, but not naive/memory-like, transcriptional programs in Regnase-1-null OT-I cells, further supporting the dual roles of Regnase-1 in coordinating T cell effector function and persistence in antitumor immunity.

Next the functional pathways underlying Regnase-1-mediated protective immunity were determined in tumor-specific CDS⁺ T cells. Of note, functional enrichment of the top-ranking depleted genes of the secondary in vivo genome-scale CRISPR-Cas9 mutagenesis screening revealed that oxidative phosphorylation (OXPHOS) hallmark was the top-ranked pathway (FIG. 11B), suggesting a possible role for mitochondrial oxidative metabolism in mediating the excessive accumulation of Regnase-1-null OT-I cells. In support of this notion, increased mitochondrial OXPHOS metabolism has been shown to correlate with improved fitness of effector T cells and their antitumor activity^{12,14,83,84,} although the negative signals controlling mitochondrial metabolism, especially in the TME, remain elusive. It was also noted that OXPHOS was the top-ranking gene set enriched in tumor-infiltrating Regnase-1-null OT-I cells relative to wild-type cells (FIG. 9A and FIG. 11C). Therefore mitochondrial profiles and oxidative metabolism were measured in Regnase-1-null OT-I cells, which had increased mitochondrial fitness, as indicated by increased mitochondrial mass, membrane potential (FIG. 4B) and volume (FIG. 11D). They also had significantly higher basal and maximal oxygen consumption rate (OCR, indicative of OXPHOS activity) (FIG. 4C), indicating enhanced oxidative metabolism. Importantly, compared with Regnase-1-null cells, BATF/Regnase-1-null cells had downregulation of OXPHOS and cell cycling-associated hallmarks (FIGS. 12J, 12K), consistent with the role of BATF in mediating Regnase-1-null cell accumulation. In further support of a role of BATF in promoting oxidative metabolism of Regnase-1-null OT-I cells, BATF co-deletion largely blocked the increased mitochondrial mass and membrane potential in Regnase-1-null OT-I cells, but not in wild-type cells, at days 5 and 7 after adoptive transfer (FIG. 4M). Conversely, BATF overexpression was sufficient to upregulate mitochondrial mass and membrane potential (FIG. 4N). These results collectively reveal a role of BATF in linking Regnase-1 function and mitochondrial fitness.

To understand the molecular basis for Regnase-1 and BATF-mediated regulation of mitochondrial fitness, first the ATAC-Seq data of wild-type, Regnase-1-null, BATF-null, and BATF/Regnase-1-null OT-I cells from TILs were mined for altered chromatin accessibility of mitochondrial genes defined in the MitoCarta 2.0 database^{11,12}. A total of 341 mitochondrial genes showed significantly upregulated chromatin accessibility in the absence of Regnase-1 (FIG. 19D), 214 of which were blocked by BATF co-deletion in BATF/Regnase-1-null cells (FIG. 19D). Moreover, 96 among these 214 genes showed significantly downregulated chromatin accessibility in BATF-null cells as compared to wild-type cells (FIG. 19D). These results further support a crucial contribution of BATF to the enhanced mitochondrial function in the absence of Regnase-1. Second, the expression of mitochondrial genes was examined in the transcriptome analysis of wild-type, Regnase-1-null, BATF-null, and BATF/Regnase-1-null cells isolated from TILs. A total of 18 mitochondrial genes were identified in clusters 2-7 of WGCNA that contained upregulated genes in the absence of Regnase-1, including 11 genes in clusters 3, 4, 6 and 7 with complete or partial dependence on BATF (FIG. 19B). Thus, the aberrant mitochondrial gene expression in Regnase-1-null cells is at least partially dependent on BATF. Altogether, these results reveal a Regnase-1-BATF axis in reprogramming mitochondrial metabolism of CD8⁺ T cells, and highlight important contributions of increased BATF activity to altered chromatin accessibility and transcript expression of mitochondrial genes in Regnase-1-null cells.

### Example 13

### Identification of Additional Targets for Adoptive Cell Transfer (ACT)

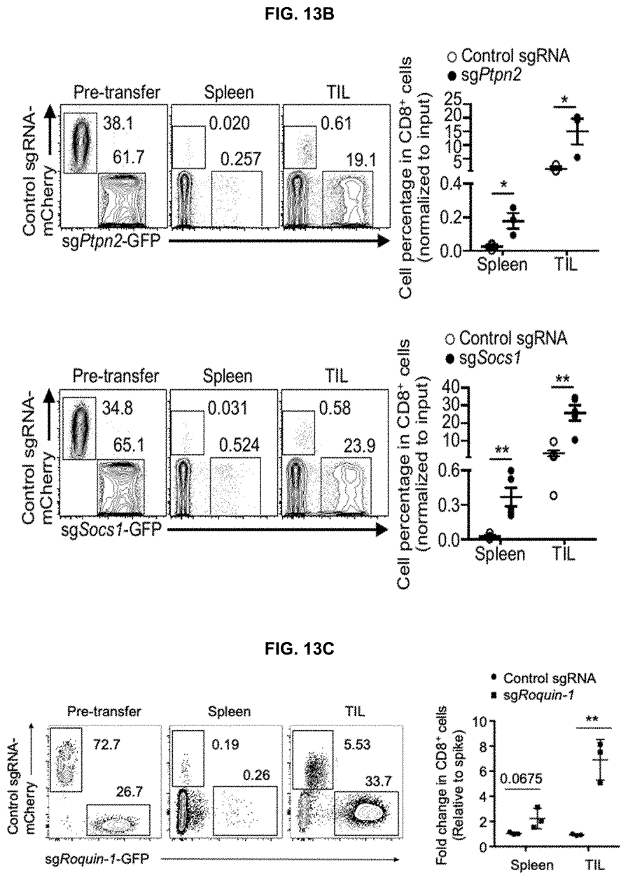

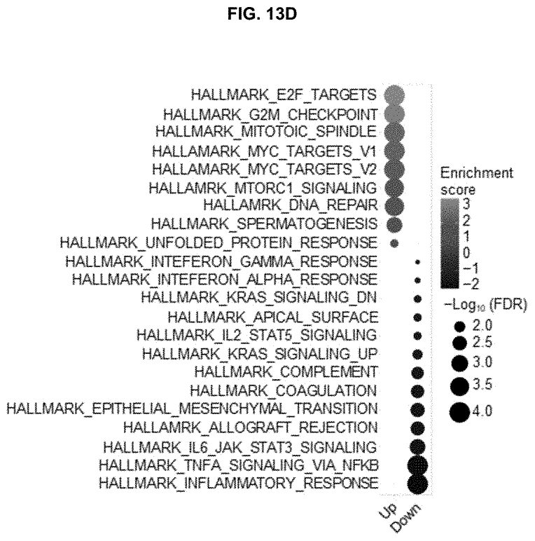

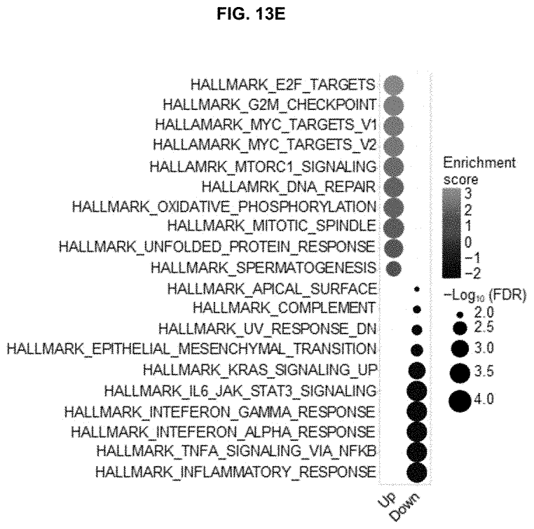

To further explore the therapeutic potential of Regnase-1-null CD8⁺ T cells, the top enriched genes were examined in TILs in the genome-scale CRISPR-Cas9 mutagenesis screening. It was reasoned that if a candidate gene could further improve the accumulation of Regnase-1-null OT-I cells, its sgRNAs should be enriched in TILs. The top candidate genes were chosen for further analysis, including Ptpn2, Socs1 and Agps (FIG. 11A). The effects of deleting these genes were validated on further enhancing the accumulation of Regnase-1-null OT-I cells in both the periphery and tumor at 7 days after adoptive transfer (FIGS. 13A, 4O, 20A). Among genes tested, deletion of PTPN2 or SOCS1 showed potent effects on improving the accumulation of Regnase-1-null OT-I cells in the periphery and tumor (FIGS. 4H and 4I). Deletion of PTPN2, SOCS1 or Roquin-1 (encoded by Rc3h1) itself also resulted in increased accumulation of OT-I cells in both the tumor and periphery (FIGS. 13B and 13C). Also, deletion of PTPN2 or SOCS1 alone resulted in a modestly increased accumulation of OT-I cells in the tumor (FIG. 40). Next, transcriptome analysis was performed on PTPN2/Regnase-1-null, SOCS 1/Regnase-1-null, and control Regnase-1-null OT-I cells, and GSEA revealed the enrichment of cell cycle and metabolic hallmark pathways in PTPN2/Regnase-1-null and SOCS1/Regnase-1-null cells relative to the Regnase-1-null controls (FIGS. 13D and 13E), consistent with the increased accumulation in tumor immunity.

It was found that expression of PTPN2 and SOCS1 was, unlike BATF, not affected by Regnase-1 deletion (FIG. 20B). While Regnase-1-null OT-I cells had markedly elevated mitochondrial fitness, deletion of PTPN2 or SOCS1 alone did not affect or slightly increased mitochondrial mass and membrane potential, respectively (FIGS. 4P, 4Q). Also, in PTPN2- or SOCS1-null OT-I cells, the additional deletion of Regnase-1 still elevated these mitochondrial profiles (FIGS. 4P, 4Q). Furthermore, deletion of PTPN2 or SOCS1 did not affect BATF expression in either wild-type or Regnase-1-null OT-I cells (FIG. 4R). Interestingly, in contrast to the elevated TCF-1 expression in Regnase-1-null OT-I cells, loss of PTPN2 or SOCS1 alone significantly reduced TCF-1 expression as compared to wild-type cells (FIG. 4S), whereas Regnase-1 co-deletion still increased TCF-1 expression in PTPN2- or SOCS1-null cells (FIG. 4S). These comparative analyses reveal largely discrete mechanisms exerted by PTPN2 or SOCS1 in comparison to Regnase-1, including mitochondrial fitness and regulation of BATF and TCF-1 expression. In further support of this notion, PCA plot of the transcriptome profiles revealed largely distinct patterns for Regnase-1-null, PTPN2-null and SOCS1-null OT-I cells, which were further segregated from the combined loss of PTPN2 and Regnase-1 or of SOCS1 and Regnase-1 (FIG. 4T), thereby highlighting differential gene expression programs.

Given the increased accumulation of tumor-infiltrating PTPN2/Regnase-1-null and SOCS1/Regnase-1-null CD8⁺ T cells as compared to Regnase-1-null cells (FIG. 40), their therapeutic efficacy was assessed against B16-F10 melanoma using the pmel-1 TCR-transgenic system. As compared to Regnase-1 deletion alone, PTPN2/Regnase-1-null and SOCS1/Regnase-1-null pmel-1 T cells exhibited additional effects to delay tumor growth (FIG. 4U).

Altogether, the CRISPR-Cas9 mutagenesis screening identifies additional potential targets to combine with Regnase-1 deletion for combinatorial cancer immunotherapy.

As shown in the Examples described above, by integrating focused and genome-scale CRISPR screening, bioinformatic analyses and extensive experimental validation, it is revealed that tumor-specific CD8⁺ T cells can be reprogrammed in the TME to acquire extensive accumulation and increased naive/memory cell-associated features (more quiescence, better survival and naive/memory cell-associated gene signatures) for long-term persistence, while retaining robust effector function (FIG. 14). Regnase-1 is identified unbiasedly by the in vivo pooled CRISPR-Cas9 mutagenesis screening as a major regulator to be targeted to unleash this unique reprogramming in the TME, resulting in marked therapeutic efficacy against both solid and blood cancers in ACT. The specific transcriptional adaptation of Regnase-1-null CD8⁺ T cells in the TME highlights a previously unappreciated function of Regnase-1 after initial T cell activation^{15,16}, to enable precise temporal and spatial control of T cell responses.

The results highlight that Regnase-1 restrains mitochondrial metabolism and effector responses through a key gene target BATF, which is identified through a secondary in vivo genome-scale CRISPR-Cas9 screening in immune cells. The Examples presented above reveal Regnase-1 as a major negative regulator of BATF and a previously unappreciated role of BATF as a limiting factor for programming effective antitumor responses, in part through shaping mitochondrial metabolism, thereby contributing to the understanding of the context-dependent roles of the pioneer factor BATF in adaptive immunity^{9,76,85}. The genome-scale CRISPR-Cas9 screening also reveals PTPN2, SOCS1 and Roquin-1 (encoded by Rc3h1) as potential targets to be deleted alone or to combine with Regnase-1 deletion to boost antitumor immunity.

### Example 14

### Regnase-1 Deficient Human CAR-T Cells have Improved Survival and Function

Generation of Regenase-1 knockout human CAR-T cells

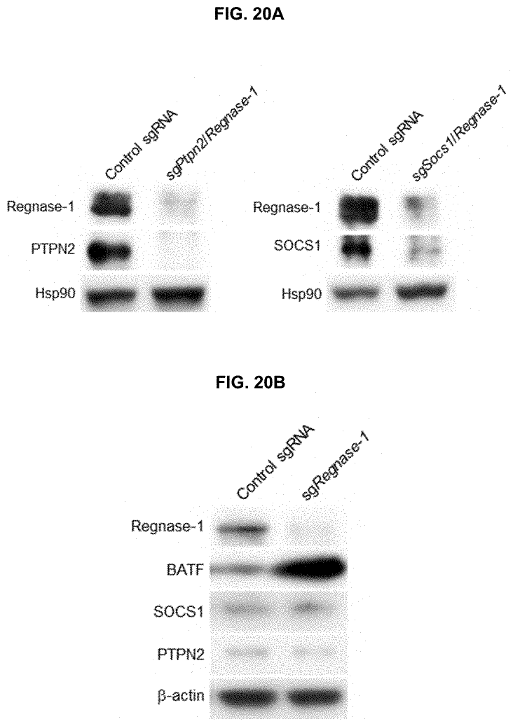

First, the gRNAs to knock out Regenase-1 in human CD4 and CD8 CAR-T cells were designed in silico based on selected off-target profiles shown in Table 2. FIG. 21 shows a schematic of the gRNA binding sites along Regnase-1.

In Table 2, “long_0” refers to the number of sites in genome that are an exact match to the full-length (“long”) 23nt gRNA target sequence listed in the table, including the target site; “long_1” refers to the number of sites in genome that contain up to 1 mismatch in the 23nt gRNA target sequence listed in the table, including the target site; “long_2” refers to the number of sites in genome that contain up to 2 mismatches in the 23nt gRNA target sequence listed in the table, including the target site; and “short_0” refers to the number of sites in genome that match to the 15nt fragment (“short”) at the 3′ end of the gRNA target sequence listed in the table, including the target site.

TABLE 2
Details of gRNAs designed for Regnase-1 knockout

| Name | gRNA | long_0 | long_1 | long_2 | short_0 | SNP |
|---|---|---|---|---|---|---|
| gRNA-1 | TTCACACCATCACGACGCGTNGG | 1 | 1 | 1 | 1 | NA |
| | (SEQ ID NO: 29) | | | | | |
| gRNA-2 | TGGGGGCAGCTTGGCCGCTCNGG | 1 | 1 | 1 | 3 | NA |
| | (SEQ ID NO: 30) | | | | | |
| gRNA-3 | TATGCCCCCTGATGACCCACNGG | 1 | 1 | 1 | 21 | NA |
| | (SEQ ID NO: 31) | | | | | |
| gRNA-4 | AAGGAGGTCTTCTCCTGCCGNGG | 1 | 1 | 1 | 8 | NA |
| | (SEQ ID NO: 32) | | | | | |
| gRNA-5 | GTGATGGGCACGTCGGGCCGNGG | 1 | 1 | 1 | 1 | NA |
| | (SEQ ID NO: 33) | | | | | |
| gRNA-6 | CAGCTCCCTCTAGTCCCGCGNGG | | | | | |
| | (SEQ ID NO: 34) | | | | | |
| control | GCUUGUGGAUGUUGCGGAAGNGG | | | | | |
| gRNA | (SEQ ID NO: 35) | | | | | |

The sequences for gRNAs 1-5 were designed by CAGE while the sequence for gRNA-6 was selected from portals.broadinstitute. org/gpp/public/analysi s-tools/sgrna-design.

Knockout of Regenase-1 gene in human CAR-T cells by CRISPR-Cas9

The following protocol was used to knock out the Regenase-1 gene in human CD8 T cells.

On Day 0, human na-ve CD4 or CD8 T cells were isolated by sorting CCR7⁺ CD45RA⁺ CD45RO⁻CD4⁺ T cells or CCR7⁺ CD45RA⁺ CD45RO⁻CD8⁺ T cells. The isolated CD4 and CD8 T cells were activated by plating on CD3/CD28 coated plates.

On Day 1, CD19-CAR transduction using a lentiviral vector was used to introduce CD19-CAR into the activated CD4 or CD8 T cells.

On Day 2, one of the six candidate gene gRNAs or control non-targeting gRNA in complex with Cas9 protein were electroporated into the T cells collected from the culture plates according to the following protocol.

Purified Cas9 protein and guide RNA oligonucleotides were combined to form a ribonucleoprotein (RNP) complex. Specifically, the selected single guide RNA (sgRNA) was re-suspended at 100μM (1.5 nmol in 15μl water) in approximately 13-14 μL aliquots, and the Cas9 protein was prepared at a concentration of 40 μM (40 pmol/μL) in approximately 10 μL aliquots.

The RNP was prepared by mixing the gRNA and Cas9 protein following these conditions in PCR tubes: 1.8 μL (100 pmol/μ1, 180 pmol) gRNA is mixed with 1 μL (40 pmol/μL, 40 pmol) Cas9 protein, for a total volume of 2.8 pl. After briefly mixing, the RNP was incubated at room temperature for 10 minutes before being placed at 4° C. until ready for use.

The T cells were resuspended in complete RPMI medium and counted. The T cells were centrifuged for 5 min at 1300rpm, aspirated, washed with PBS once, and resuspended at 25M/m1 in Lonza electroporation buffer P3 from the Lonza Amaxa™ P3 primary cell 96-well Nucleofector™Kit (Cat. No. V4SP-3096). The T cells were resuspended at a ratio of 20 μl Lonza electroporation buffer P3 per 0.5 million cells. The T cells were briefly mixed by pipetting with 2.8 μl RNP mixture. The RNP and cells mixture was transferred to an electroporation cuvette. Immediately after electroporation, 80μl of pre-warmed media (without cytokine) was added to each cuvette. The cells were allowed to rest for 15 mins at 37° C. in the incubator while remaining in the cuvettes. After 15 mins, cells were moved to 24-well tissue culture plate by adding the cell suspension directly to one well containing 500 μL complete RPMI medium with IL-7 and IL-15.

For optimal editing, 0.5 million T cells were electroporated per well using a Lonza 4D 96-well electroporation system (Lonza 4D Nucleofector™ Core Unit) with pulse code EO115. Electroporation was completed until green crossing was observed on the samples. Alternate cell concentrations from 200,000 up to 2 million cells per well resulted in lower transformation efficiencies.

On Day 6, the T cell status was checked and the ability of the T cells to be co-cultured with Raji cells, including killing of the Raji cells, was measured for 24 hrs and 48 hrs.

On Day 10, samples of the T cells were taken for Western blotting and deep sequencing, or used for in vivo study.

Characterization of the CD4 and CD8 Regnase-1-null CAR-T cells

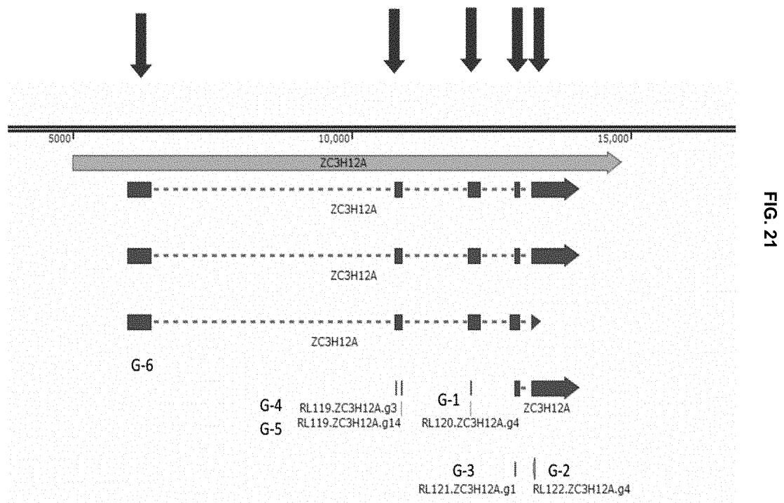

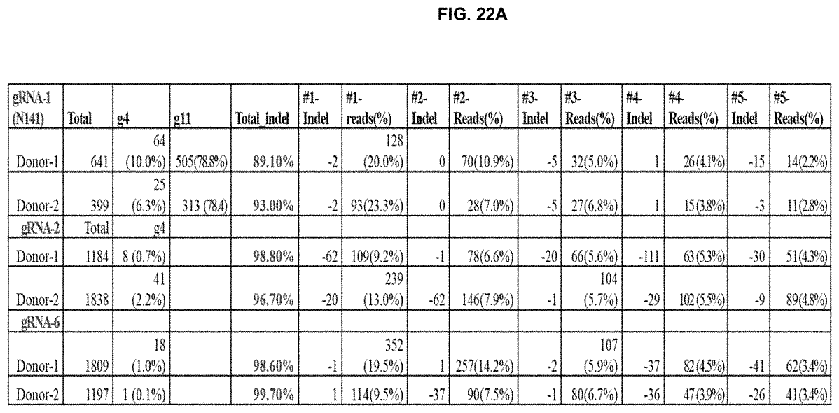

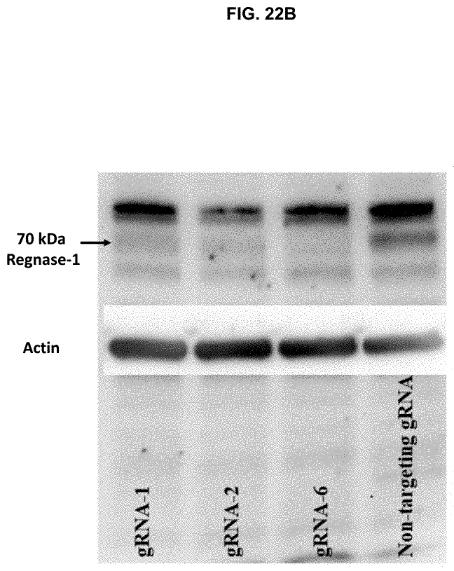

The deep sequencing results are shown in FIG. 22A. The total indel achieved with gRNA-1, gRNA-2 and gRNA-6 ranged from 89.1% to 99.7%.

As shown in FIG. 22B, the gRNA-6 oligonucleotide led to a knock-out of Regnase-1 as measured by protein level, while gRNA-1 and gRNA-2 led to a partial knock out of Regenase-1 protein level. As seen in FIG. 21, gRNA-1 targets the N141 site of Regnase-1, which is very important to its RNase function. Based on these results, gRNA-1 and gRNA-6 were selected for further analysis.

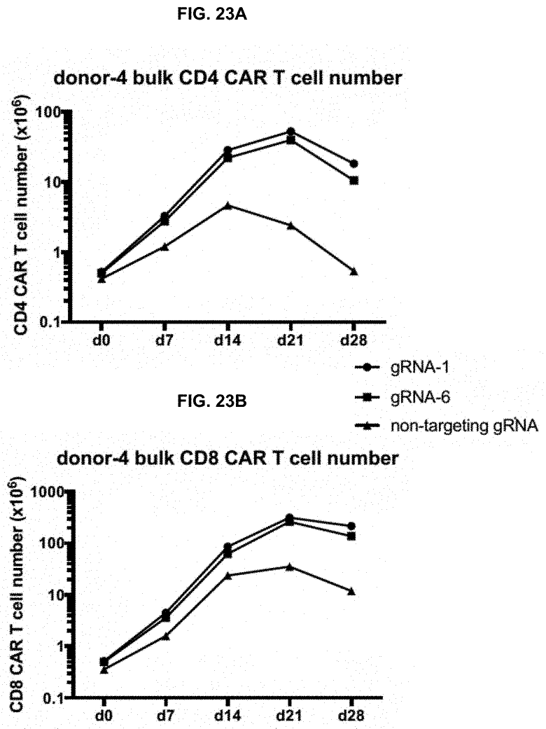

The human Regnase-1-null CAR-T cells were tested to see if they could be multi-activated with bulk T cells. The CAR-T cells were stimulated with irradiated Raji cells at a ratio of 2:1 every 7 days. T cell phenotypes were measured 24 hours after each stimulation. FIGS. 23A-23B show that human Regnase-1-null CAR-T cells had improved survival ex vivo. FIG. 23A shows improved survival of human CD4 Regnase-1-null CAR-T cells with the two selected guide RNAs gRNA1 and gRNA6. FIG. 23B shows improved survival of human CD8 Regnase-1-null CAR-T cells with the two selected guide RNAs gRNA1 and gRNA6.

The proliferative and apoptotic properties of the human CD4 and CD8 Regnase-1-null CAR-T cells were determined ex vivo. The proliferative capabilities were measured by cell-trace violet (CTV). Before a third round of activation, the CD4 and CD8 Regnase-1-null CAR-T cells were labeled with CTV. Seventy-two hours after activation, the T cell proliferation was measured. Cell apoptosis was measured 72 hours after a third round of stimulation with irradiated Raji tumor cells. FIGS. 24A-24B show that human Regnase-1-null CAR-T cells have improved proliferation (FIG. 24A) and reduced apoptosis (FIG. 24B) ex vivo.

The ability of the Regnase-1-null CAR-T cells to generate different types of memory T cells was studied. The differentiation status of CD4 and CD8 Regenase-1-null CAR-T cells was determined after each of three rounds of stimulation with irradiated Raji tumor cells. The differentiated T cells were sorted based on CCR7 and CD45R0 expression into the following four groups: (1) naive (CD45RO⁻CCR7⁺), (2) central memory (CD45RO⁺CCR7⁺), (3) effector memory (CD45RO⁺CCR7⁻), and (4) effector (CD45RO⁻CCR7⁻). FIGS. 25A-25B show that human CD4 Regnase-1-null (FIG. 25A) and CD8 Regnase-1-null (FIG. 25B) CAR-T cells have more memory subsets upon antigen activation ex vivo in comparison to control wildtype CD4 and CD8 CAR-T cells.

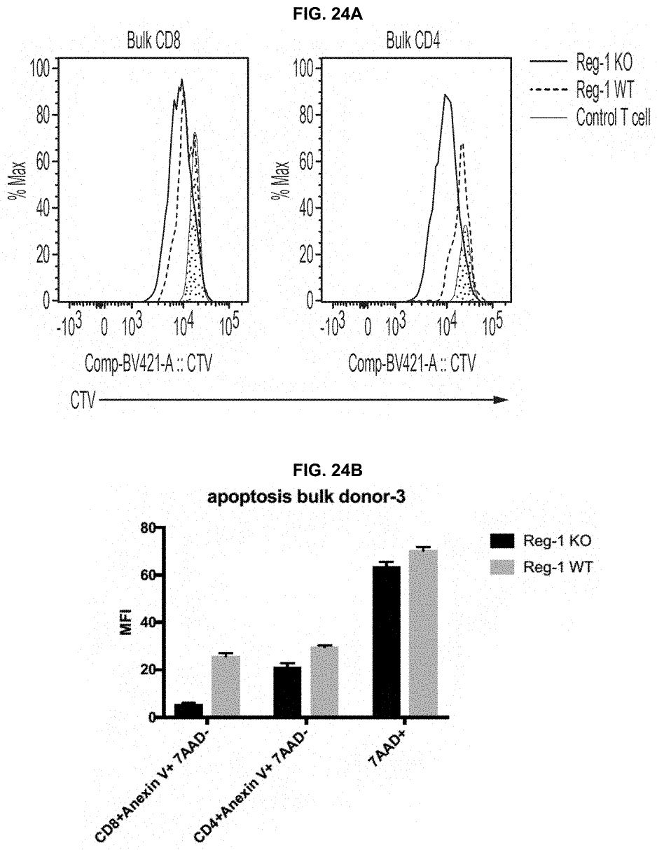

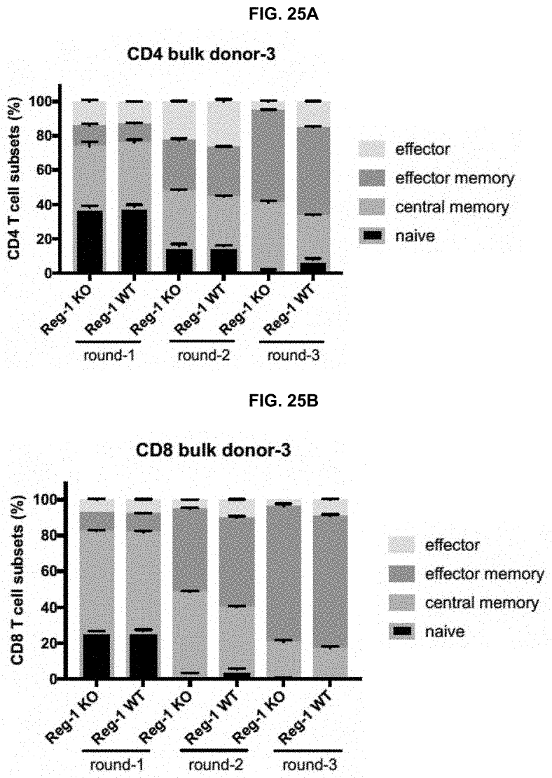

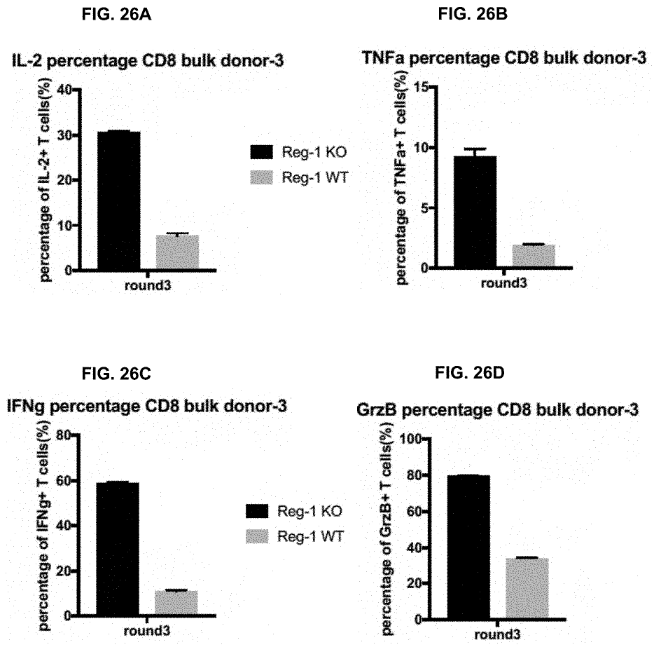

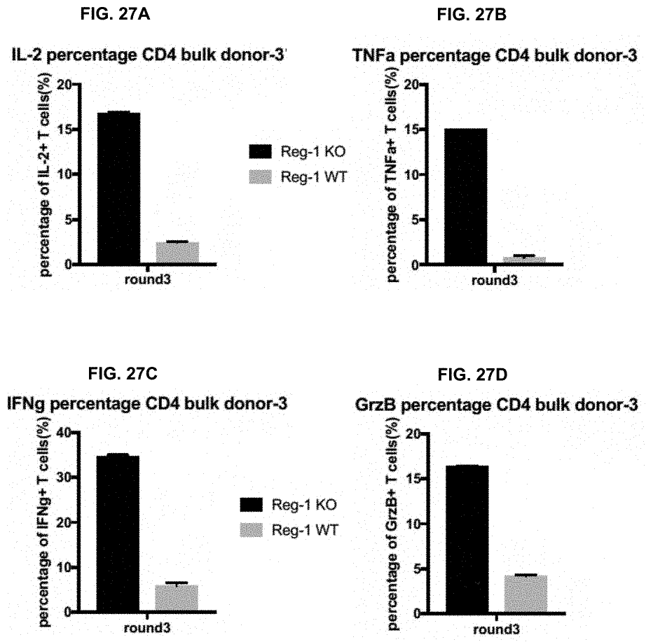

The effect of the Regnase-1 knockout on cytokine production was then measured. The levels of selected cytokines were measured 24 hours after the third stimulation with irradiated Raji tumor cells. FIGS. 26A-26D show that human CD8 Regnase-1-null CAR-T cells secrete more cytokines ex vivo, specifically IL-2 (FIG. 26A), TNFa (FIG. 26B), IFN-gamma (FIG. 26C), and GrzB (FIG. 26D). 27A-27D show that human CD4 Regnase-1-null CAR-T cells secrete more cytokines ex vivo, specifically IL-2 (FIG. 27A), TNFa (FIG. 27B), IFN-gamma (FIG. 27C), and GrzB (FIG. 27D).

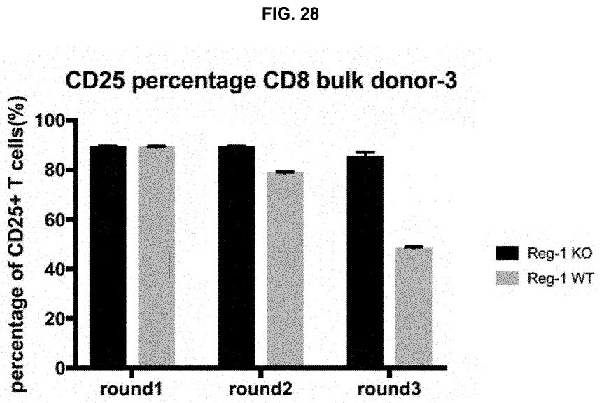

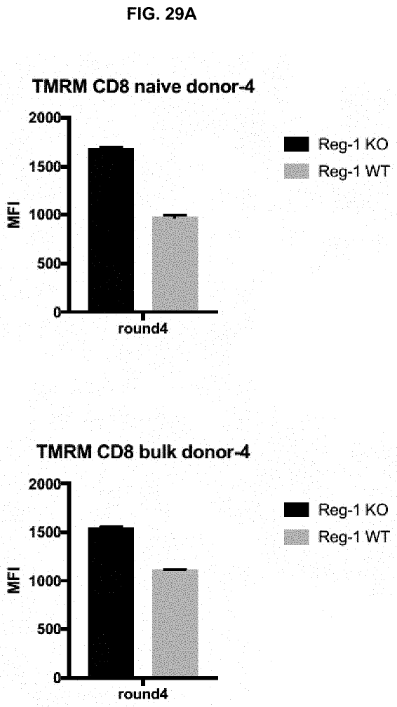

The effect of the Regnase-1 knockout on the CD25 activation status of the Regnase-1-null CD8 CAR-T cells was then measured. The CD25 activation status of the cells was measured 24 hours after each of three rounds of stimulation with irradiated Raji tumor cells. FIG. 28 shows that CD8 Regnase-1-null CAR-T cells are hyper-active after the third round of stimulation ex vivo.

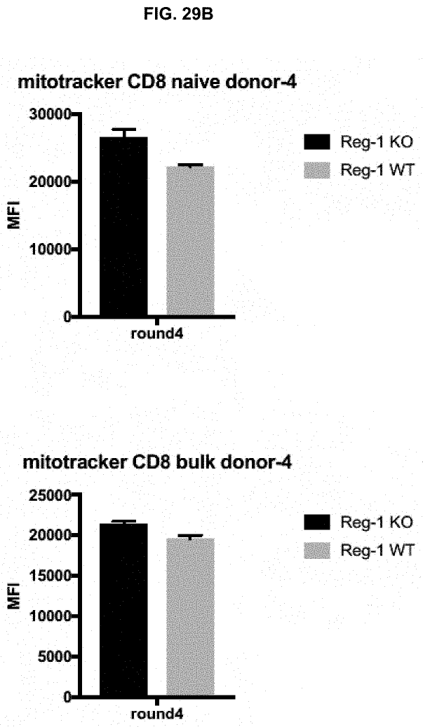

The effect of the Regnase-1 knockout on the mitochondrial activity of the Regnase-1-null CD8 CAR-T cells was then measured. The mitochondrial activity of the cells was measured 24 hours after the third stimulation with irradiated Raji tumor cells. FIGS. 29A-29B show that CD8 Regnase-1-null CAR-T naive (top panel) and bulk (bottom panel) cells have upregulated mitochondrial activity ex vivo as measured by TMRM (FIG. 29A) and mitotracker (FIG. 29B).

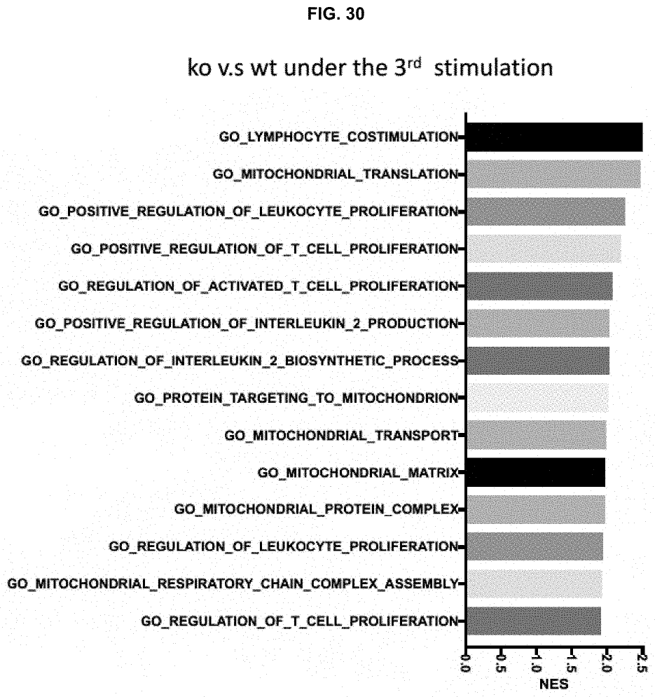

The effect of the Regnase-1 knockout on the activity of certain cell proliferation and mitochondrial-activity related genes was measured in Regnase-1-null CD8 CAR-T cells. FIG. 30 shows upregulation of certain genes related to T cell proliferation and mitochondrial activity in Regnase-1-null CAR-T cells ex vivo upon antigen stimulation by GSEA analysis.

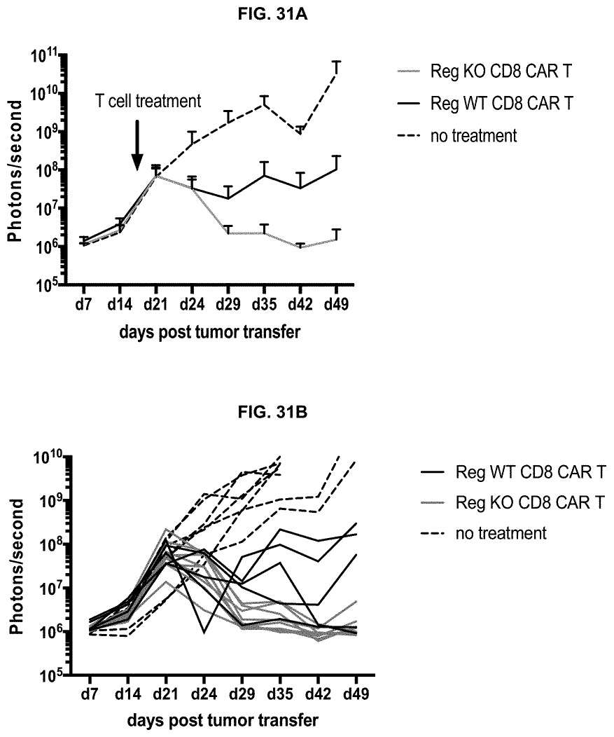

The effect of the Regnase-1 knockout on the ability of mice to survive tumor transfer with Raji cells was measured in vivo. The Raji cells were engrafted for two weeks in NSG mice and the tumors were observed by Xenogen imaging. Bulk Regnase-1-null CD8 CAR-T cells and wildtype CD8 CAR-T cells were then transferred into the mice at 1 million cells per mouse (approximately 50% CAR+). At the two-week mark, nine mice received the Regnase-1-null CD8 CAR-T cells, five mice received the wildtype CD8 CAR-T cells, and seven mice did not receive any treatment. The tumors were observed and quantified by Xenogen imaging every seven days after the T-cell treatment. FIGS. 31A-31B show that mice treated with Regnase-1-null CD8 CAR-T cells in vivo had lower tumor burden as indicated by the luciferase activity of each treatment group (FIG. 31A) and individual recipient (FIG. 31B).

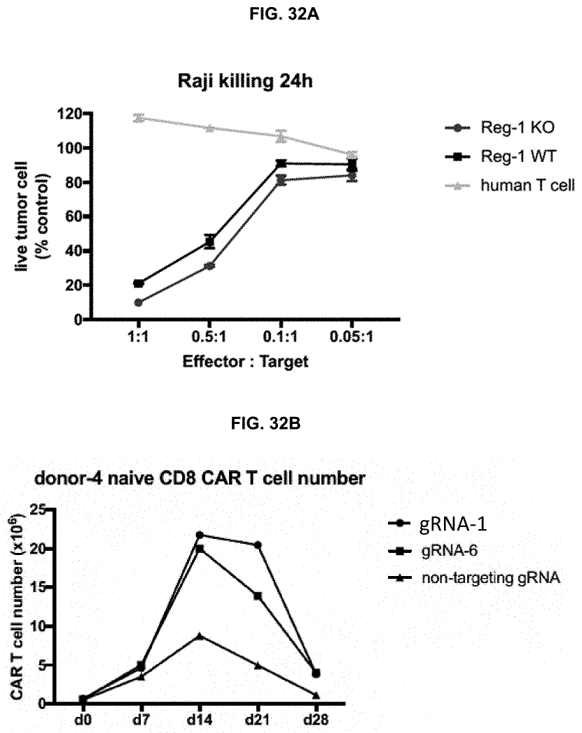

The effect of the Regnase-1 knockout on the cytotoxicity and survival of CAR-T cells was measured. For the ex vivo cytotoxicity assay, Regnase-1-null cells and wildtype CAR-T cells were incubated with Raji cells at different effector and target ratios. Twenty-four hours later, the number of live tumor cells was measured. For the ex vivo survival assay, na-ve CD8 T cells were used. The CAR-T cells were stimulated with irradiated Raji cells at a ratio of 2:1 every seven days. The T cell phenotypes were measured 24 hours after each stimulation. FIGS. 32A-32B show that human Regnase-1-null CAR-T cells had improved cytotoxicity (FIG. 32A) and improved survival (FIG. 32B) ex vivo.

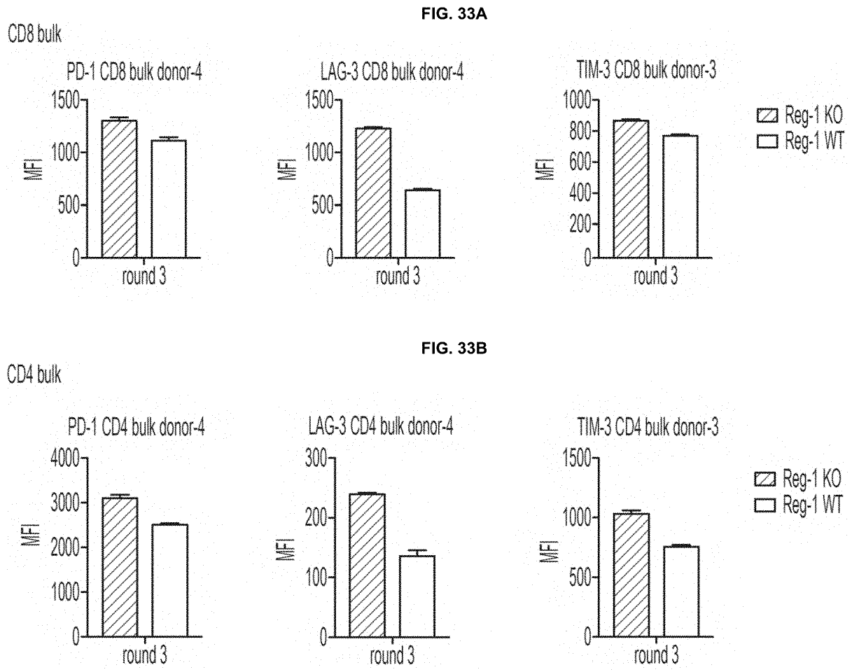

The effect of the Regnase-1 knockout on the expression of certain cell hyper-activity markers was measured in Regnase-1-null CD4 and CD8 CAR-T cells. The expression of exhaustion markers PD-1, LAG3, and TIM-3 was measured 24 hours after a third stimulation with irradiated Raji tumor cells. FIGS. 33A-33B show hyperactivation of CD8 Regnase-1-null (FIG. 33A) and CD4 Regnase-1-null (FIG. 33B) CAR-T cells ex vivo due to higher expression of the three exhaustion markers PD-1, LAG3, and TIM-3 in the Regnase-1-null CD8 and CD4 CAR-T cells.

### REFERENCES

1 Lim, W. A. & June, C. H. The Principles of Engineering Immune Cells to Treat Cancer. Cell 168, 724-740, doi:10.1016/j.ce11.2017.01.016 (2017).
2 Sadelain, M., Riviere, I. & Riddell, S. Therapeutic T cell engineering. Nature 545, 423-431, doi:10.1038/nature22395 (2017).
3 Gattinoni, L. et al. Acquisition of full effector function in vitro paradoxically impairs the in vivo antitumor efficacy of adoptively transferred CD8+T cells. J Clin Invest 115, 1616-1626, doi:10.1172/JCI24480 (2005).
4 Fraietta, J. A. et al. Disruption of TET2 promotes the therapeutic efficacy of CD19-targeted T cells. Nature 558, 307-312, doi:10.1038/s41586-018-0178-z (2018).
5 Schreiber, R. D., Old, L. J. & Smyth, M. J. Cancer immunoediting: integrating immunity's roles in cancer suppression and promotion. Science 331, 1565-1570, doi:10.1126/science.1203486 (2011).
6 Wei, J. et al. Targeting REGNASE-1 programs long-lived effector T cells for cancer therapy. Nature 576, 471-476, doi:10.1038/s41586-019-1821-z (2019).
7 Im, S. J. et al. Defining CD8+T cells that provide the proliferative burst after PD-1 therapy. Nature 537, 417-421, doi:10.1038/nature19330 (2016).
8 Leong, Y. A. et al. CXCRS(+) follicular cytotoxic T cells control viral infection in B cell follicles. Nat Immunol 17, 1187-1196, doi:10.1038/ni.3543 (2016).
9 Kurachi, M. et al. The transcription factor BATF operates as an essential differentiation checkpoint in early effector CD8+T cells. Nat Immunol 15, 373-383, doi:10.1038/ni.2834 (2014).
10 Singer, M. et al. A Distinct Gene Module for Dysfunction Uncoupled from Activation in Tumor-Infiltrating T Cells. Cell 166, 1500-1511 e1509, doi:10.1016/j.ce11.2016.08.052 (2016).
11 Calvo, S. E., Clauser, K. R. & Mootha, V. K. MitoCarta2.0: an updated inventory of mammalian mitochondrial proteins. Nucleic Acids Res 44, D1251-1257, doi:10.1093/nar/gkv1003 (2016).
12 Tan, H. et al. Integrative Proteomics and Phosphoproteomics Profiling Reveals Dynamic Signaling Networks and Bioenergetics Pathways Underlying T Cell Activation. Immunity 46, 488-503, doi:10.1016/j.immuni.2017.02.010 (2017).
13 Kishton, R. J., Sukumar, M. & Restifo, N. P. Metabolic Regulation of T Cell Longevity and Function in Tumor Immunotherapy. Cell Metab 26, 94-109, doi:10.1016/j.cmet.2017.06.016 (2017).
14 Buck, M. D., Sowell, R. T., Kaech, S. M. & Pearce, E. L. Metabolic Instruction of Immunity. Cell 169, 570-586, doi:10.1016/j.ce11.2017.04.004 (2017).
15 Matsushita, K. et al. Zc3h12a is an RNase essential for controlling immune responses by regulating mRNA decay. Nature 458, 1185-1190, doi:10.1038/nature07924 (2009).
16 Uehata, T. et al. Maltl-induced cleavage of regnase-1 in CD4(+) helper T cells regulates immune activation. Cell 153, 1036-1049, doi:10.1016/j.ce11.2013.04.034 (2013).
17 Zhou, P. et al. In vivo discovery of immunotherapy targets in the tumour microenvironment. Nature 506, 52-57, doi:10.1038/nature12988 (2014).
18 Platt, R. J. et al. CRISPR-Cas9 knockin mice for genome editing and cancer modeling. Cell 159, 440-455, doi:10.1016/j.ce11.2014.09.014 (2014).
19 Hogquist, K. A. et al. T cell receptor antagonist peptides induce positive selection. Cell 76, 17-27 (1994).
20 Bellone, M. et al. Relevance of the tumor antigen in the validation of three vaccination strategies for melanoma. J Immunol 165, 2651-2656 (2000).
21 Birsoy, K. et al. An Essential Role of the Mitochondrial Electron Transport Chain in Cell Proliferation Is to Enable Aspartate Synthesis. Cell 162, 540-551, doi:10.1016/j.ce11.2015.07.016 (2015).
22 Muri, J. et al. The thioredoxin-1 system is essential for fueling DNA synthesis during T-cell metabolic reprogramming and proliferation. Nat Commun 9, 1851, doi:10.1038/s41467-018-04274-w (2018).
23 Peng, M. et al. Aerobic glycolysis promotes T helper 1 cell differentiation through an epigenetic mechanism. Science 354, 481-484, doi:10.1126/science.aaf6284 (2016).
24 Vanoaica, L. et al. Conditional deletion of ferritin h in mice reduces B and T lymphocyte populations. PLoS One 9, e89270, doi:10.1371/journal.pone.0089270 (2014).
25 Kerdiles, Y. M. et al. Foxol links homing and survival of naive T cells by regulating L-selectin, CCR7 and interleukin 7 receptor. Nat Immunol 10, 176-184, doi:10.1038/ni.1689 (2009).
26 Ouyang, W., Beckett, 0., Flavell, R. A. & Li, M. 0. An essential role of the Forkhead-box transcription factor Foxol in control of T cell homeostasis and tolerance. Immunity 30, 358-371, doi:10.1016/j.immuni.2009.02.003 (2009).
27 Chen, R. et al. In vivo RNA interference screens identify regulators of antiviral CD4(+) and CD8(+) T cell differentiation. Immunity 41, 325-338, doi:10.1016/j.immuni.2014.08.002 (2014).
28 Sanson, K. R. et al. Optimized libraries for CRISPR-Cas9 genetic screens with multiple modalities. Nat Commun 9, 5416, doi:10.1038/s41467-018-07901-8 (2018).
29 Fidler, I. J. Biological behavior of malignant melanoma cells correlated to their survival in vivo. Cancer Res 35, 218-224 (1975).
30 Overwijk, W. W. et al. Tumor regression and autoimmunity after reversal of a functionally tolerant state of self-reactive CD8+T cells. J Exp Med 198, 569-580, doi:10.1084/jem.20030590 (2003).
31 Churchman, M. L. et al. Synergism of FAK and tyrosine kinase inhibition in Ph(+) B-ALL. JCI Insight 1, doi:10.1172/jci.insight.86082 (2016).
32 Wei, J. et al. Autophagy enforces functional integrity of regulatory T cells by coupling environmental cues and metabolic homeostasis. Nat Immunol 17, 277-285, doi:10.1038/ni.3365 (2016).
33 Ivanova, N. B. et al. A stem cell molecular signature. Science 298, 601-604, doi:10.1126/science.1073823 (2002).
34 Ochsenbein, A. F. et al. CD27 expression promotes long-term survival of functional effector-memory CD8+cytotoxic T lymphocytes in HIV-infected patients. J Exp Med 200, 1407-1417, doi:10.1084/jem.20040717 (2004).
35 Harrington, L. E., Galvan, M., Baum, L. G., Altman, J. D. & Ahmed, R. Differentiating between memory and effector CD8 T cells by altered expression of cell surface 0-glycans. J Exp Med 191, 1241-1246 (2000).
36 Hurton, L. V. et al. Tethered IL-15 augments antitumor activity and promotes a stem-cell memory subset in tumor-specific T cells. Proc Natl Acad Sci USA 113, E7788-E7797, doi:10.1073/pnas.1610544113 (2016).
37 Yang, C. Y. et al. The transcriptional regulators Id2 and Id3 control the formation of distinct memory CD8+T cell subsets. Nat Immunol 12, 1221-1229, doi:10.1038/ni.2158 (2011).
38 Zhou, X. et al. Differentiation and persistence of memory CD8(+) T cells depend on T cell factor 1. Immunity 33, 229-240, doi:10.1016/j.immuni.2010.08.002 (2010).
39 Roychoudhuri, R. et al. BACH2 regulates CD8(+) T cell differentiation by controlling access of AP-1 factors to enhancers. Nat Immunol 17, 851-860, doi:10.1038/ni.3441 (2016).
40 Ichii, H., Sakamoto, A., Kuroda, Y. & Tokuhisa, T. Bc16 acts as an amplifier for the generation and proliferative capacity of central memory CD8+T cells. J Immunol 173, 883-891 (2004).
41 Wherry, E. J. et al. Molecular signature of CD8+T cell exhaustion during chronic viral infection. Immunity 27, 670-684, doi:10.1016/j.immuni.2007.09.006 (2007).
42 Waugh, K. A. et al. Molecular Profile of Tumor-Specific CD8+T Cell Hypofunction in a Transplantable Murine Cancer Model. J Immunol 197, 1477-1488, doi:10.4049/jimmuno1.1600589 (2016).
43 Man, K. et al. Transcription Factor IRF4 Promotes CD8(+) T Cell Exhaustion and Limits the Development of Memory-like T Cells during Chronic Infection. Immunity 47, 1129-1141 e1125, doi:10.1016/j.immuni.2017.11.021 (2017).
44 Wherry, E. J. & Kurachi, M. Molecular and cellular insights into T cell exhaustion. Nat Rev Immunol 15, 486-499, doi:10.1038/nri3862 (2015).
45 Sade-Feldman, M. et al. Defining T Cell States Associated with Response to Checkpoint Immunotherapy in Melanoma. Cell 175, 998-1013 e1020, doi:10.1016/j.ce11.2018.10.038 (2018).
46 Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213-1218, doi:10.1038/nmeth.2688 (2013).
47 Zeng, H. et al. mTORC1 couples immune signals and metabolic programming to establish T(reg)-cell function. Nature 499, 485-490, doi:10.1038/nature12297 (2013).
48 He, R. et al. Follicular CXCRS- expressing CD8(+) T cells curtail chronic viral infection. Nature 537, 412-428, doi:10.1038/nature19317 (2016).
49 Zhang, B. & Horvath, S. A general framework for weighted gene co-expression network analysis. Stat Appl Genet Mol Biol 4, Article17, doi:10.2202/1544-6115.1128 (2005).
50 Karmaus, P. W. F. et al. Metabolic heterogeneity underlies reciprocal fates of TH17 cell stemness and plasticity. Nature 565, 101-105, doi:10.1038/s41586-018-0806-7 (2019).
51 Li, H. & Durbin, R. Fast and accurate short read alignment with Burrows-Wheeler transform. Bioinformatics 25, 1754-1760, doi:10.1093/bioinformatics/btp324 (2009).
52 Li, H. et al. The Sequence Alignment/Map format and SAMtools. Bioinformatics 25, 2078-2079, doi:10.1093/bioinformatics/btp352 (2009).
53 Robinson, J. T. et al. Integrative genomics viewer. Nat Biotechnol 29, 24-26, doi:10.1038/nbt.1754 (2011).
54 Zhang, Y. et al. Model-based analysis of ChIP-Seq (MACS). Genome Biol 9, R137, doi:10.1186/gb-2008-9-9-r137 (2008).
55 Quinlan, A. R. & Hall, I. M. BEDTools: a flexible suite of utilities for comparing genomic features. Bioinformatics 26, 841-842, doi:10.1093/bioinformatics/btq033 (2010).
56 Law, C. W., Chen, Y., Shi, W. & Smyth, G. K. voom: Precision weights unlock linear model analysis tools for RNA-seq read counts. Genome Biol 15, R29, doi:10.1186/gb-2014-15-2-r29 (2014).
57 Bailey, T. L. et al. MEME SUITE: tools for motif discovery and searching. Nucleic Acids Res 37, W202-208, doi:10.1093/nar/gkp335 (2009).
58 Ramirez, F., Dundar, F., Diehl, S., Gruning, B. A. & Manke, T. deepTools: a flexible platform for exploring deep-sequencing data. Nucleic Acids Res 42, W187-191, doi:10.1093/nar/gku365 (2014).
59 Cuellar-Partida, G. et al. Epigenetic priors for identifying active transcription factor binding sites. Bioinformatics 28, 56-62, doi:10.1093/bioinformatics/btr614 (2012).
60 Krishnamoorthy, V. et al. The IRF4 Gene Regulatory Module Functions as a Read-Write Integrator to Dynamically Coordinate T Helper Cell Fate. Immunity 47, 481-497 e487, doi:10.1016/j.immuni.2017.09.001 (2017).
61 Sukumar, M. et al. Mitochondrial Membrane Potential Identifies Cells with Enhanced Stemness for Cellular Therapy. Cell Metab 23, 63-76, doi:10.1016/j.cmet.2015.11.002 (2016).
62 Moroz, A. et al. IL-21 enhances and sustains CD8+T cell responses to achieve durable tumor immunity: comparative evaluation of IL-2, IL-15, and IL-21. J Immunol 173, 900-909, doi:10.4049/jimmuno1.173.2.900 (2004).
63 Wang, W. et al. Effector T Cells Abrogate Stroma-Mediated Chemoresistance in Ovarian Cancer. Cell 165, 1092-1105, doi:10.1016/j.ce11.2016.04.009 (2016).
64 Sarkar, S. et al. Functional and genomic profiling of effector CD8 T cell subsets with distinct memory fates. J Exp Med 205, 625-640, doi:10.1084/jem.20071641 (2008).
65 Nakamura-Ishizu, A., Takizawa, H. & Suda, T. The analysis, roles and regulation of quiescence in hematopoietic stem cells. Development 141, 4656-4666, doi:10.1242/dev.106575 (2014).
66 Sprent, J. & Surh, C. D. Normal T cell homeostasis: the conversion of naive cells into memory-phenotype cells. Nat Immunol 12, 478-484 (2011).
67 Chang, J. T., Wherry, E. J. & Goldrath, A. W. Molecular regulation of effector and memory T cell differentiation. Nat Immunol 15, 1104-1115, doi:10.1038/ni.3031 (2014).
68 Macosko, E. Z. et al. Highly Parallel Genome-wide Expression Profiling of Individual Cells Using Nanoliter Droplets. Cell 161, 1202-1214, doi:10.1016/j.ce11.2015.05.002 (2015).
69 Khan, O. et al. TOX transcriptionally and epigenetically programs CD8(+) T cell exhaustion. Nature 571, 211-218, doi:10.1038/s41586-019-1325-x (2019).
70 Yao, C. et al. Single-cell RNA-seq reveals TOX as a key regulator of CD8(+) T cell persistence in chronic infection. Nat Immunol 20, 890-901, doi:10.1038/s41590-019-0403-4 (2019).
71 Alfei, F. et al. TOX reinforces the phenotype and longevity of exhausted T cells in chronic viral infection. Nature 571, 265-269, doi:10.1038/s41586-019-1326-9 (2019).
72 Scott, A. C. et al. TOX is a critical regulator of tumour-specific T cell differentiation. Nature 571, 270-274, doi:10.1038/s41586-019-1324-y (2019).
73 Miller, B. C. et al. Subsets of exhausted CD8(+) T cells differentially mediate tumor control and respond to checkpoint blockade. Nat Immunol 20, 326-336, doi:10.1038/s41590-019-0312-6 (2019).
74 Utzschneider, D. T. et al. T Cell Factor 1-Expressing Memory-like CD8(+) T Cells Sustain the Immune Response to Chronic Viral Infections. Immunity 45, 415-427, doi:10.1016/j.immuni.2016.07.021 (2016).
75 Ciofani, M. et al. A validated regulatory network for Th17 cell specification. Cell 151, 289-303, doi:10.1016/j.ce11.2012.09.016 (2012).
76 LaFleur, M. W. et al. A CRISPR-Cas9 delivery system for in vivo screening of genes in the immune system. Nat Commun 10, 1668, doi:10.1038/s41467-019-09656-2 (2019).
77 Doench, J. G. et al. Optimized sgRNA design to maximize activity and minimize off-target effects of CRISPR-Cas9. Nat Biotechnol 34, 184-191, doi:10.1038/nbt.3437 (2016).
78 Sinclair, L. V. et al. Control of amino-acid transport by antigen receptors coordinates the metabolic reprogramming essential for T cell differentiation. Nat Immunol 14, 500-508, doi:10.1038/ni.2556 (2013).
79 Atherly, L. O., Brehm, M. A., Welsh, R. M. & Berg, L. J. Tec kinases Itk and Rlk are required for CD8+T cell responses to virus infection independent of their role in CD4+T cell help. J Immunol 176, 1571-1581 (2006).
80 Blagih, J. et al. The energy sensor AMPK regulates T cell metabolic adaptation and effector responses in vivo. Immunity 42, 41-54, doi:10.1016/j.immuni.2014.12.030 (2015).
81 D′Souza, W. N., Chang, C. F., Fischer, A. M., Li, M. & Hedrick, S. M. The Erk2 MAPK regulates CD8 T cell proliferation and survival. J Immunol 181, 7617-7629 (2008).
82 Sullivan, B. M., Juedes, A., Szabo, S. J., von Herrath, M. & Glimcher, L. H. Antigen-driven effector CD8 T cell function regulated by T-bet. Proc Natl Acad Sci USA 100, 15818-15823, doi:10.1073/pnas.2636938100 (2003).
83 Geiger, R. et al. L-Arginine Modulates T Cell Metabolism and Enhances Survival and Anti-tumor Activity. Cell 167, 829-842 e813, doi:10.1016/j.ce11.2016.09.031 (2016).
84 Kawalekar, O. U. et al. Distinct Signaling of Coreceptors Regulates Specific Metabolism Pathways and Impacts Memory Development in CAR T Cells. Immunity 44, 380-390, doi:10.1016/j.immuni.2016.01.021 (2016).
85 Quigley, M. et al. Transcriptional analysis of HIV-specific CD8+ T cells shows that PD-1 inhibits T cell function by upregulating BATF. Nat Med 16, 1147-1151, doi:10.1038/nm.2232 (2010).
86 Xin, G. et al. A Critical Role of IL-21-Induced BATF in Sustaining CD8-T-Cell-Mediated Chronic Viral Control. Cell Rep 13, 1118-1124, doi:10.1016/j.celrep.2015.09.069 (2015).
87 Shifrut, E. et al. Genome-wide CRISPR Screens in Primary Human T Cells Reveal Key Regulators of Immune Function. Cell 175, 1958-1971 e1915, doi:10.1016/j.ce11.2018.10.024 (2018).
88 Manguso, R. T. et al. In vivo CRISPR screening identifies Ptpn2 as a cancer immunotherapy target. Nature 547, 413-418, doi:10.1038/nature23270 (2017).

LIST OF SEQUENCES
sgRegnase-1 #1 nucleic acid sequence
SEQ ID NO: 1
AAGGCAGTGGTTTCTTACGA
sgRegnase-1 #2 nucleic acid sequence
SEQ ID NO: 2
GGAGTGGAAACGCTTCATCG
sgBatf #1 nucleic acid sequence
SEQ ID NO: 3
AGAGATCAAACAGCTCACCG
sgBatf #2 nucleic acid sequence
SEQ ID NO: 4
AGGACTCATCTGATGATGTG
sgPtpn2 #1 nucleic acid sequence
SEQ ID NO: 5
AAGAAGTTACATCTTAACAC
sgPtpn2 #2 nucleic acid sequence
SEQ ID NO: 6
CACTCTATGAGGATAGTCAT
sgSocs1 #1 nucleic acid sequence
SEQ ID NO: 7
TGATGCGCCGGTAATCGGAG
sgSocs1 #2 nucleic acid sequence
SEQ ID NO: 8
TGGTGCGCGACAGTCGCCAA
sgAgps nucleic acid sequence
SEQ ID NO: 9
GTACCAATGAGTGCAAAGCG
non-targeting control sgRNA nucleic acid sequence
SEQ ID NO: 10
ATGACACTTACGGTACTCGT
Nextera NGS-F nucleic acid sequence
SEQ ID NO: 11
TCGTCGGCAGCGTCAGATGTGTATAAGAGACAGttgtggaaaggacgaaa
caccg
Nextera NGS-R nucleic acid sequence
SEQ ID NO: 12
GTCTCGTGGGCTCGGAGATGTGTATAAGAGACAGccactttttcaagttg
ataacgg
Bcl211-F nucleic acid sequence
SEQ ID NO: 13
GACAAGGAGATGCAGGTATTGG
Bcl211-R nucleic acid sequence
SEQ ID NO: 14
TCCCGTAGAGATCCACAAAAGT
Ifng-F nucleic acid sequence
SEQ ID NO: 15
ACAGCAAGGCGAAAAAGGATG
Ifng-R nucleic acid sequence
SEQ ID NO: 16
TGGTGGACCACTCGGATGA
Irf4-F nucleic acid sequence
SEQ ID NO: 17
TCCGACAGTGGTTGATCGAC
Irf4-R nucleic acid sequence
SEQ ID NO: 18
CCTCACGATTGTAGTCCTGCTT
Gzma-F nucleic acid sequence
SEQ ID NO: 19
TGCTGCCCACTGTAACGTG
Gzma-R nucleic acid sequence
SEQ ID NO: 20
GGTAGGTGAAGGATAGCCACAT
Gzmb-F nucleic acid sequence
SEQ ID NO: 21
CCACTCTCGACCCTACATGG
Gzmb-R nucleic acid sequence
SEQ ID NO: 22
GGCCCCCAAAGTGACATTTATT
Actin-F nucleic acid sequence
SEQ ID NO: 23
CGCCACCAGTTCGCCATGGA
Actin-R nucleic acid sequence
SEQ ID NO: 24
TACAGCCCGGGGAGCATCGT
human BATF amino acid sequence
SEQ ID NO: 25
MPHSSDSSDSSFSRSPPPGKQDSSDDVRRVQRREKNRIAAQKSRQRQTQK
ADTLHLESEDLEKQNAALRKEIKQLTEELKYFTSVLNSHEPLCSVLAAST
PSPPEVVYSAHAFHQPHVSSPRFQP
mouse BATF amino acid sequence
SEQ ID NO: 26
MPHSSDSSDSSFSRSPPPGKQDSSDDVRKVQRREKNRIAAQKSRQRQTQK
ADTLHLESEDLEKQNAALRKEIKQLTEELKYFTSVLSSHEPLCSVLASGT
PSPPEVVYSAHAFHQPHISSPRFQP
human BATF nucleotide sequence
SEQ ID NO: 27
AAAGCGAGCGACATGTCCCTTTGGGGAGCAGTCCCTCTGCACCCCAGAGT
GAGGAGGACGCAGGGGTCAGAGGTGGCTACAGGGCAGGCAGAGGAGGCAC
CTGTAGGGGGTGGTGGGCTGGTGGCCCAGGAGAAGTCAGGAAGGGAGCCC
AGCTGGTGACAAGAGAGCCCAGAGGTGCCTGGGGCTGAGTGTGAGAGCCC
GGAAGATTTCAGCCATGCCTCACAGCTCCGACAGCAGTGACTCCAGCTTC
AGCCGCTCTCCTCCCCCTGGCAAACAGGACTCATCTGATGATGTGAGAAG
AGTTCAGAGGAGGGAGAAAAATCGTATTGCCGCCCAGAAGAGCCGACAGA
GGCAGACACAGAAGGCCGACACCCTGCACCTGGAGAGCGAAGACCTGGAG
AAACAGAACGCGGCTCTACGCAAGGAGATCAAGCAGCTCACAGAGGAACT
GAAGTACTTCACGTCGGTGCTGAACAGCCACGAGCCCCTGTGCTCGGTGC
TGGCCGCCAGCACGCCCTCGCCCCCCGAGGTGGTGTACAGCGCCCACGCA
TTCCACCAACCTCATGTCAGCTCCCCGCGCTTCCAGCCCTGAGCTTCCGA
TGCGGGGAGAGCAGAGCCTCGGGAGGGGCACACAGACTGTGGCAGAGCTG
CGCCCATCCCGCAGAGGCCCCTGTCCACCTGGAGACCCGGAGACAGAGGC
CTGGACAAGGAGTGAACACGGGAACTGTCACGACTGGAAGGGCGTGAGGC
CTCCCAGCAGTGCCGCAGCGTTTCGAGGGGCGTGTGCTGGACCCCACCAC
TGTGGGTTGCAGGCCCAATGCAGAAGAGTATTAAGAAAGATGCTCAAGTC
CCATGGCACAGAGCAAGGCGGGCAGGGAACGGTTATTTTTCTAAATAAAT
GCTTTAAAAGAAA
mouse BATF nucleotide sequence
SEQ ID NO: 28
GCAGTCCCTCTGCACCCGAGAGAGAGGAGGACGCAGGGGTCTGTCAGAGG
TTGCTGTTGGGCAAGCAGGGGAGGTACCTGTGGAAGGTGGTGTGCTGGTG
GCCCCCTAGCAGTCAAGAAGGGGAGCCAGCTAGTGAGAAGATCGCCCAGA
GGCATCTGGGACGGTGTGGGAGAGCCCGGAAGATTAGAACCATGCCTCAC
AGCTCCGACAGCAGTGACTCCAGCTTCAGCCGCTCTCCTCCCCCTGGCAA
ACAGGACTCATCTGATGATGTGAGGAAAGTTCAGAGGAGAGAGAAGAATC
GCATCGCTGCCCAGAAGAGCCGACAGAGACAGACACAGAAAGCCGACACC
CTTCACCTGGAGAGTGAGGACCTGGAGAAACAGAACGCAGCTCTCCGCAA
AGAGATCAAACAGCTCACCGAGGAGCTCAAGTACTTCACATCAGTGCTGA
GCAGCCACGAGCCCCTGTGCTCCGTGCTGGCCAGTGGCACCCCCTCGCCC
CCCGAGGTGGTATACAGTGCCCATGCCTTCCACCAGCCTCACATCAGCTC
GCCACGCTTCCAGCCCTGACCTTCTGGACAAGAAGGGCGATGCTACTCCC
GTGATCCCTTGGAGGGGCATGTAAACTGAGGCCGGGCTGCCCTCATACCT
CTACCCAGAGGCCCAGTGGCAGAGGCCTGGACAAGTATTGAACACAAGAA
CTGTAGTGGTCAGAGGGACTTAAGGCCTCCCAGGGAAGTATAGTCAATGT
ACTGGACTCTCCCAGGGAAGTCGAGCCAATGTACTGGACCCAAAAAATGA
CAAGTCAACCCTGGACTGTCATGAATGATGCCCAAAATACACAGCACAGA
GGGAGGAGGGCAGGGGGTGGATAGTTTTCTAAATAAATATTTTCTAAAAA
ACCA
gRNA-1, N is A, T, C, or G
SEQ ID NO: 29
TTCACACCATCACGACGCGTNGG
gRNA-2, N is A, T, C, or G
SEQ ID NO: 30
TGGGGGCAGCTTGGCCGCTCNGG
g-RNA-3, N is A, T, C, or G
SEQ ID NO: 31
TATGCCCCCTGATGACCCACNGG
gRNA-4, N is A, T, C, or G
SEQ ID NO: 32
AAGGAGGTCTTCTCCTGCCGNGG
gRNA-5, N is A, T, C, or G
SEQ ID NO: 33
GTGATGGGCACGTCGGGCCGNGG
g-RNA-6, N is A, T, C, or G
SEQ ID NO: 34
CAGCTCCCTCTAGTCCCGCGNGG
control gRNA, N is A, T, C, or G
SEQ ID NO: 35
GCUUGUGGAUGUUGCGGAAGNGG
gRNA-1
SEQ ID NO: 36
TTCACACCATCACGACGCGT
gRNA-2
SEQ ID NO: 37
TGGGGGCAGCTTGGCCGCTC
g-RNA-3
SEQ ID NO: 38
TATGCCCCCTGATGACCCAC
gRNA-4
SEQ ID NO: 39
AAGGAGGTCTTCTCCTGCCG
gRNA-5
SEQ ID NO: 40
GTGATGGGCACGTCGGGCCG
g-RNA-6
SEQ ID NO: 41
CAGCTCCCTCTAGTCCCGCG
sgRc3h1 nucleic acid sequence
SEQ ID NO: 42
GGTAGAGGGTTACTACCCGG

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of enhancing expansion and/or persistence and/or an anti-tumor function and/or an anti-infection function of a T cell, comprising modifying one or more genes selected from Regnase-1 (REGNASE-1, Zc3h12a, MCPIP1), Ptpn2, Socs1, Agps, Rc3h1, and Rcor1 or gene product(s) thereof in the T cell such that the expression and/or function of said gene(s) or gene product(s) in the T cell is reduced or eliminated.

2-26. (canceled)

27. A modified T cell produced by the method of claim 1.

27-32. (canceled)

33. A pharmaceutical composition comprising the modified T cell of claim 27 and a pharmaceutically acceptable carrier and/or excipient.

34. A method of treating a disease in a subject in need thereof comprising administering to the subject an effective amount of the modified T cells of claim 27 or a pharmaceutical composition comprising said modified T cells and a pharmaceutically acceptable carrier and/or excipient.

35-43. (canceled)

44. A method of enhancing expansion and/or persistence and/or an anti-tumor function and/or an anti-infection function of a T cell, comprising increasing the expression of Batf gene and/or enhancing the function of BATF protein in the T cell.

45. The method of claim 44, wherein the T cell is selected from a CD8⁺αβ T cell receptor (TCR) T cell, a CD4⁺ αβ TCR T cell, a regulatory T cell, a natural killer T (NKT) cell, and a γϵ T cell.

46-47. (canceled)

48. The method of claim 44, wherein the T cell is further engineered to express a T cell receptor or a chimeric antigen receptor (CAR).

49. The method of claim 48, wherein the CAR targets a tumor antigen or an infectious antigen.

50. The method of claim 44, wherein the method comprises introducing into the T cell a polynucleotide encoding a BATF protein, or functional fragment or derivative thereof.

51. The method of claim 50, wherein the polynucleotide encoding a BATF protein comprises the nucleotide sequence of SEQ ID NO: 27, or a nucleotide sequence having at least 80% identity thereof, or wherein the BATF protein encoded by the polynucleotide comprises the amino acid sequence of SEQ ID NO: 25, or an amino acid sequence having at least 80% identity thereof.

52. (canceled)

53. The method of claim 50, wherein the polynucleotide encoding a BATF protein, or functional fragment or derivative thereof, is introduced into the T cell in a recombinant vector.

54. The method of claim 53, wherein the recombinant vector is a viral vector, or a non-viralRNA and/or DNA vector.

55. The method of claim 54, wherein the viral vector is a retroviral vector, a lentiviral vector, an adenoviral vector, an adeno-associated virus vector, an alphaviral vector, a herpes virus vector, or a vaccinia virus vector.

56-57. (canceled)

58. The method of , further comprising claim 44, further comprising modifying one or more additional genes or gene products in the T cell such that the expression and/or function of said additional gene(s) or gene product(s) in said T cell is reduced or eliminated, wherein said additional gene(s) or gene product(s) are selected from Regnase-1 (REGNASE-1, Zc3h12a, MCPIP 1), Ptpn2, Socs 1 , Agps, Rc3h1, and Rcor 1 .

59. (canceled)

60. The method of claim 58, wherein the modifying of one or more additional genes comprises disrupting said gene(s) with a site-specific nuclease, or administering an RNA interference (RNAi) molecule or an antisense oligonuclcotide, or modifying of one or more additional gene products comprising adminstering one or more of a small molecule inhibitor, a peptide, an antibody or antibody fragment, and an aptamer.

61. The method of claim 60, wherein the site-specific nuclease comprises a Cas protein and a guide RNA, or a zinc finger nuclease (ZFN), or a TALEN nuclease, or a mega-TALEN nuclease.

62. The method of claim 61, wherein the Cas protein is a Cas9 protein.

63. (canceled)

64. The method of claim 61, wherein the guide RNA comprises TTCACACCATCACGACGCGTNGG (SEQ ID NO: 29), CAGCTCCCTCTAGTCCCGCGNGG (SEQ ID NO: 34), TTCACACCATCACGACGCGT (SEQ ID NO: 36), or CAGCTCCCTCTAGTCCCGCG (SEQ ID NO: 41), or a nucleotide sequence having at least 80% identity thereof.

65-77. (canceled)

78. A modified T cell produced by the method of claim 44.

79-83. (canceled)

84. A pharmaceutical composition comprising the modified T cell of claim 78 and a pharmaceutically acceptable carrier and/or excipient.

85. A method of treating a disease in a subject in need thereof comprising administering to the subject an effective amount of the modified T cells of claim 78 or a pharmaceutical composition comprising said modified T cells and a pharmaceutically acceptable carrier and/or excipient.

86-94. (canceled)

95. A method of improving mitochondrial biogenesis and/or function in a T cell comprising modifying one of more genes selected from Regnase-1 (REGNASE-1, Zc3h12a, MCPIP1), Ptpn2, Socs1, Agps, Rc3h1, and Rcor1 or gene product(s) thereof in the T cell such that the expression and/or function of said gene(s) or gene product(s) in the T cell is reduced or eliminated and/or increasing the expression of Batf gene and/or enhancing the function of BATF protein in the T cell.

96. (canceled)

97. An isolated polynucleotide, comprising the nucleotide sequence of any one of SEQ ID NOs: 1-9, 29-34 and 36-42, or a nucleotide sequence having at least 80% identity thereof.

98-140. (canceled)
